SEQUENCE LISTING (124 sequences)

SEQ ID NO: 1 - protein, 113 aa, synthetic construct
QVQLQQPGTELVKPGASVKLSCKASGYTFTSYWLQWVKQRPGQGLEWIGEIDPSDSYTNYNQKFKGKATLTVDTSSSTAYMQLSSLTSEDSAVYYCATLTGSWGQGTTLAVSS

SEQ ID NO: 2 - protein, 112 aa, synthetic construct
DVLLTQTPLSLPVSLGDQASISCRASQSIVDSNGNTFLEWYLQKSGQSPKVLIYKVSNRFSGVPDRFSGRGSGTDFTLKISRVEAEDLGVYYCFQGSHVPYTFGGGTKLEIK

SEQ ID NO: 3 - protein, 257 aa, synthetic construct
MQVQLQQPGTELVKPGASVKLSCKASGYTFTSYWLQWVKQRPGQGLEWIGEIDPSDSYTNYNQKFKGKATLTVDTSSSTAYMQLSSLTSEDSAVYYCATLTGSWGQGTTLAVSSGGGGSGGGGSGGGGSGGGGSDVLLTQTPLSLPVSLGDQASISCRASQSIVDSNGNTFLEWYLQKSGQSPKVLIYKVSNRFSGVPDRFSGRGSGTDFTLKISRVEAEDLGVYYCFQGSHVPYTFGGGTKLEIKLPYTGHHHHHH

SEQ ID NO: 4 - protein, 278 aa, synthetic construct
MDYKDHDGDYKDHDIDYKDDDDKQVQLVQSGAEVKKPGASVKVSCKASGYTFTSYWMHWVRQAPGQGLEWMGEIDPSDSYTSYAQKFQGRVTMTRDTSTSTVYMELSSLRSEDTAVYYCATLTGSWGQGTTLAVSSGGGGSGGGGSGGGGSDVVMTQSPLSLPVTLGQPASISCRASQSIVDSNGNTFLEWFQQRPGQSPRRLIYKVSNRDSGVPDRFSGSGSGTDFTLKISRVEAEDVGVYYCFQGSHVPYTFGGGTKLEIKYPYDVPDYAHHHHHH

SEQ ID NO: 5 - protein, 468 aa, synthetic construct
MDYKDHDGDYKDHDIDYKDDDDKQVQLQQPGTELVKPGASVKLSCKASGYTFTSYWLQWVKQRPGQGLEWIGEIDPSDSYTNYNQKFKGKATLTVDTSSSTAYMQLSSLTSEDSAVYYCATLTGSWGQGTTLAVSSGGGGSGGGGSGGGGSDVLLTQTPLSLPVSLGDQASISCRASQSIVDSNGNTFLEWYLQKSGQSPKVLIYKVSNRFSGVPDRFSGRGSGTDFTLKISRVEAEDLGVYYCFQGSHVPYTFGGGTKLEIKYPYDVPDYAGGGGSGGGGSGGGGSGGGGSRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 6 - protein, 8 aa, synthetic construct
GYTFTSYW

SEQ ID NO: 7 - protein, 8 aa, synthetic construct
IDPSDSYT

SEQ ID NO: 8 - skipped

SEQ ID NO: 9 - protein, 11 aa, synthetic construct
QSIVDSNGNTF

SEQ ID NO: 10 - skipped

SEQ ID NO: 11 - protein, 7 aa, synthetic construct
FQGSHVP

SEQ ID NO: 12 - protein, 113 aa, synthetic construct
QVQLQQPGAELVKPGASVKLSCKASDYTFTNYWIQWVKQRPGQGLEWIGEIDPSDNYTNCNPEFKDKATLTVDTSSSTAYMHLNSLTSEDSAVYYCGNTKGWWGQGTLVTVSA

SEQ ID NO: 13 - protein, 112 aa, synthetic construct
DVLMTQTPLSLPVSLGDQASISCRSSQNIVNSNGDTYLEWYLQKPGQSPKLLIYKVSNRFSGVPDRFSGSGSGTDFTLKISGAEAEDLGVYYCFQGSHVPLTFGAGTKLELK

SEQ ID NO: 14 - protein, 8 aa, synthetic construct
DYTFTNYW

SEQ ID NO: 15 - protein, 8 aa, synthetic construct
IDPSDNYT

SEQ ID NO: 16 - protein, 248 aa, synthetic construct
MQVQLQQPGAELVKPGASVKLSCKASDYTFTNYWIQWVKQRPGQGLEWIGEIDPSDNYTNCNPEFKDKATLTVDTSSSTAYMHLNSLTSEDSAVYYCGNTKGWWGQGTLVTVSAGGGGSGGGGSGGGGSDVLMTQTPLSLPVSLGDQASISCRSSQNIVNSNGDTYLEWYLQKPGQSPKLLIYKVSNRFSGVPDRFSGSGSGTDFTLKISGAEAEDLGVYYCFQGSHVPLTFGAGTKLELKGHHHHHH

SEQ ID NO: 17 - protein, 11 aa, synthetic construct
QNIVNSNGDTY

SEQ ID NO: 18 - skipped

SEQ ID NO: 19 - protein, 7 aa, synthetic construct
FQGSHVP

SEQ ID NO: 20 - protein, 441 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 21 - protein, 15 aa, synthetic construct
MAEPRQEFEVMEDHA

SEQ ID NO: 22 - protein, 15 aa, synthetic construct
RQEFEVMEDHAGTYG

SEQ ID NO: 23 - protein, 15 aa, synthetic construct
EVMEDHAGTYGLGDR

SEQ ID NO: 24 - protein, 15 aa, synthetic construct
DHAGTYGLGDRKDQG

SEQ ID NO: 25 - protein, 15 aa, synthetic construct
TYGLGDRKDQGGYTM

SEQ ID NO: 26 - protein, 15 aa, synthetic construct
GDRKDQGGYTMHQDQ

SEQ ID NO: 27 - protein, 15 aa, synthetic construct
DQGGYTMHQDQEGDT

SEQ ID NO: 28 - protein, 15 aa, synthetic construct
YTMHQDQEGDTDAGL

SEQ ID NO: 29 - protein, 15 aa, synthetic construct
QDQEGDTDAGLKESP

SEQ ID NO: 30 - protein, 15 aa, synthetic construct
GDTDAGLKESPLQTP

SEQ ID NO: 31 - protein, 15 aa, synthetic construct
AGLKESPLQTPTEDG

SEQ ID NO: 32 - protein, 15 aa, synthetic construct
ESPLQTPTEDGSEEP

SEQ ID NO: 33 - protein, 15 aa, synthetic construct
QTPTEDGSEEPGSET

SEQ ID NO: 34 - protein, 15 aa, synthetic construct
EDGSEEPGSETSDAK

SEQ ID NO: 35 - protein, 15 aa, synthetic construct
EEPGSETSDAKSTPT

SEQ ID NO: 36 - protein, 15 aa, synthetic construct
SETSDAKSTPTAEAE

SEQ ID NO: 37 - protein, 15 aa, synthetic construct
DAKSTPTAEAEEAGI

SEQ ID NO: 38 - protein, 15 aa, synthetic construct
TPTAEAEEAGIGDTP

SEQ ID NO: 39 - protein, 15 aa, synthetic construct
EAEEAGIGDTPSLED

SEQ ID NO: 40 - protein, 15 aa, synthetic construct
AGIGDTPSLEDEAAG

SEQ ID NO: 41 - protein, 15 aa, synthetic construct
DTPSLEDEAAGHVTQ

SEQ ID NO: 42 - protein, 15 aa, synthetic construct
LEDEAAGHVTQARMV

SEQ ID NO: 43 - protein, 15 aa, synthetic construct
AAGHVTQARMVSKSK

SEQ ID NO: 44 - protein, 15 aa, synthetic construct
VTQARMVSKSKDGTG

SEQ ID NO: 45 - protein, 15 aa, synthetic construct
RMVSKSKDGTGSDDK

SEQ ID NO: 46 - protein, 15 aa, synthetic construct
KSKDGTGSDDKKAKG

SEQ ID NO: 47 - protein, 15 aa, synthetic construct
GTGSDDKKAKGADGK

SEQ ID NO: 48 - protein, 15 aa, synthetic construct
DDKKAKGADGKTKIA

SEQ ID NO: 49 - protein, 15 aa, synthetic construct
AKGADGKTKIATPRG

SEQ ID NO: 50 - protein, 15 aa, synthetic construct
DGKTKIATPRGAAPP

SEQ ID NO: 51 - protein, 15 aa, synthetic construct
KIATPRGAAPPGQKG

SEQ ID NO: 52 - protein, 15 aa, synthetic construct
PRGAAPPGQKGQANA

SEQ ID NO: 53 - protein, 15 aa, synthetic construct
APPGQKGQANATRIP

SEQ ID NO: 54 - protein, 15 aa, synthetic construct
QKGQANATRIPAKTP

SEQ ID NO: 55 - protein, 15 aa, synthetic construct
ANATRIPAKTPPAPK

SEQ ID NO: 56 - protein, 15 aa, synthetic construct
RIPAKTPPAPKTPPS

SEQ ID NO: 57 - protein, 15 aa, synthetic construct
KTPPAPKTPPSSGEP

SEQ ID NO: 58 - protein, 15 aa, synthetic construct
APKTPPSSGEPPKSG

SEQ ID NO: 59 - protein, 15 aa, synthetic construct
PPSSGEPPKSGDRSG

SEQ ID NO: 60 - protein, 15 aa, synthetic construct
GEPPKSGDRSGYSSP

SEQ ID NO: 61 - protein, 15 aa, synthetic construct
KSGDRSGYSSPGSPG

SEQ ID NO: 62 - protein, 15 aa, synthetic construct
RSGYSSPGSPGTPGS

SEQ ID NO: 63 - protein, 15 aa, synthetic construct
SSPGSPGTPGSRSRT

SEQ ID NO: 64 - protein, 15 aa, synthetic construct
SPGTPGSRSRTPSLP

SEQ ID NO: 65 - protein, 15 aa, synthetic construct
PGSRSRTPSLPTPPT

SEQ ID NO: 66 - protein, 15 aa, synthetic construct
SRTPSLPTPPTREPK

SEQ ID NO: 67 - protein, 15 aa, synthetic construct
SLPTPPTREPKKVAV

SEQ ID NO: 68 - protein, 15 aa, synthetic construct
PPTREPKKVAVVRTP

SEQ ID NO: 69 - protein, 15 aa, synthetic construct
EPKKVAVVRTPPKSP

SEQ ID NO: 70 - protein, 15 aa, synthetic construct
VAVVRTPPKSPSSAK

SEQ ID NO: 71 - protein, 15 aa, synthetic construct
RTPPKSPSSAKSRLQ

SEQ ID NO: 72 - protein, 15 aa, synthetic construct
KSPSSAKSRLQTAPV

SEQ ID NO: 73 - protein, 15 aa, synthetic construct
SAKSRLQTAPVPMPD

SEQ ID NO: 74 - protein, 15 aa, synthetic construct
RLQTAPVPMPDLKNV

SEQ ID NO: 75 - protein, 15 aa, synthetic construct
APVPMPDLKNVKSKI

SEQ ID NO: 76 - protein, 15 aa, synthetic construct
MPDLKNVKSKIGSTE

SEQ ID NO: 77 - protein, 15 aa, synthetic construct
KNVKSKIGSTENLKH

SEQ ID NO: 78 - protein, 15 aa, synthetic construct
SKIGSTENLKHQPGG

SEQ ID NO: 79 - protein, 15 aa, synthetic construct
STENLKHQPGGGKVQ

SEQ ID NO: 80 - protein, 15 aa, synthetic construct
LKHQPGGGKVQIINK

SEQ ID NO: 81 - protein, 15 aa, synthetic construct
PGGGKVQIINKKLDL

SEQ ID NO: 82 - protein, 15 aa, synthetic construct
KVQIINKKLDLSNVQ

SEQ ID NO: 83 - protein, 15 aa, synthetic construct
INKKLDLSNVQSKCG

SEQ ID NO: 84 - protein, 15 aa, synthetic construct
LDLSNVQSKCGSKDN

SEQ ID NO: 85 - protein, 15 aa, synthetic construct
NVQSKCGSKDNIKHV

SEQ ID NO: 86 - protein, 15 aa, synthetic construct
KCGSKDNIKHVPGGG

SEQ ID NO: 87 - protein, 15 aa, synthetic construct
KDNIKHVPGGGSVQI

SEQ ID NO: 88 - protein, 15 aa, synthetic construct
KHVPGGGSVQIVYKP

SEQ ID NO: 89 - protein, 15 aa, synthetic construct
GGGSVQIVYKPVDLS

SEQ ID NO: 90 - protein, 15 aa, synthetic construct
VQIVYKPVDLSKVTS

SEQ ID NO: 91 - protein, 15 aa, synthetic construct
YKPVDLSKVTSKCGS

SEQ ID NO: 92 - protein, 15 aa, synthetic construct
DLSKVTSKCGSLGNI

SEQ ID NO: 93 - protein, 15 aa, synthetic construct
VTSKCGSLGNIHHKP

SEQ ID NO: 94 - protein, 15 aa, synthetic construct
CGSLGNIHHKPGGGQ

SEQ ID NO: 95 - protein, 15 aa, synthetic construct
GNIHHKPGGGQVEVK

SEQ ID NO: 96 - protein, 15 aa, synthetic construct
HKPGGGQVEVKSEKL

SEQ ID NO: 97 - protein, 15 aa, synthetic construct
GGQVEVKSEKLDFKD

SEQ ID NO: 98 - protein, 15 aa, synthetic construct
EVKSEKLDFKDRVQS

SEQ ID NO: 99 - protein, 15 aa, synthetic construct
EKLDFKDRVQSKIGS

SEQ ID NO: 100 - protein, 15 aa, synthetic construct
FKDRVQSKIGSLDNI

SEQ ID NO: 101 - protein, 15 aa, unidentified
VQSKIGSLDNITHVP

SEQ ID NO: 102 - protein, 15 aa, synthetic construct
IGSLDNITHVPGGGN

SEQ ID NO: 103 - protein, 15 aa, synthetic construct
DNITHVPGGGNKKIE

SEQ ID NO: 104 - protein, 15 aa, synthetic construct
HVPGGGNKKIETHKL

SEQ ID NO: 105 - protein, 15 aa, synthetic construct
GGNKKIETHKLTFRE

SEQ ID NO: 106 - protein, 15 aa, synthetic construct
KIETHKLTFRENAKA

SEQ ID NO: 107 - protein, 15 aa, synthetic construct
HKLTFRENAKAKTDH

SEQ ID NO: 108 - protein, 15 aa, synthetic construct
FRENAKAKTDHGAEI

SEQ ID NO: 109 - protein, 15 aa, synthetic construct
AKAKTDHGAEIVYKS

SEQ ID NO: 110 - protein, 15 aa, synthetic construct
TDHGAEIVYKSPVVS

SEQ ID NO: 111 - protein, 15 aa, synthetic construct
AEIVYKSPVVSGDTS

SEQ ID NO: 112 - protein, 15 aa, synthetic construct
YKSPVVSGDTSPRHL

SEQ ID NO: 113 - protein, 15 aa, synthetic construct
VVSGDTSPRHLSNVS

SEQ ID NO: 114 - protein, 15 aa, synthetic construct
DTSPRHLSNVSSTGS

SEQ ID NO: 115 - protein, 15 aa, synthetic construct
RHLSNVSSTGSIDMV

SEQ ID NO: 116 - protein, 15 aa, synthetic construct
NVSSTGSIDMVDSPQ

SEQ ID NO: 117 - protein, 15 aa, synthetic construct
TGSIDMVDSPQLATL

SEQ ID NO: 118 - protein, 15 aa, synthetic construct
DMVDSPQLATLADEV

SEQ ID NO: 119 - protein, 15 aa, synthetic construct
SPQLATLADEVSASL

SEQ ID NO: 120 - protein, 15 aa, synthetic construct
ATLADEVSASLAKQG

SEQ ID NO: 121 - protein, 15 aa, synthetic construct
TLADEVSASLAKQGL

SEQ ID NO: 122 - protein, 11 aa, synthetic construct
ANATRIPAKTP

SEQ ID NO: 123 - protein, 4 aa, synthetic construct
VAVV

SEQ ID NO: 124 - protein, 4 aa, synthetic construct
KVAV

(19) United States
(12) Patent Application Publication
(10) Pub. No.: US 2024/0352102 A1
(43) Pub. Date: Oct. 24, 2024

(54) COMPOSITIONS FOR TREATING TAUOPATHIES AND METHODS OF USE THEREOF

(71) Applicants: The United States Government As Represented By The Department Of Veterans Affairs, Washington, DC (US); University of Washington, Seattle, WA (US)

(72) Inventors: BRIAN C. KRAEMER, Seattle, WA (US); PAMELA MCMILLAN, Seattle, WA (US)

(21) Appl. No.: 18/424,638

(22) Filed: Jan. 26, 2024

Related U.S. Application Data
(60) Provisional application No. 63/481,965, filed on Jan. 27, 2023.

(51) Int. Cl. C07K 16/18; A61K 39/00

(52) U.S. Cl. CPC C07K 16/18; A61K 2039/505; C07K 2317/24; C07K 2317/55; C07K 2317/565

(57) ABSTRACT

Disclosed herein are antibodies that bind to aggregated tau/RNA complexes. Also disclosed herein are methods for treating a taupathy, dementia, ocular pharyngeal muscular dystrophy, or inhibiting microtubule polymerization with antibodies that bind to aggregated tau/RNA complexes.

### CROSS REFERENCE TO RELATED APPLICATIONS

This application claims the benefit of the filing date of U.S. Provisional Application No. 63/481,965, filed on Jan. 27, 2023. The content of this earlier filed application is hereby incorporated by reference in its entirety.

### STATEMENT REGARDING FEDERALLY FUNDED RESEARCH

This invention was made with government support under grant numbers and BX002619 and BX004044 awarded by United States Department of Veterans Affairs, and grant numbers AG055474 and AG066567 awarded by the National Institutes of Health. The government has certain rights in the invention.

### REFERENCE TO A SEQUENCE LISTING

The present application contains a Sequence Listing that is submitted concurrent with the filing of this application in XML format, containing the file name “37759_0519P1_SL.xml,” created on Jan. 26, 2024, and having a size of 110,592 bytes. The Sequence Listing is hereby incorporated by reference pursuant into the present application in its entirety.

### BACKGROUND

The brain protein tau, a natively unstructured protein encoded by the MAPT gene, performs an important physiological role in neurons by binding to and modulating neuronal microtubule stability (Brunden, K. R. et al., Nature Reviews Drug Discovery 2009, 8 (10), 783-793; Baas, P. W. et al., Trends Cell Biol 2019, 29 (6), 452-461; Gustke, N. et al., Biochemistry 1994, 33 (32), 9511-9522; and Binder, L. I., et al., J Cell Biol 1985, 101 (4), 1371-8). This activity helps to support the extensive processes neurons extend to conduct neuronal chemical and electrical signaling through axons (Ittner, A. et al., Neuron 2018, 99 (1), 13-27; and Frere, S., et al. Neuron 2018, 97 (1), 32-58). Under neuronal stress or in disease states, tau is often hyper-phosphorylated or altered by other post-translational modifications (PTMs) resulting in a propensity to self-associate and produce detergent insoluble protein aggregates including paired helical filaments and neurofibrillary tangles (NFTs) (Fontaine, S. N. et al., Cell Mol Life Sci 2015, 72 (10), 1863-79; and Sabbagh, J. J. et al., Frontiers in Neuroscience 2016, 10 (3)). Neurons exhibit complex patterns of tau expression with multiple splice isoforms and a myriad of PTMs controlling tau function (Goedert, M. et al., Neuron 1989, 3 (4), 519-526; Wang, J.-Z., et al. Nature Medicine 1996, 2 (8), 871-875; and Wang, Y. et al., Nature Reviews Neuroscience 2016, 17 (1), 22-35). Tau deposits may take many pathological forms depending on the associated disorder. Tauopathies, or disorders with primary insoluble tau deposits as hallmarks, include Alzheimer's disease, Pick disease, progressive supranuclear palsy, corticobasal degeneration, chronic traumatic encephalopathy, and globular glial tauopathy (Strang, K. H. et al., Laboratory Investigation 2019, 99 (7), 912-928; and Iqbal, K., et al. Tau and neurodegenerative disease: the story so far. Nature Reviews Neurology 2016, 12 (1), 15-27). There are no disease-modifying therapeutics for ameliorating pathological tau; new mechanistic targets and therapeutic strategies for these disorders are needed.

### SUMMARY OF THE INVENTION

Disclosed herein are antibodies and fragments thereof that can bind to aggregated tau/RNA complexes. In some aspects, the antibodies and fragments thereof can comprise sequences disclosed in Table 2.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are isolated antibodies comprising a light chain variable region amino acid sequence of SEQ ID NO: 2 and a heavy chain variable region amino acid sequence of SEQ ID NO: 1.

Disclosed herein are isolated antibodies comprising a light chain variable region amino acid sequence of SEQ ID NO: 13 and a heavy chain variable region amino acid sequence of SEQ ID NO: 12.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 9; a determining region light chain 2 (CDRL2) amino acid sequence of SEQ ID NO: 10; and a determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 17; a determining region light chain 2 (CDRL2) amino acid sequence of SEQ ID NO: 18; and a determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 2 and a heavy chain variable region amino acid sequence of SEQ ID NO: 1, wherein the isolated antibody comprises 1, 2, 3, 4, or 5 conservative amino acid substitutions in the light or heavy chain variable region amino acid sequences.

Disclosed herein are isolated antibodies comprising a light chain variable region amino acid sequence of SEQ ID NO: 13 and a heavy chain variable region amino acid sequence of SEQ ID NO: 12, wherein the isolated antibody comprises 1, 2, 3, 4, or 5 conservative amino acid substitutions in the light or heavy chain variable region amino acid sequences.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Other aspects of the invention are discussed throughout this application. Any aspect discussed with respect to one aspect of the invention applies to other aspects of the invention as well and vice versa. Each aspect described herein is understood to be aspects of the invention that are applicable to other aspects of the invention. It is contemplated that any aspect discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-G show that tau binds to poly(A) RNA, inhibiting MT assembly and promoting tau oligomerization. FIG. 1A shows that in vitro MT assembly detected by light scattering measured by absorbance at 340 nm expressed in absorbance units (AU). Tau initiates MT assembly under standard conditions (20 μM tubulin dimer in 1×BRB80 with 2 μM recombinant tau). Poly(A) RNA delays and attenuates MT assembly at equivalent tau concentrations in a poly(A) RNA concentration dependent manner (fold poly(A) levels relative to tau levels shown). Dashed lines represent SEM for n=5 replicates for each condition. FIG. 1B shows a representative data trace for a biotin poly(A) 20 RNA probe. SPR assay can detect tau/RNA interactions including association and dissociation to derive the parameters of binding kinetics (colored lines represent decreasing tau concentrations SPR in binding assay: teal-250 nM tau, yellow-200 nM tau, blue-150 nM tau, green-100 nM tau, red-50 nM tau, black-0 nM tau. FIG. 1C shows affinity measurements determined by SPR analysis for tau affinity with distinct biotinylated probes as indicated. Note that biotinylated tubulin does not bind poly(A) RNA detectably by SPR. FIG. 1D shows that incubation of recombinant purified tau protein and poly(A) RNA produces RNA/protein complexes detected by size exclusion chromatography (SEC). An equivalent mass of tau protein was fractionated by SEC on a Superdex 200 increase column. FIG. 1E shows bigenic poly(A)₄₅/tau transgenic animals (strain CK2408) exhibit more severe neuronal dysfunction as detected by swimming reflex measurements when compared to the parental tau transgenic strain (CK144) alone [mean±SEM: Tau Tg=0.098±0.006, poly(A)₄₅/Tau Tg=0.040±0.007, Student's t-test p<0.0001]. FIG. 1F shows that the poly(A)₄₅ RNA transgene increases pathological tau protein accumulation commensurate with behavioral dysfunction. FIG. 1G shows that the quantification of expression of total tau (DAKO) relative to tubulin load control, pS202 (CP13) relative to total tau, and pS396/404 (PHF1) relative to total in parental tau transgenic strain (CK144) as compared to tau/poly(A) coexpressing animals [error bars show SEM of three replicate blots. Student's t-test p=0.0294, 0.77, and 0.27, respectively].

FIGS. 2A-D shows the generation and validation of mAb TRC35 that recognizes tau/RNA complexes. FIG. 2A shows the workflow for generating mAb TRC35. FIG. 2B shows that mAb TRC35 recognizes a tau epitope enriched in Alzheimer's disease. Shown is a native protein dot blot loaded with equivalent mass of E. coli derived recombinant tau (top row, 20 ng) or Alzheimer's disease derived tau (ADtau) (bottom row, 20 ng). FIG. 2C shows the densitometry analysis of the dot blots from panel B reveal a statistically significant 8-fold selectivity of TRC35 for ADtau over native tau (p<0.0001, statistical comparison made by two-tailed Student's t-test). FIG. 2D shows that immunostaining of human tau transgenic mouse models of tauopathy show prominent phospho-tau (AT180) and TRC35+ labeling in both 9 month old PS19 mice, which exhibit neurofibrillary degeneration as the disease progresses, and 3 month old Tg2652 mice, which do not exhibit any fibrillary tau deposits. C57BL/6 (B6) mouse immunostaining included as controls. Insets depict TRC35 immunoreactivity at higher magnification in the CA1. Scale bar=500 um at low power magnification and 50 μm in the insets.

FIGS. 3A-C shows that MSUT2 influences TRC35 immunoreactivity in PS19 tauopathy mice. FIG. 3A shows that nine-month human tau transgenic PS19 tauopathy mice exhibit abundant tau lesions labeled with TRC35 accumulating in the stratum lacunosum moleculare (SLM) (arrow). FIG. 3B shows that PS19/MSUT2 KO mice exhibit decreased accumulation of TRC35 immunoreactivity in the SLM (arrow). Insets depict TRC35 immunoreactivity at higher magnification in the SLM. FIG. 3C shows that measurement of TRC35+ immunoreactivity in the SLM by quantitative digital microscopy demonstrates ˜4-fold decrease in TRC35+ immunoreactivity in MSUT2 KO mice (*** p<0.0001., statistical comparison made by two-tailed Student's t-test). Scale bar=50 μm at low power magnification and 100 μm in the insets.

FIGS. 4A-G shows that TRC35 labels abundant tau species in Alzheimer's disease. FIGS. 4A, 4D) show that TRC35 immunostaining of normal control autopsy brain tissue reveals diffuse neuronal soma staining in the frontal cortex, with no neuritic or fibrillary neuropathology. FIGS. 4B, 4C, 4E, 4F) show that TRC35 immunostaining in the frontal cortex of Alzheimer's disease cases revealed two distinct pathological groups: cases with modest cytoplasmic staining and sporadic regions of neuritic pathology (FIGS. 4B, 4E) and cases with extensive fibrillar immunoreactivity within neuronal soma as well as dystrophic neurites and neuropil threads (FIGS. 4C, 4F). FIGS. 4D and 4F show higher magnifications of cortical staining shown in FIGS. 4A-C. Scale bar=100 μm A-C and 50 μm D-F. FIG. 4G shows the measurement of TRC35 immunoreactivity in cases (N=19) and controls (N=5) (statistical comparison made by student's T-test, p=0.0124).

FIGS. 5A-C show the regional distribution of TRC35 immunoreactivity in AD. FIG. 5A show that TRC35 immunostaining of hippocampus sections from Alzheimer's disease autopsy brain reveal abundant TRC35 immunoreactivity. FIG. 5B show that TRC35 immunostaining of amygdala sections from Alzheimer's disease autopsy brain reveal abundant TRC35 immunoreactivity. Asterisks indicate the region depicted at higher magnification in the insets in FIG. 5A and FIG. 5B. FIG. 5C show that TRC35 immunostaining of cerebellum sections from Alzheimer's disease autopsy brain reveal an absence of TRC35 immunoreactivity. Scale bar=500 μm in low power images and 50 μm in insets.

FIGS. 6A-F show that diverse tauopathy disorders exhibit TRC35 immunoreactivity. FIG. 6A show that progressive supranuclear palsy cases exhibit TRC35+ NFTs (arrow) and tufted astrocytes (arrowhead) in grey matter. FIG. 6B show that progressive supranuclear palsy cases also have TRC35+ oliogdendrogial coils (arrowhead) in white matter. FIG. 6C show that corticobasal degeneration cases exhibited TRC35+ neuronal soma (arrowhead) and neuropil threads in grey matter. FIG. 6D show that corticobasal degeneration cases also exhibit abundant white matter pathology. FIG. 6E show that Pick's disease cases exhibit spherical TRC35+ labeled Pick bodies (arrow). FIG. 6F show that the frontal cortex section from brain autopsy of an oculopharyngeal muscular dystrophy case exhibits TRC35 pathological tau deposition including neuropil threads and NFTs (arrowhead). Scale bar=50 μm.

FIGS. 7A-G show the interaction between TRC35 and PABPN1. Alzheimer's disease cases with normal cortical PABPN1 immunoreactivity (FIG. 7A) exhibited more modest TRC35 immunoreactivity characterized by sparse neuritic immunoreactivity and sporadic NFTs (FIG. 7C). In contrast, Alzheimer's disease cases with PABPN1 depletion in the frontal cortex (FIG. 7B) exhibited more severe accumulation of pathological tau as measured by TRC35 immunostaining that included abundant apparent NFTs and dystrophic neurite profiles (FIG. 7D). FIG. 7E shows the densitometry analysis of TRC35 positive immunoreactivity in PABPN1-positive Alzheimer's disease cases (n=11) compared to PABPN1-depleted Alzheimer's disease cases (n=8) (** p=0.001 by two-tailed Student's t-test). Scale bar=100 μm. FIG. 7F shows the dual label immunofluorescence for PABPN1 (red) and TRC35 (green) in Alzheimer's disease cases. FIG. 7G shows that TRC35 immunostaining was quantitated and graphed with the age of onset for each Alzheimer's disease case analyzed (N=19). TRC35 immunoreactivity correlated with the age of disease onset in Alzheimer's disease (Pearson correlation coefficient=0.59, P=0.007).

FIG. 8 shows the role of RNA binding in tau neuropathology and MT function; illustrating the tau/RNA interaction with MT function and tau neuropathology. Previous work has shown tau binds poly(A) RNA26. Under normal conditions, RNA binding proteins shield poly(A) RNA and there is little exposed poly(A) RNA for tau to bind. Under pathological conditions, poly(A) RNA binding proteins are lost and poly(A) RNA becomes exposed. Poly(A) RNA has a higher affinity for tau than does tubulin dimers and RNA seeds tau aggregation leading to both tau loss of function from MTs and tau TRC formation.

FIGS. 9A-B show dot blots of recombinant and Alzheimer's disease derived human tau. To control for pathological vs non-pathological features of tau, recombinant protein was purified from E. coli overexpressing human tau and from human Alzheimer's disease brain. FIG. 9A shows that 40 ng of purified tau proteins were dot blotted in 16 replicate samples and dot blotted as described in FIG. 1 using SP70 to control for total tau and pS422 to measure pathological (pTau). Recombinant samples contain similar amounts of total tau as Alzheimer's disease brain purified tau. However, pTau is absent from recombinant material, while abundant in Alzheimer's disease brain derived tau. FIG. 9B shows that 20 ng of Alzheimer's disease derived fibrillar tau proteins from six different Alzheimer's disease cases were dot blotted in 5 replicate spots and probed with mAb TRC35 as above.

FIGS. 10A-C show the evaluation of TRC reactivity in Alzheimer's disease and control brain lysates. To control for pathological vs non-pathological features of tau, total brain lysates were extracted from human postmortem brain specimens. Five μg of total protein were dot blotted in 5 replicate samples per case (N=5 Alzheimer's disease cases and age matched controls) using TRC35 as the primary antibody (FIG. 10A) and total tau (FIG. 10B). FIG. 10C shows that the measurement of TRC35 dot blot immunoreactivity significantly increased TRC35 signal (p<0.0001).

FIGS. 11A-C show that Poly(A) RNA induces TRC35 reactivity conformation better than Heparin. To control for other polyanions inducing the TRC35+ tau conformation, recombinant protein was purified from E. coli overexpressing human tau. Twenty ng of purified tau proteins was incubated with poly(A) RNA or Heparin as shown in representative blots above. In total, 8 replicate samples were dot blotted with mAb TRC35 (FIG. 11A) and SP70 total tau mAb (FIG. 11B) as previously described for FIG. 1. FIG. 11C) Measurement of TRC35 signal and normalization to SP70 shows ˜2-fold more TRC35+ tau conformation in the poly(A) incubated tau sample relative to heparin (p=0.020).

FIG. 12 shows the extensive colocalization between monomeric tau and TRC35+ tau in differentiated ReN VM Cells. Representative images are displayed as split channels, merged, the colocalization of the two species, and the plot of the Pearson Coefficient of Correlation of the two species (CLC=0.6). Immunostaining includes TRC35+ (RNA induced conformational/oligomeric specific tau—green in merged image) and SP70 (monomeric tau—red in merged image). DAPI labels nuclei (blue in merged image). Scale bar=25 μm.

FIG. 13 shows a summary map of mAb epitope analysis. Summary of peptide array analysis from Table 2 mapped onto primary amino acid tau sequence. SEQ ID NO: 20 is shown.

FIGS. 14A-B show that PABPN1 disrupts high molecular weight TRC oligomers. Recombinant tau protein was incubated with poly(A) RNA and resolved by size exclusion chromatography as described for FIG. 1D. Incubations occurred with or without PABPN1 at approximately equal molar concentration to tau. Note that the ˜700 kDa fraction contains detectable tau RNA complexes including high molecular weight multimeric tau species (shown in red bracket in FIG. 14A. FIG. 14B shows that these higher molecular weight multimeric species are not observed. Also note peak RNA containing fractions are noted between FIG. 14A and FIG. 14B.

FIGS. 15A-F show Extensive tau neuropathology in an ocular pharyngeal muscular dystrophy (OPMD) case. Evaluation of pTau accumulation in OPMD brain. AT8 immunostaining revealed robust phospho-tau in neurons, neurites and neuropil threads in multiple brain regions, including frontal cortex (FIG. 15A), temporal cortex (FIG. 15B), hippocampus (FIG. 15C), substantia nigra (FIG. 15D), and striatum (FIG. 15E). In contrast, the cerebellum was negative (FIG. 15F). Scale bar=50 μm. Secondary antibody is goat anti-mouse.

FIG. 16 shows peptide array epitope mapping against 11 mer tau peptides. Shown is a comparison of TRC35 with other related tau mouse mAbs that have been previously characterized in a quantitative JPT peptide array. Note that human and mouse IgG serve as positive and negative controls, respectively, for array assay.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. “Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

“Treatment” and “treating” refer to administration or application of a therapeutic agent (e.g., a TRC35 antibody described herein) to a subject or performance of a procedure or modality on a subject for the purpose of obtaining a therapeutic benefit of a disease or health-related condition. For example, a treatment may include administration of a pharmaceutically effective amount of an antibody that binds to aggregated tau/RNA complexes.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a neurodegenerative disease or disorder, a tauopathy, dementia or ocular pharyngeal muscular dystrophy.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment, such as, for example, prior to the administering step.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂ (CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)ₙ can be used (n=1, 2, 3 or 4).

A “fragment antigen-binding fragment (Fab)” is a region of an antibody that binds to antigen. An Fab comprises constant and variable regions from both heavy and light chains.

A “CDR” or complementarity determining region is a region of hypervariability interspersed within regions that are more conserved, termed “framework regions” (FR).

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256:495-497, 1975), or by recombinant DNA methods.

The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al. (1984), infra; Morrison et al., Proc. Natl. Acad. Sci. U.S.A. 81:6851).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033).

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein the terms “amino acid” and “amino acid identity” refers to one of the naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the(S) configuration. In some aspects, the amino acids are in the(S) or L-configuration. If non-naturally occurring side chains are used, non-amino acid substituents may be used, for example to prevent or retard in vivo degradation.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Alzheimer's disease and related disorders are associated with neurofibrillary tangles and other neuropathological lesions composed of detergent-insoluble tau protein. In recent structural biology studies of tau proteinopathy, aggregated tau forms a distinct set of conformational variants specific to the different types of tauopathy disorders. However, the constituents driving the formation of distinct pathological tau conformations on pathway to tau-mediated neurodegeneration remain unknown. Previous work demonstrated RNA can serve as a driver of tau aggregation, and RNA associates with tau containing lesions, but tools for evaluating tau/RNA interactions remain limited.

Pathological tau deposits in the form of neurofibrillary tangles (NFTs) have long been appreciated as a potential driver of neurodegeneration in Alzheimer's disease. NFTs have been shown to strongly correlate with cognitive impairment in Alzheimer's disease, in contrast to β-amyloid plaques, which do not consistently correlate with dementia (Hyman B T and Gomez-Isla T. Neurobiol Aging. July-August 1997; 18 (4): 386-7; discussion 389-92; Gomez-Isla T, et al. Ann Neurol. January 1997; 41 (1): 17-24; and Arriagada P V, et al. Neurology. March 1992; 42 (3 Pt 1): 631-9). In other types of dementia disorders, termed tauopathies, tau is the primary neuropathological lesion. Likewise, imaging studies support a causal relationship between abnormal tau and dementia (Holtzman D M, et al. Alzheimers Dement. October 2016; 12 (10): 1033-1039). However, the genesis of abnormal tau and the molecular mechanisms by which pathological tau contributes to neurodegeneration remains incompletely understood (Ballatore C, et al. Nat Rev Neurosci. September 2007; 8 (9): 663-72). The canonical biochemical function of tau is that of a microtubule (MT) binding protein, as it was first identified to both bind to MTs and promote their polymerization (Weingarten M D, et al. Proc Natl Acad Sci USA. May 1975; 72 (5): 1858-62). More recently, tau has been shown to interact with a wide range of macromolecules, perhaps due to its unusual natively unfolded structure (reviewed in (Limorenko G and Lashuel H A. Chem Soc Rev. Dec. 10 2021)). Indeed, the conversion of natively unfolded tau into abnormal misfolded fibrillar structures has been an intense area of inquiry, with the recent determination of the fibril core structure for many tauopathies by cryoelectron microscopy (reviewed in (Goedert M. Essays Biochem. Dec. 22 2021; 65 (7): 949-959; and Scheres S H, et al. Curr Opin Struct Biol. October 2020; 64:17-25)). The structural determination of the distinct conformers of abnormal tau fibrils has validated the tauopathy seeding/strain hypothesis by demonstrating that differing diseases exhibit structurally distinct tau aggregates contributing to disease (Zhang W, et al. Nature. April 2020; 580 (7802): 283-287; Falcon B, et al. Nature. September 2018; 561 (7721): 137-140; and Fitzpatrick A W P, et al. Nature. Jul. 13 2017; 547 (7662): 185-190). How structurally distinct tau aggregated species arise remains an open question, but different tau interacting macromolecules have been suggested to play an important role (Wegmann S, et al. EMBO J. Apr. 3 2018; 37 (7); Eftekharzadch B, et al. Neuron. Sep. 5 2018; 99 (5): 925-940 c7; Wang Y and Mandelkow E. Nat Rev Neurosci. January 2016; 17 (1): 5-21; Kampers T, et al. FEBS Letters. Dec. 16, 1996 1996; 399 (3): 344-349; Zhang X, et al. PLOS Biol. July 2017; 15 (7): c2002183; Crowe A, et al. Biochem Biophys Res Commun. Jun. 22 2007; 358 (1): 1-6; and Barghorn S, Mandelkow E. Biochemistry. Dec. 17 2002; 41 (50): 14885-96).

A wide variety of polyanions stimulate tau aggregation in vitro, including heparin, RNA, polyglutamate, and fatty acids; tau also binds the highly negatively charged c-terminal tail of alpha tubulin (helix 12) to promote MT polymerization and stabilization (Al-Bassam J, et al. J Cell Biol. Jun. 24 2002; 157 (7): 1187-96). A previous study suggests that RNA is the most potent polyanion trigger of tau aggregation in vitro (Kampers T, et al. FEBS Letters. Dec. 16, 1996 1996; 399 (3): 344-349). Of the tau aggregation stimulatory polyanions mentioned above, RNA appears to be the most abundant in the neuronal cytoplasm. Further, neuropathological and macromolecular characterization of Alzheimer's disease brain NFTs contain RNA; analysis of Alzheimer's disease-derived NFTs by microarray based transcriptomic profiling showed certain mRNAs preferentially become trapped in tau aggregates in human disease (Ginsberg S D, et al. Ann Neurol. February 1997; 41 (2): 200-9; Ginsberg S D, et al. Acta Neuropathologica. November 1998 1998; 96 (5): 487-494; and Ginsberg S D, et al. Ann Neurol. July 2000; 48 (1): 77-87). Next generation sequencing based transcriptomic analysis has confirmed these findings and extended them to cellular and transgenic animal models of tau aggregation, as well as progressive supranuclear palsy (PSP) and corticobasal degeneration (CBD) (Lester E, et al. Neuron. Apr. 7 2021; and McMillan P J, et al. Jun. 29 2021; 9 (1): 117). Surprisingly, tau's MT-stabilizing activity has also been shown to be impacted by RNA in experiments where RNA homopolymers blocked tau mediated MT assembly (Bryan J B, et al. Proc Natl Acad Sci USA. September 1975; 72 (9): 3570-4).

In previous work, RNA binding protein modulators of tauopathy that reside in nuclear speckles were identified, including aly-1,2,3/ALYREF, pabp-2/PABPN1, sut-1, sut-2/MSUT2, and parn-2/TOE1 (Kow R L, et al. Geroscience. Feb. 4 2022; doi: 10.1007/s11357-022-00526-2; Kow R L, et al. Neurobiol Dis. January 2021; 147:105148; Wheeler J M, et al. Sci Transl Med. Dec. 18 2019; 11 (523); Guthrie C R, et al. Hum Mol Genet. May 15 2011; 20 (10): 1989-99; Guthrie C R, et al. Hum Mol Genet. May 15 2009; 18 (10): 1825-38; Kraemer B C and Schellenberg G D. Hum Mol Genet. 2007 Aug. 15 2007; 16 (16): 1959-71). Among these RNA binding proteins, we have shown that MSUT2 controls tauopathy related phenotypes in brain neurons in mammals. MSUT2 knockout mice exhibit reduced accumulation of pathological tau, cognitive impairment, and neurodegeneration (Wheeler J M, et al. Sci Transl Med. Dec. 18 2019; 11 (523). The molecular mechanism of MSUT2 modulation of tauopathy involves the nuclear RNA binding functions of MSUT2 as it binds both poly(A) RNA and the nuclear poly(A) RNA binding protein, PABPN1. The data showed that MSUT2 and PABPN1 co-localize with poly(A) RNA to nuclear speckles, forming a macromolecular complex. In the brains of post-mortem Alzheimer's disease cases both MSUT2 and PABPN1 become co-depleted. Work of others showed PABPN1 and MSUT2 have opposing effects on mRNA poly(A) tail length (Kelly S M, et al. Dev Neurobiol. January 2016; 76 (1): 93-106; and Kelly S M, et al. RNA. May 2014; 20 (5): 681-8). Likewise, it was observed that MSUT2 and PABPN1 function together in a reciprocal fashion to influence tauopathy; normal MSUT2 function drives tau aggregation while normal PAPBN1 function promotes tau proteostasis.

Disclosed herein are the results of molecular interaction studies carried out to measure the impact of tau/RNA binding on tau microtubule binding and aggregation. To investigate the importance of tau/RNA complexes (TRCs) in neurodegenerative disease, a monoclonal antibody (mAb TRC35 also referred herein as “TRC35”) was raised against aggregated tau/RNA complexes. The results show that native tau binds RNA with high affinity but low specificity, and tau binding to RNA competes with tau-mediated microtubule assembly functions. Tau/RNA interaction in vitro promotes the formation of higher molecular weight tau/RNA complexes, which represent an oligomeric tau species. Co-expression of tau and poly(A)₄₅ RNA transgenes in Caenorhabditis elegans exacerbates tau related phenotypes including neuronal dysfunction and pathological tau accumulation. TRC35 exhibits specificity for Alzheimer's disease-derived detergent insoluble tau relative to soluble recombinant tau. Immunostaining with TRC35 labels a wide variety of pathological tau lesions in animal models of tauopathy, which are reduced in mice lacking the RNA binding protein MSUT2. TRC-positive lesions are evident in many human tauopathies including Alzheimer's disease, progressive supranuclear palsy, corticobasal degeneration, and Pick's disease. Ocular pharyngeal muscular dystrophy is also identified herein as a tauopathy disorder where loss of function in the poly(A) RNA binding protein (PABPN1) causes accumulation of pathological tau in tissue from postmortem human brain. Tau/RNA binding drives tau conformational change and aggregation inhibiting tau mediated microtubule assembly. The findings show that cellular tau/RNA interactions are modulators of both normal tau function and pathological tau toxicity in tauopathy disorders, and provide a therapeutic approach to targeting TRCs.

### Antibodies

Disclosed herein are antibodies that can bind to tau/RNA complexes. In some aspects, the antibodies can bind to human aggregated tau/RNA complexes. In some aspects, the antibodies disclosed herein can be isolated antibodies. Examples of the CDR sequences and heavy or light chain variable region sequences of the disclosed antibodies (e.g., TRC35 and TRC1) are shown in Table 2.

An example of identifying and isolating a monoclonal antibody is described below.

The term “CDR” as used herein refers to a Complementarity Determining Region of an antibody variable domain. Systematic identification of residues included in the CDRs have been developed by Kabat et al. (1991, Sequences of Proteins of Immunological Interest, 5th Ed., United States Public Health Service, National Institutes of Health, Bethesda). Variable light chain (VL) CDRs are herein defined to include residues at positions 27-32 (CDR1), 50-56 (CDR2), and 91-97 (CDR3). Variable heavy chain (VH) CDRs are herein defined to include residues at positions 27-33 (CDR1), 52-56 (CDR2), and 95-102 (CDR3).

Additionally, a universal numbering system has been developed and widely adopted, ImMunoGeneTics (IMGT) Information System® (Lafranc et al., 2003, Dev. Comp. Immunol., 27 (1): 55-77). IMGT is an integrated information system specializing in immunoglobulins (Ig), T cell receptors (TR) and the major histocompatibility complex (MHC) of human and other vertebrates. Herein, the CDRs are referred to in terms of both the amino acid sequence and the location within the light or heavy chain. As the “location” of the CDRs within the structure of the immunoglobulin V domain is conserved between species and present in structures called loops, by using numbering systems that align variable domain sequences according to structural features, CDR and framework residues and are readily identified. This information can be used in grafting and in the replacement of CDR residues from immunoglobulins of one species into an acceptor framework from, typically, a human antibody. An additional numbering system (AHon) has been developed by Honegger et al., 2001, J. Mol. Biol., 309:657-670. Correspondence between the numbering system, including, for example, the Kabat numbering and the IMGT unique numbering system, is well known to one skilled in the art (see, e.g., Kabat, Id; Chothia et al., Id.; Martin, 2010, Antibody Engineering, Vol. 2, Chapter 3, Springer Verlag; and Lefranc et al., 1999, Nuc. Acids Res., 27:209-212).

CDR region sequences have also been defined by AbM, Contact and IMGT. The AbM hypervariable regions represent a compromise between the Kabat CDRs and Chothia structural loops, and are used by Oxford Molecular's AbM antibody modeling software (see, e.g., Martin, 2010, Antibody Engineering, Vol. 2, Chapter 3, Springer Verlag). The “contact” hypervariable regions are based on an analysis of the available complex crystal structures. The residues from each of these hypervariable regions or CDRs are noted below in Table 1.

Exemplary delineations of CDR region sequences are illustrated in Table 2. The positions of CDRs within a canonical antibody variable region have been determined by comparison of numerous structures (Al-Lazikani et al., 1997, J. Mol. Biol., 273:927-948); Morea et al., 2000, Methods, 20:267-279). Because the number of residues within a hypervariable region varies in different antibodies, additional residues relative to the canonical positions are conventionally numbered with a, b, c and so forth next to the residue number in the canonical variable region numbering scheme (Al-Lazikani et al., Id). Such nomenclature is similarly well known to those skilled in the art.

TABLE 1
Exemplary Delineations of CDR Region Sequences (amino
acid positions reflected in each column).

| | IMGT | Kabat |
|---|---|---|
| V_{H} CDR1 | 28-35 | 31-35 |
| V_{H} CDR2 | 51-58 | 47-56 |
| V_{H} CDR3 | 97-103 | 99-103 |
| V_{L} CDR1 | 27-32 | 24-34 |
| V_{L} CDR2 | 50-52 | 50-56 |
| V_{L} CDR3 | 89-97 | 89-97 |

In some aspects, the CDRs of the disclosed antibodies can be defined according to the Kabat numbering system. In some aspects, the CDRs of the disclosed antibodies can be defined according to the IMGT numbering system.

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3 or 4 amino acid changes as compared to the parent or reference CDR of the invention, and shares or improves biological function, specificity and/or activity of the parent CDR.

In some aspects, the antibody can be an IgG class of antibody, wherein the IgG class antibody can be an IgG1, IgG2, IgG3, or IgG4 class antibody. In some aspects, the antibody can comprise a VH amino acid sequence at least 90% identical to the sequences disclosed in Table 2 and/or a VL amino acid sequence at least 90% identical to the sequences disclosed in Table 2. In some aspects, the antibody comprises a VH amino acid sequence according to the sequences disclosed in Table 2 and/or a VL amino acid sequence according to the sequences disclosed in Table 2.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11. In some aspects, the heavy chain variable region can comprise a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Also disclosed herein, are isolated antibodies comprising a light chain variable region amino acid sequence of SEQ ID NO: 2 and a heavy chain variable region amino acid sequence of SEQ ID NO: 1. In some aspects, disclosed are isolated antibodies comprising a light chain variable region amino acid sequence of SEQ ID NO: 2 and a heavy chain variable region amino acid sequence of SEQ ID NO: 1, wherein the CDRs are the CDRs as provided by the Kabat numbering system. In some aspects, disclosed are isolated antibodies comprising a light chain variable region amino acid sequence of SEQ ID NO: 2 and a heavy chain variable region amino acid sequence of SEQ ID NO: 1, wherein the CDRs are the CDRs as provided by the IMGT numbering system.

In some aspects, any of the antibodies disclosed herein can comprise a light chain variable region amino acid sequence comprising SEQ ID NO: 2. In some aspects, any of the antibodies disclosed herein can comprise a heavy chain variable region amino acid sequence comprising SEQ ID NO: 1. In some aspects, a light chain variable region has an amino acid sequence that is at least 90% identical to amino acid sequence SEQ ID NO: 2. In some aspects, a heavy chain variable region has an amino acid sequence that is at least 90% identical to amino acid sequence SEQ ID NO: 1.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 9; a determining region light chain 2 (CDRL2) amino acid sequence of SEQ ID NO: 10; and a determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 2 and a heavy chain variable region amino acid sequence of SEQ ID NO: 1, wherein the isolated antibody comprises 1, 2, 3, 4, or 5 conservative amino acid substitutions in the light or heavy chain variable region amino acid sequences.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19. In some aspects, the heavy chain variable region can comprise a complementarity determining region heavy chain 1 (CDRH1) amino acid sequence of SEQ ID NO: 14; and a complementarity determining region heavy chain 2 (CDRH2) amino acid sequence of SEQ ID NO: 15.

In some aspects, the variable light chain (VL) CDRs are herein defined to include residues at positions 27-37 (CDR1), 55-57 (CDR2), and 94-100 (CDR3) of SEQ ID NO: 13. In some aspects, the variable heavy chain (VH) CDRs are herein defined to include residues at positions 26-33 (CDR1), 51-58 (CDR2), and 95-102 (CDR3) of SEQ ID NO: 12.

In some aspects, any of the CDR1, CDR2, or CDR3 of the variable light chain can be a single amino acid. In some aspects, any of the CDR1, CDR2, or CDR3 of the variable heavy chain can be a single amino acid. In some aspects, any of the antibodies disclosed herein can have not have a variable heavy chain CDR3.

Also disclosed herein, are isolated antibodies comprising a light chain variable region amino acid sequence of SEQ ID NO: 13 and a heavy chain variable region amino acid sequence of SEQ ID NO: 12. In some aspects, disclosed are isolated antibodies comprising a light chain variable region amino acid sequence of SEQ ID NO: 13 and a heavy chain variable region amino acid sequence of SEQ ID NO: 12, wherein the CDRs are the CDRs as provided by the Kabat numbering system. In some aspects, disclosed are isolated antibodies comprising a light chain variable region amino acid sequence of SEQ ID NO: 13 and a heavy chain variable region amino acid sequence of SEQ ID NO: 12, wherein the CDRs are the CDRs as provided by the IMGT numbering system.

In some aspects, any of the antibodies disclosed herein can comprise a light chain variable region amino acid sequence comprising SEQ ID NO: 13. In some aspects, any of the antibodies disclosed herein can comprise a heavy chain variable region amino acid sequence comprising SEQ ID NO: 12. In some aspects, a light chain variable region has an amino acid sequence that is at least 90% identical to amino acid sequence SEQ ID NO: 13. In some aspects, a heavy chain variable region has an amino acid sequence that is at least 90% identical to amino acid sequence SEQ ID NO: 12.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 17; a determining region light chain 2 (CDRL2) amino acid sequence of SEQ ID NO: 18; and a determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 13 and a heavy chain variable region amino acid sequence of SEQ ID NO: 12, wherein the isolated antibody comprises 1, 2, 3, 4, or 5 conservative amino acid substitutions in the light or heavy chain variable region amino acid sequences.

Disclosed herein are recombinant antibodies or fragments thereof that can bind to aggregated tau/RNA complexes. In some aspects, the antibody or fragment thereof can be a humanized antibody. In some aspects, the antibody can be an IgG, IgM, IgA, IgD, IgE, or a genetically modified IgG class antibody comprising any of the sequences disclosed in Table 2. In some aspects, the antibody can be an IgG class of antibody, wherein the IgG class antibody is an IgG1, IgG2, IgG3, or IgG4 class antibody. In some aspects, the antibody comprises a VH amino acid sequence at least 90% identical to a sequence disclosed in Table 2 or a fragment thereof and/or a VL amino acid sequence at least 90% identical to a sequence disclosed in Table 2 or a fragment thereof. In some aspects, the antibody comprises a VH amino acid sequence at least 90% identical to SEQ ID NO: 1 or a fragment thereof and/or a VL amino acid sequence at least 90% identical to SEQ ID NO: 2 or a fragment thereof. In some aspects, the antibody comprises a VH amino acid sequence at least 90% identical to SEQ ID NO: 12 or a fragment thereof and/or a VL amino acid sequence at least 90% identical to SEQ ID NO: 13 or a fragment thereof.

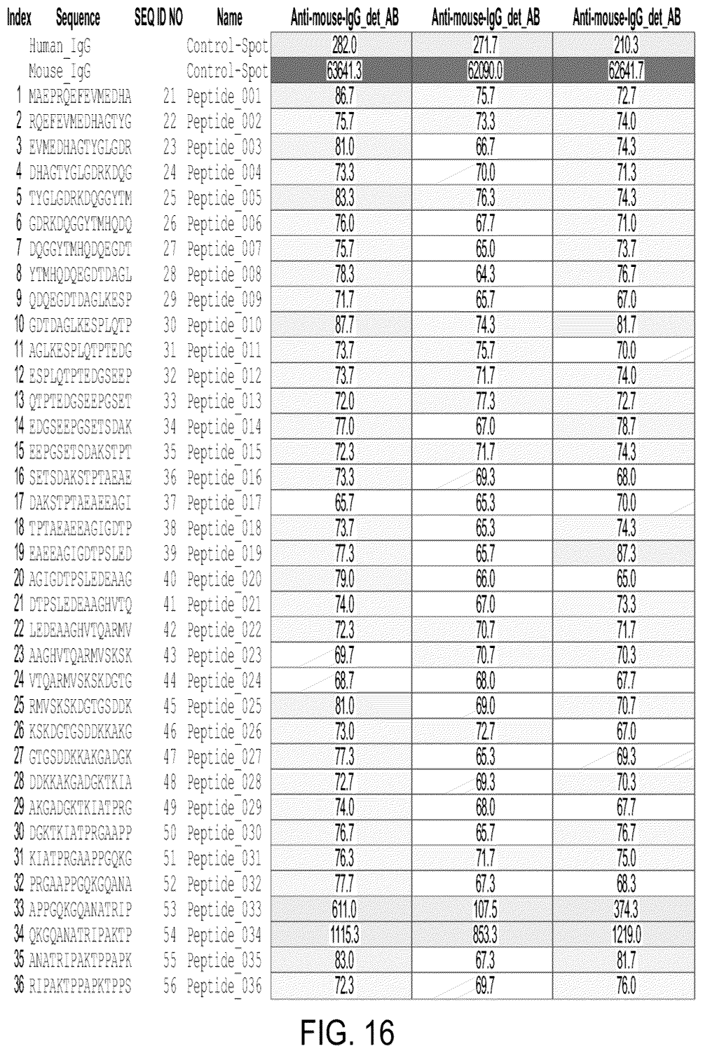

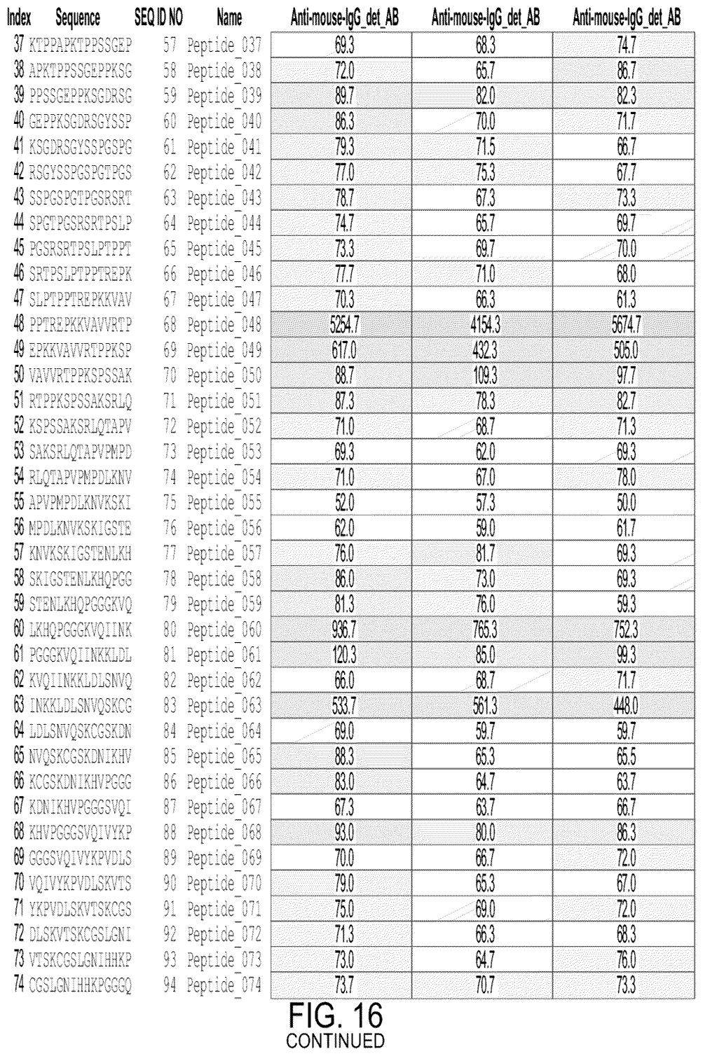

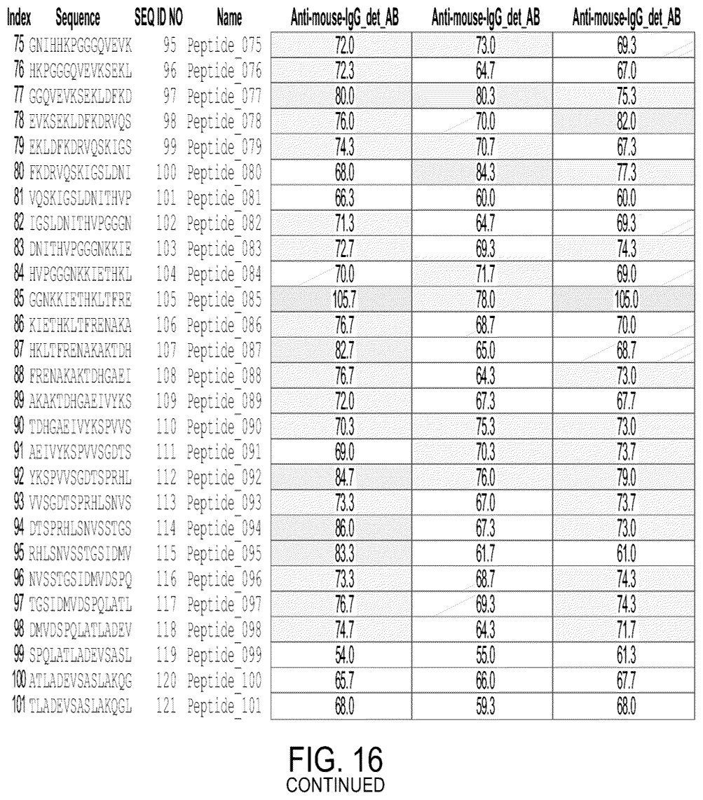

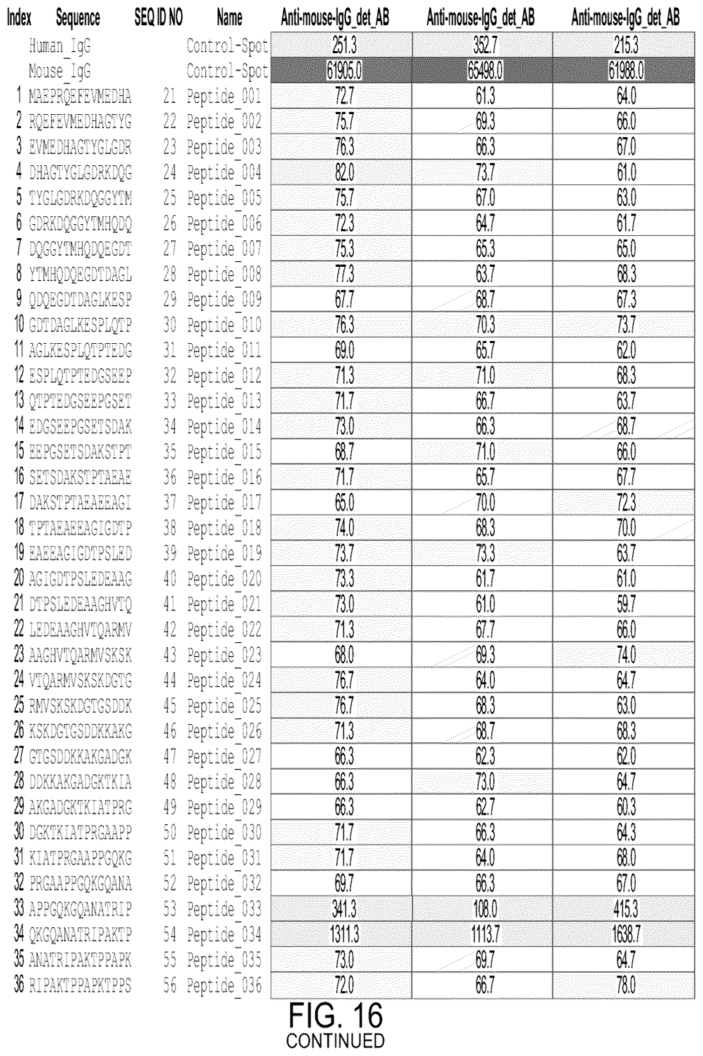

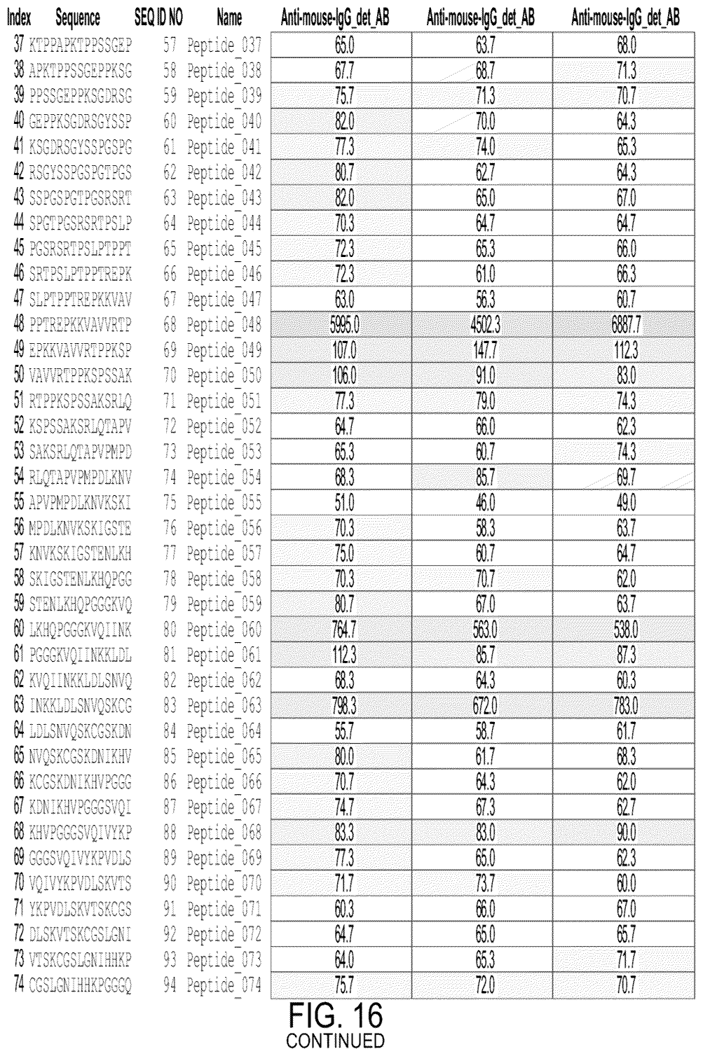

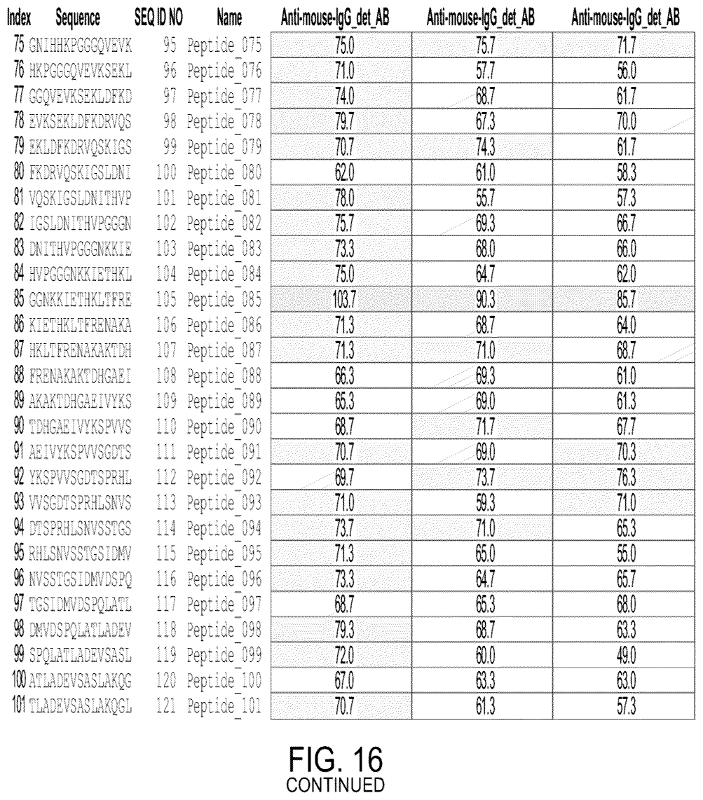

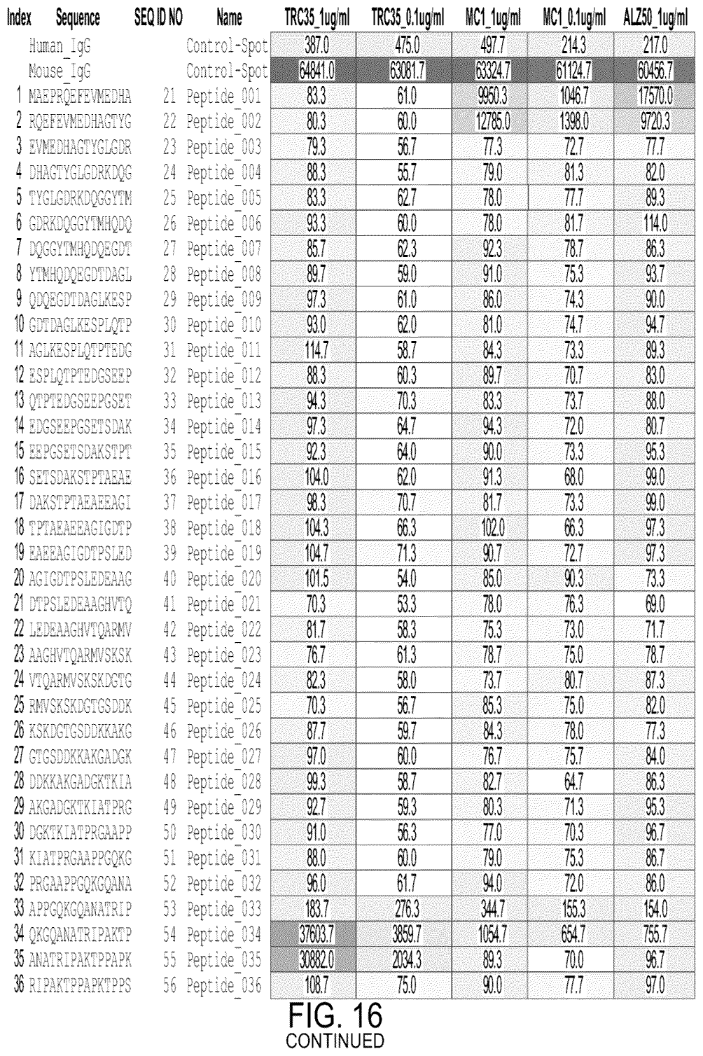

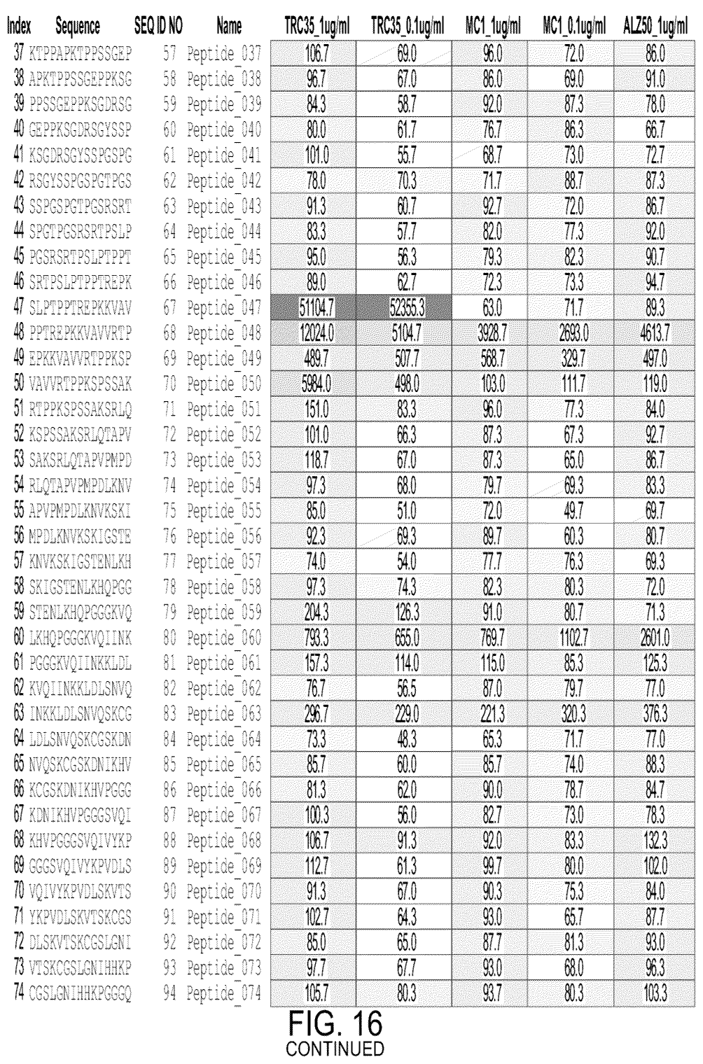

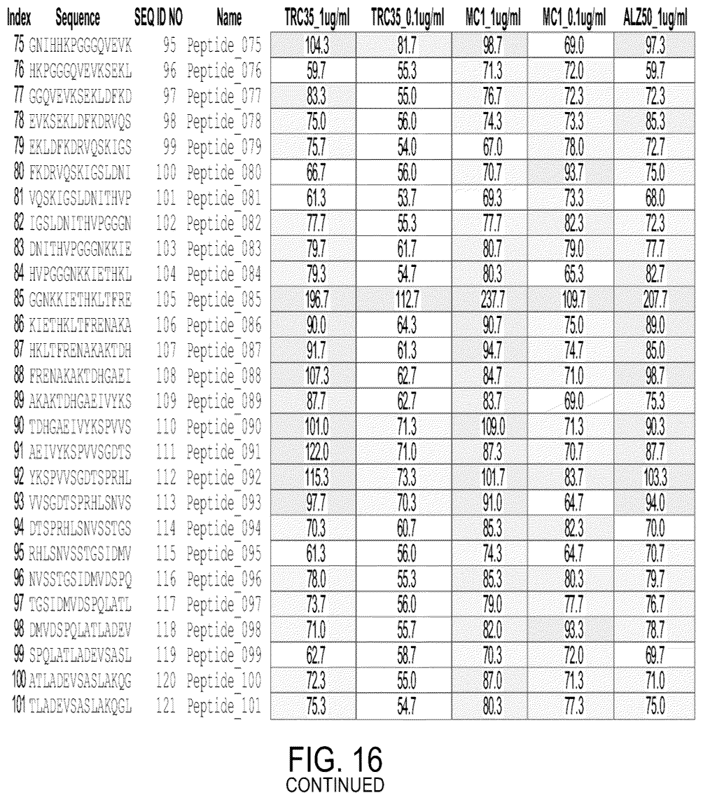

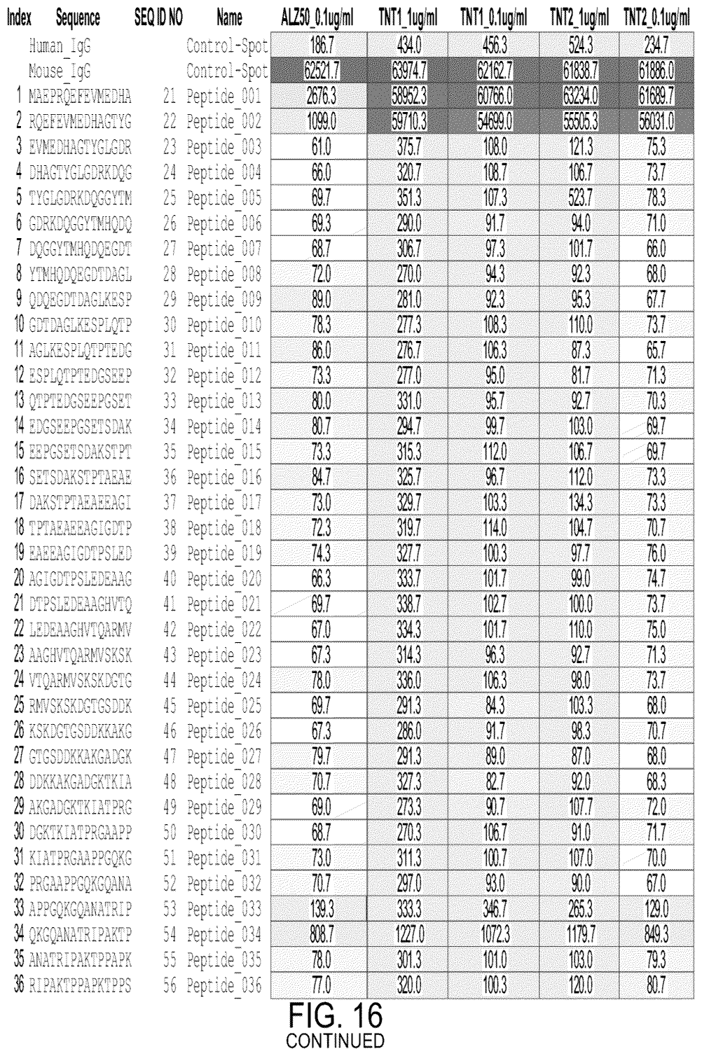

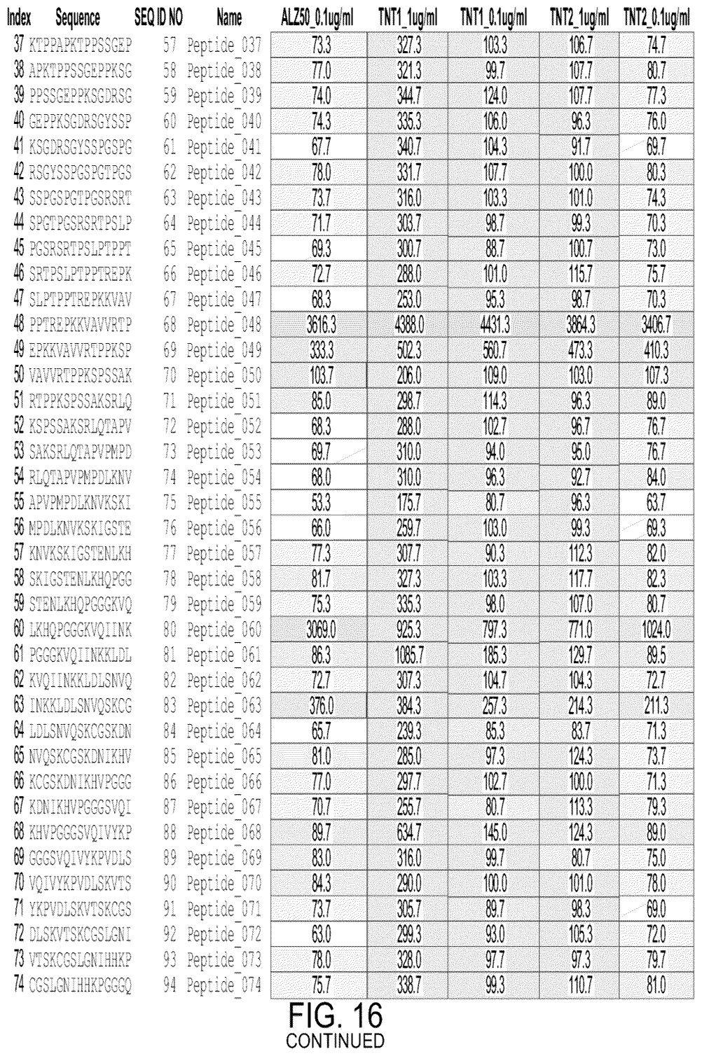

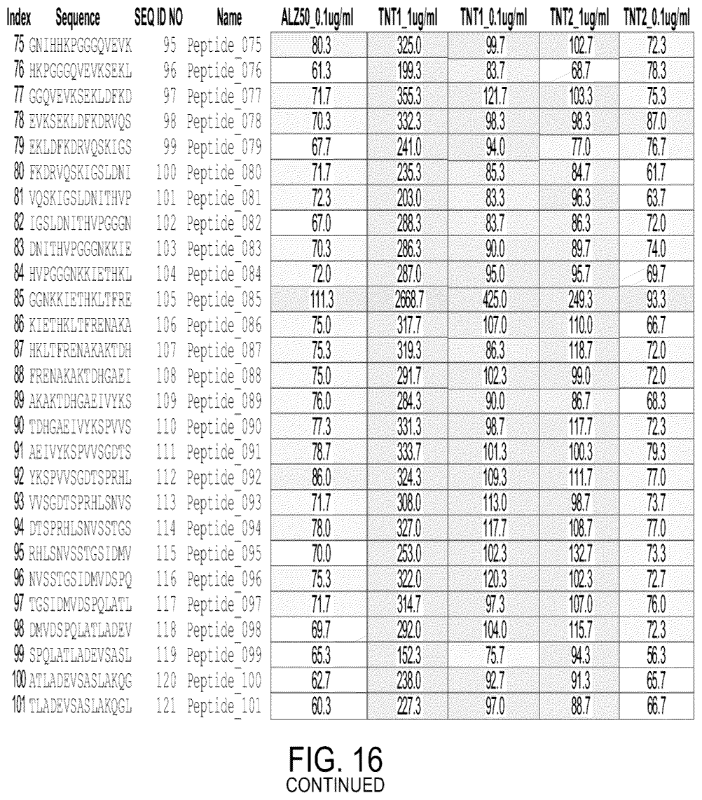

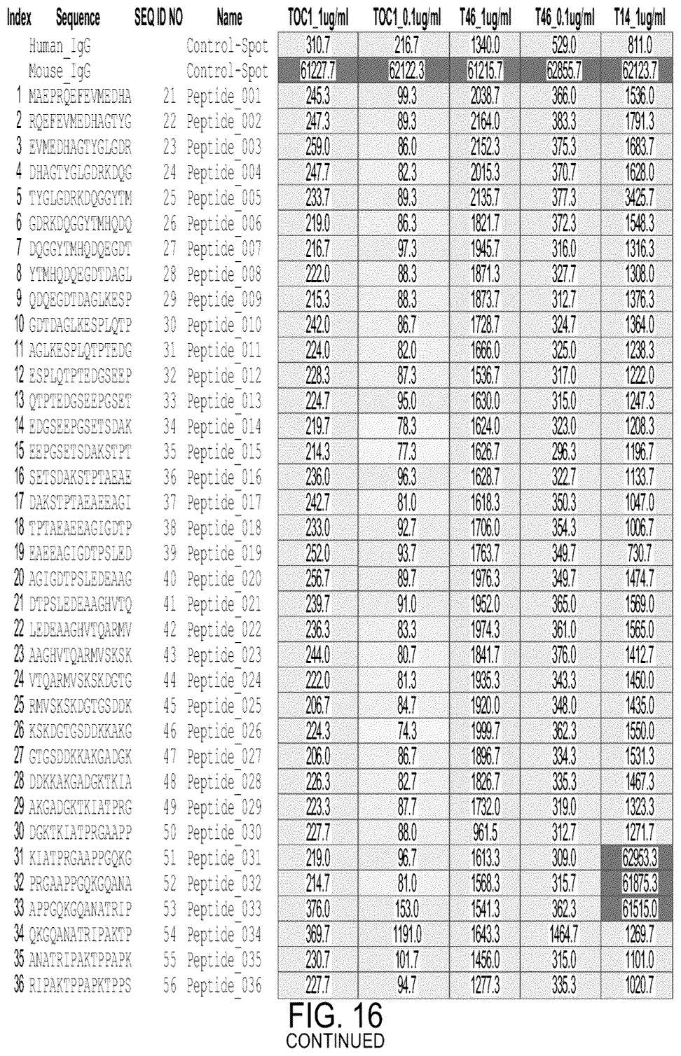

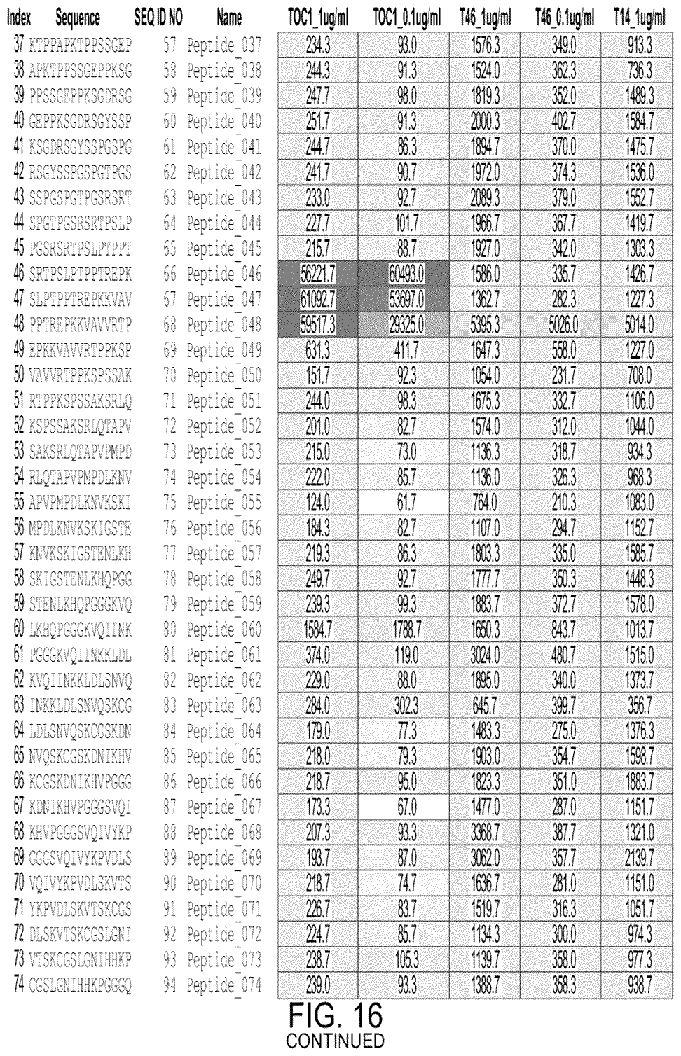

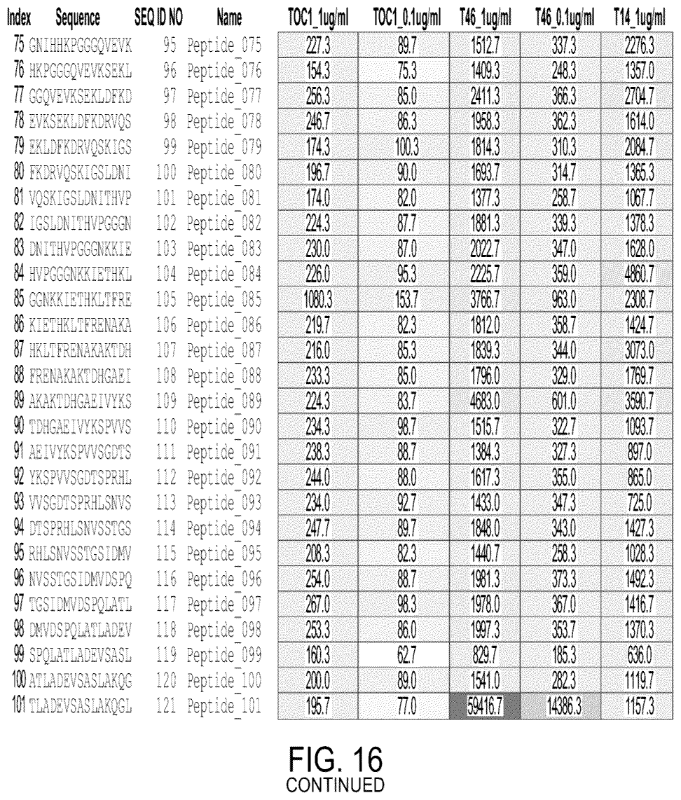

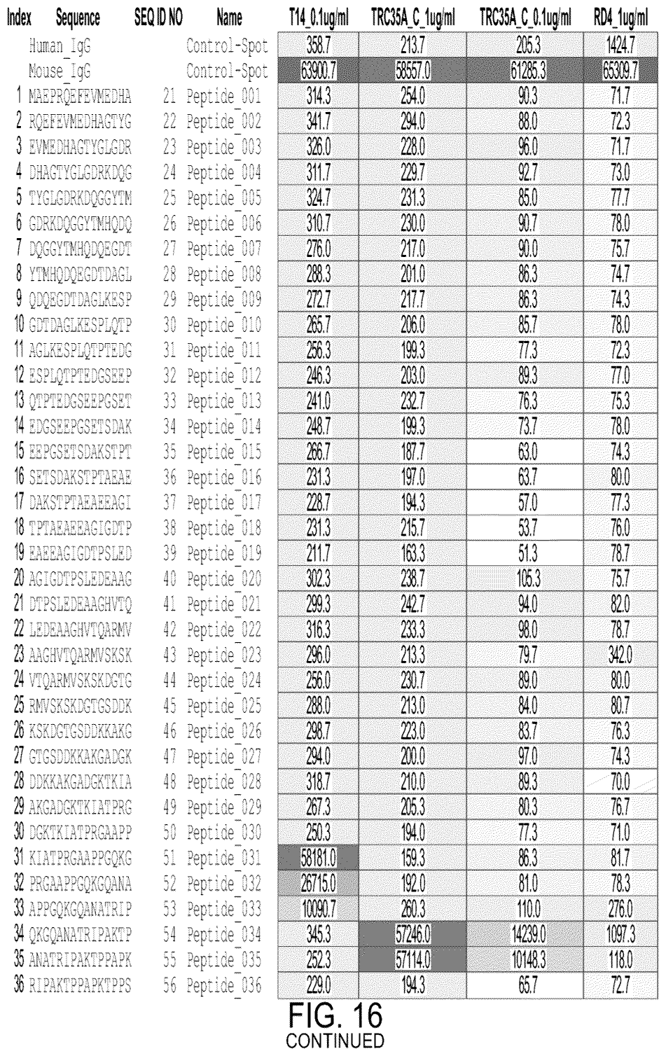

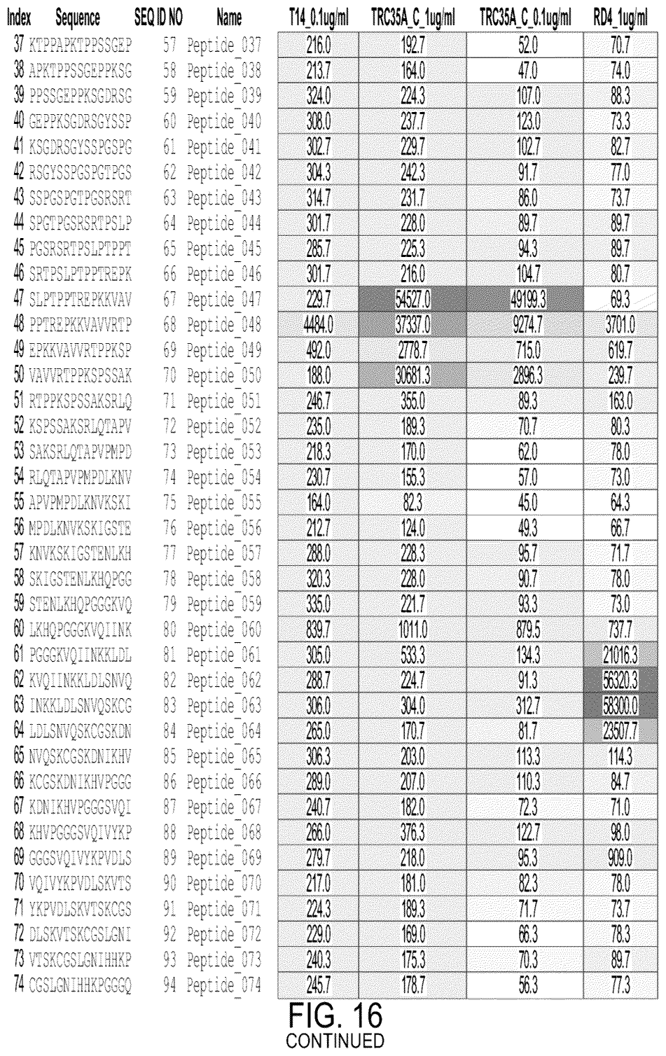

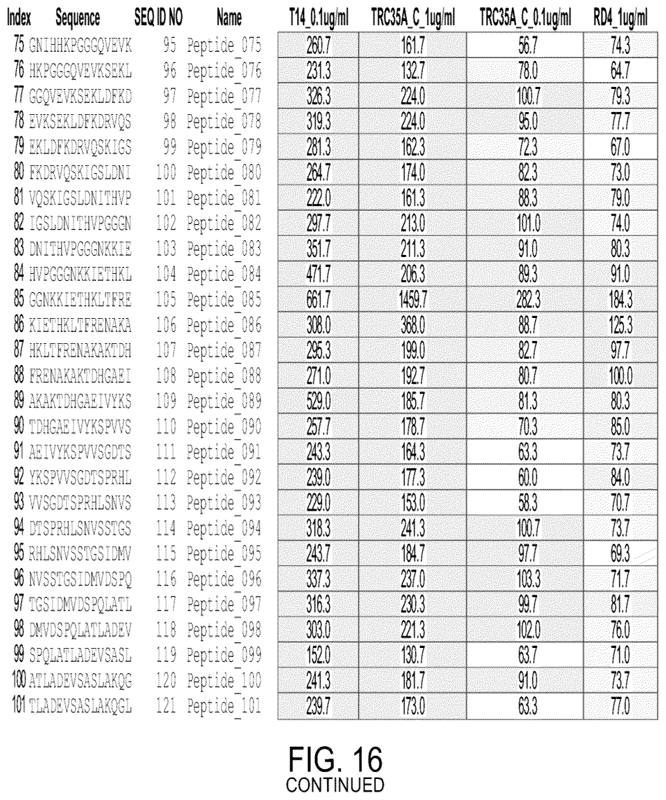

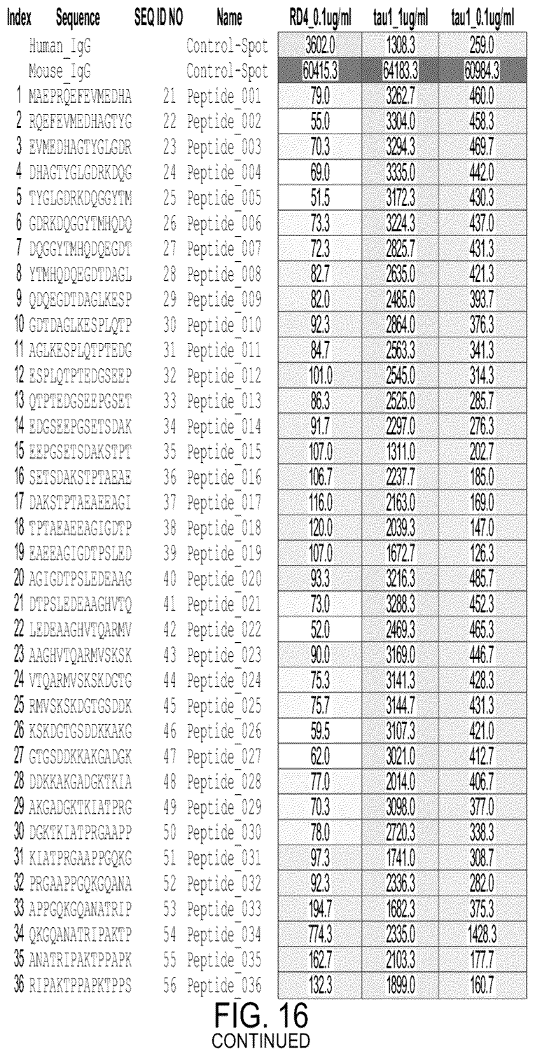

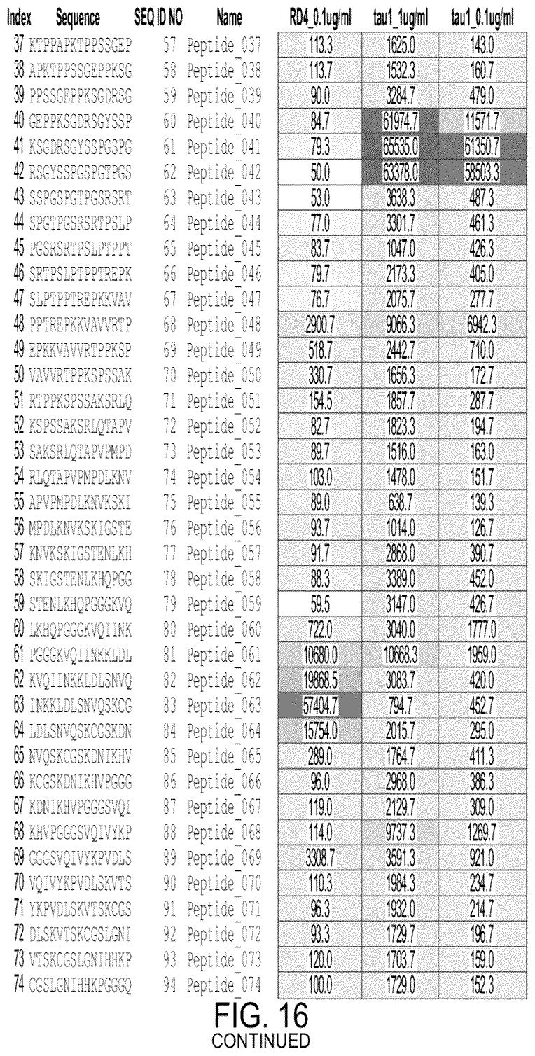

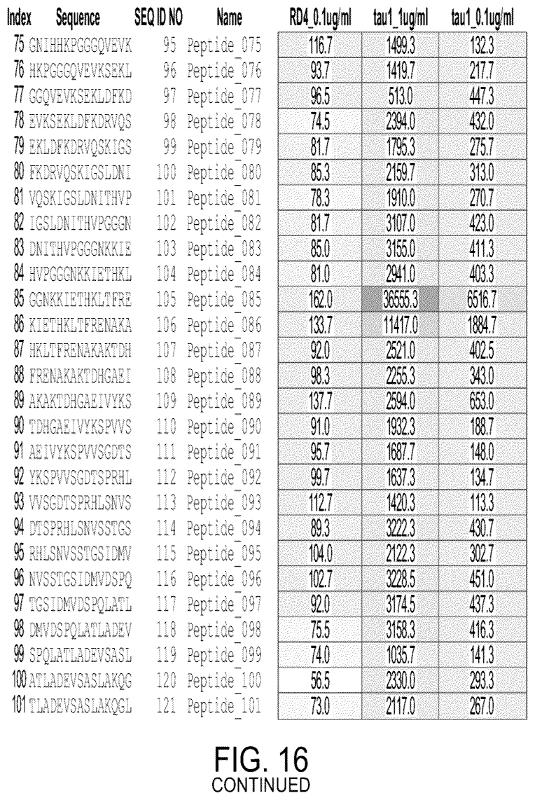

Disclosed herein are antibodies directed against aggregated tau/RNA complexes, and polypeptides. In some aspects, the antibody can bind an epitope having an amino acid sequence disclosed in FIG. 16. In some aspects, the epitope can be ANATRIPAKTP (SEQ ID NO: 122; amino acids 166-176 of SEQ ID NO: 20) or VAVV (SEQ ID NO: 123; corresponding to amino acids 226-229 of SEQ ID NO: 20).

In some aspects, the antibodies disclosed herein can include full-length antibodies, antibody fragments, single chain antibodies, bispecific antibodies, minibodies, domain antibodies, synthetic antibodies and antibody fusions, and fragments thereof.

As used herein, the term “antigen” is a molecule capable of being bound by an antibody or T-cell receptor. In some aspects, binding moieties other than antibodies can be engineered to specifically bind to an antigen, e.g., aptamers, avimers, and the like.

The term “antibody” or “immunoglobulin” is used to include intact antibodies and binding fragments/segments thereof. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Typically, fragments compete with the intact antibody from which they were derived for specific binding to an antigen. Fragments include separate heavy chains, light chains, Fab, Fab′ F(ab′)₂, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The term “antibody” can include five different classes of human immunoglobulins, namely IgG, IgA, IgM, IgD, and IgE. In some aspects, the disclosed antibodies can be an IgG class of antibody which can be classified into the 4 subclasses of IgG1, IgG2, IgG3, and IgG4. In some aspects, the disclosed antibodies can be an IgA class of antibody which, can be classified into the 2 subclasses of IgA1 and IgA2. The basic structure of immunoglobulin is made up of 2 homologous L chains (light chains) and 2 homologous H chains (heavy chains). The immunoglobulin class and subclass are determined by H chains. In some aspects, the antibody or antibodies or variants or fragments thereof can be an IgG4.

While antigen-binding specificity is maintained, antibody stability of IgG4 can be improved. In some aspects, the antibody can be improved, for example, by substituting arginine (R) of IgG4 with glutamic acid (E), phenylalanine (F), isoleucine (I), asparagine (N), glutamine (Q), serine(S), valine (V), tryptophan (W), tyrosine (Y), lysine (K), threonine (T), methionine (M), or leucine (L).

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. In some aspects, the at least one amino acid change or substitution can decrease deamidation. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 6-11, 14, 15, or 17-19. In some aspects, the variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 6-11, 14, 15, or 17-19. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, (5) reduces or decreases deamidation; and (6) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

In some aspects, the at least one amino acid change can be to substitute an NG motif (amino acid asparagine followed by a glycine). In some aspects, the glycine residue can be substituted or replaced with hydrophobic amino acid residue. In some aspects, the glycine residue can be substituted or replaced with alanine, aspartic acid, glutamic acid, or valine. In some aspects, the glycine residue can be substituted or replaced with arginine, lysine, or glutamine.

Many methods have been developed for chemical labeling and enhancement of the properties of antibodies and their common fragments, including the Fab and F(ab′)₂ fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)₂ fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g., in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “antigen-binding portion” of an antibody (or simply “antibody portion”), as used herein, refers to one or more fragments of an antibody that retain the ability to specifically bind to an antigen (e.g., tau/RNA complex or aggregated tau/RNA complex). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL/VK, VH, CL and CH1 domains; (ii) a F(ab′)₂ fragment, a bivalent fragment comprising two Fab fragments linked by a disulfide bridge at the hinge region; (iii) a Fab′ fragment, which can be an Fab with part of the hinge region (see, Fundamental Immunology (Paul ed., 3rd ed. 1993); (iv) a Fd fragment consisting of the VH and CH1 domains; (v) a Fv fragment consisting of the VL and VH domains of a single arm of an antibody; (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10⁻¹⁰ M⁻¹ to about 10¹¹ M⁻¹. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the antibodies described herein can be specifically bind to their intended target. In some aspects, the antibodies described herein have no off site binding. For example, the antibodies described herein do not bind or are not distributed to the heart, liver or spinal cord.

The antibodies described herein can be variants including, without limitation, a fragment (e.g., an Fab fragment or an F(ab′)₂ fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the Chi domain of the heavy chain. In contrast, digestion with pepsin yields the F(ab′)₂ fragment that has two antigen-binding sites and is still capable of cross-linking antigen.

Fab′ fragments differ from Fab fragments in that they include additional residues at the C-terminus of the Chi domain, including one or more cysteine residues from the antibody hinge region. The cysteine residues of the constant domains bear a free thiol group. F(ab′)₂ antibody fragments are pairs of Fab′ fragments linked by cysteine residues in the hinge region. Other chemical couplings of antibody fragments are also known in the art.

The Fv region is a minimal fragment that contains a complete antigen-recognition and binding site consisting of one heavy chain and one light chain variable domain. The three CDRs of each variable domain interact to define an antigen-biding site on the surface of the VH-VL dimer. Collectively, the six CDRs confer antigen-binding specificity to the antibody. As would be known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the VH and VL domains that allows the scFv to form a desired three-dimensional structure for antigen binding (see, e.g., Pluckthun, The Pharmacology of Monoclonal Antibodies, Rosenburg and Moore Eds., Springer-Verlag, New York, 113:269-315. 1994).

In some aspects, the antibody can be a diabody. Diabodies are small antibody fragments that have two antigen-binding sites. Each fragment contains a VH domain concatenated to a VL domain. However, since the linker between the domains is too short to allow pairing between them on the same chain, the linked Vh-Vl domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In some aspects, an antibody or a fragment thereof that binds to at least a portion of an aggregated tau/RNA complex and can treat or prevent a neurodegenerative disease or disorder, a tauopathy, dementia or one or more symptoms associated therewith are contemplated. In some aspects, the TRC35 antibody can be a monoclonal antibody, polyclonal antibody or a humanized antibody. Thus, by known means and as described herein, polyclonal or monoclonal antibodies, antibody fragments, and binding domains and CDRs (including engineered forms of any of the foregoing) may be created that are specific to an aggregated tau/RNA complex, one or more of its respective epitopes, or conjugates of any of the foregoing, whether such antigens or epitopes are isolated from natural sources or are synthetic derivatives or variants of the natural compounds.

Examples of antibody fragments suitable include without limitation: (i) the Fab fragment, consisting of VL, VH, CL, and CH1 domains; (ii) the “Fd” fragment consisting of the VII and Cm domains; (iii) the “Fv” fragment consisting of the VL and VH domains of a single antibody; (iv) the “dAb” fragment, which consists of a VH domain; (v) isolated CDR regions; (vi) F(ab′)₂ fragments, a bivalent fragment comprising two linked Fab fragments; (vii) single chain Fv molecules (“scFv”), wherein a VII domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made (Hu et al., 1996).

Antibody-like binding peptidomimetics are also contemplated. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a tau/RNA complex, in order to produce antibodies specific for tau/RNA complexes. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes.

A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because the antibody producing cells are derived from a single B-lymphocyte cell line. The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a tau/RNA complex antigen with an immortal cell (. This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepared rabbit peripheral blood mononuclear cells of immunized rabbits and further selected for tau/RNA complex binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. Tau/RNA complex specific binding Fab may be selected out through multiple rounds enrichment panning and sequenced. Selected tau/RNA complex binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In some aspects, the antibody can be a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that antibodies to tau/RNA complexes will have the ability to neutralize or counteract the effects of tau/RNA complexes regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described herein, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds aggregated tau/RNA complexes.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Described herein are antibodies and antibody-like molecules against aggregated tau/RNA complexes, polypeptides and peptides that are linked to at least one agent to form an antibody conjugate or payload. In order to increase the efficacy of antibody molecules as diagnostic or therapeutic agents, to the antibody can be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In some aspects, the anti-tau/RNA complex antibody (e.g., TRC35 antibody or TRC1 antibody) described herein can comprise a heavy chain immunoglobulin variable region comprising the sequence disclosed in Table 2. In some aspects, the anti-tau/RNA complex antibody (e.g., TRC35 antibody or TRC1 antibody) described herein can comprise a heavy chain immunoglobulin variable region comprising SEQ ID NO: 1 or SEQ ID NO: 12.

In some aspects, the anti-tau/RNA complex antibody (e.g., TRC35 antibody or TRC antibody) described herein can comprise a light chain immunoglobulin variable region comprising the sequence disclosed in Table 2. In some aspects, the anti-tau/RNA complex antibody (e.g., TRC35 antibody or TRC1 antibody) described herein can comprise a light chain immunoglobulin variable region comprising SEQ ID NO: 2 or SEQ ID NO: 13.

Disclosed herein are antibodies or fragments thereof that bind to human aggregated tau/RNA complexes. In some aspects, the antibody or fragment thereof comprises a variable light chain comprising a sequence having at least 90% identity to a variable light chain amino acid sequence provided in Table 2. In some aspects, the antibody or fragment thereof comprises a variable light chain comprising a sequence having at least 90% identity to SEQ ID NO: 2 or SEQ ID NO: 13.

Disclosed herein are antibodies or fragments thereof that bind to human aggregated tau/RNA complexes. In some aspects, the antibody or fragment thereof comprises a variable heavy chain comprising a sequence having at least 90% identity to a variable heavy chain amino acid sequence provided in Table 2. In some aspects, the antibody or fragment thereof comprises a variable heavy chain comprising a sequence having at least 90% identity to SEQ ID NO: 1 or SEQ ID NO: 12.

Disclosed herein are antibodies or fragments thereof that bind to human aggregated tau/RNA complexes. In some aspects, the antibody or fragment thereof comprises a variable heavy chain comprising a sequence having at least 90% identity to a sequence set forth in Table 1, and a variable light chain comprising a sequence having at least 90% identity to a sequence set forth in Table 2. In some aspects, the antibody or fragment thereof comprises a variable heavy chain comprising a sequence having at least 90% identity to SEQ ID NO: 1, and a variable light chain comprising a sequence having at least 90% identity to SEQ ID NO: 2. In some aspects, the antibody or fragment thereof comprises a variable heavy chain comprising a sequence having at least 90% identity to SEQ ID NO: 12, and a variable light chain comprising a sequence having at least 90% identity to SEQ ID NO: 13.

In some instances, the disclosed antibodies or fragments thereof can be bispecific. For example, the antibody or fragment thereof can comprise a first Fab region comprising the heavy and light chain as disclosed in Table 2 and a second Fab region comprising the heavy and light chain of any of the sequences disclosed in Table 2, wherein the first and second Fab regions can be different. In some aspects, the antibody or fragment thereof can comprise a first Fab region comprising the heavy chain sequence of SEQ ID NO: 1 and light chain second of SEQ ID NO: 2 and a second Fab region comprising the heavy chain sequence of SEQ ID NO: 1 and light chain of SEQ ID NO: 2, wherein the first and second Fab regions can be different. In some aspects, the antibody or fragment thereof can comprise a first Fab region comprising the heavy chain sequence of SEQ ID NO: 12 and light chain second of SEQ ID NO: 13 and a second Fab region comprising the heavy chain sequence of SEQ ID NO: 12 and light chain of SEQ ID NO: 13, wherein the first and second Fab regions can be different.

In some instances, the bispecific antibodies can be trifunctional.

In some instances, the disclosed antibodies or fragments thereof can be mouse, human, humanized, chimeric, or a combination thereof.

In some instances, the disclosed antibodies or fragments thereof are monoclonal. METHODS

Disclosed herein are methods of treating a tauopathy, dementia, or ocular pharyngeal muscular dystrophy in a subject with antibodies that bind aggregated tau/RNA complexes.

Disclosed herein are methods of inhibiting microtubule polymerization in a subject with antibodies that aggregated tau/RNA complexes. Disclosed herein are methods of ameliorating one or more symptoms associated with a tauopathy or ocular pharyngeal muscular dystrophy with antibodies that bind aggregated tau/RNA complexes.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of inhibiting microtubule polymerization in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13. Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

In some aspects, the methods disclosed herein can comprise administering to the subject a therapeutically effective amount of an antibody or fragment thereof disclosed herein. In some aspects, the antibody or fragment thereof can comprise a variable heavy chain comprising a sequence having at least 90% identity to a sequence set forth in Table 2. In some aspects, the antibody or fragment thereof can comprise a variable heavy chain comprising a sequence having at least 90% identity to SEQ ID NO: 1. In some aspects, the antibody or fragment thereof can comprise a variable heavy chain comprising a sequence having at least 90% identity to SEQ ID NO: 12. In some aspects, the antibody or fragment thereof can comprise a variable light chain comprising a sequence having at least 90% identity to a sequence set forth in Table 2. In some aspects, the antibody or fragment thereof can comprise a variable light chain comprising a sequence having at least 90% identity to SEQ ID NO: 2. In some aspects, the antibody or fragment thereof can comprise a variable light chain comprising a sequence having at least 90% identity to SEQ ID NO: 13.

In some aspects, any of the methods disclosed herein can comprise administering to the subject an effective amount of an expression vector encoding the antibody or fragment thereof. In some aspects, the antibody or fragment thereof can be administered in a pharmaceutically acceptable composition. In some aspects, the pharmaceutical composition can be lyophilized. In some aspects, the antibody or fragment thereof can be administered systemically. In some aspects, the antibody or fragment thereof can be administered intravenously, intradermally, intratumorally, intramuscularly, intraperitoneally, subcutaneously, or locally. In some aspects, the antibody or fragment thereof can be a humanized antibody or humanized fragment thereof. In some aspects, the antibody can be an IgG, IgM, IgA, IgD, IgE, or a genetically modified IgG class antibody. In some aspects, the antibody can be an IgG class of antibody, wherein the IgG class antibody is an IgG1, IgG2, IgG3, or IgG4 class antibody.

In some aspects, any of the methods disclosed herein (e.g., a tauopathy or ocular pharyngeal muscular dystrophy) can further comprise administering at least a second therapeutic agent, a second therapy, or a combination thereof to the subject.

In some aspects, in any of the methods disclosed herein the antibody or fragment thereof can bind to aggregated tau/RNA complexes. In some aspects, in any of the methods disclosed herein the antibody or fragment thereof can inhibit microtubule polymerization.

In some aspects, the antibody or fragment thereof can further comprise a tag sequence.

In some aspects, the antibody or fragment thereof can be a Fab fragment an Fab′ fragment or an F(ab′)₂ fragment.

### Treatment of Diseases

Disclosed herein are antibodies (e.g., the TRC35 antibody, the TRC1 antibody) and biological fragments thereof that can be used to treat a tauopathy, dementia, or ocular pharyngeal muscular dystrophy in a subject in need thereof.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a tauopathy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13. Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a dementia in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 2.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 1; and wherein the antibody or fragment thereof comprises a variable light chain comprising a sequence having at least 90% identity to SEQ ID NO: 2.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable light chain comprising a sequence having at least 90% identity to SEQ ID NO: 13.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a variable heavy chain comprising a sequence having at least 90% identity to SEQ ID NO: 12; and wherein the antibody or fragment thereof comprises a variable light chain comprising a sequence having at least 90% identity to SEQ ID NO: 13.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 9; a complementarity determining region light chain 2 (CDRL2) amino acid sequence of SEQ ID NO: 10; and a complementarity determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

Disclosed herein are methods of treating a ocular pharyngeal muscular dystrophy in a subject, the methods comprising administering to the subject a therapeutically effective amount of an antibody or a fragment thereof that can bind to aggregated tau/RNA complexes, wherein the antibody or fragment thereof comprises a light chain variable region and a heavy chain variable region, wherein the light chain variable region comprises a complementarity determining region light chain 1 (CDRL1) amino acid sequence of SEQ ID NO: 17; a complementarity determining region light chain 2 (CDRL2) amino acid sequence of SEQ ID NO: 18; and a complementarity determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with, diagnosed or at risk for a tauopathy, dementia, or ocular pharyngeal muscular dystrophy in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease or condition is delayed, hindered, or prevented, or the disease or condition or a symptom of the disease or condition is ameliorated or its frequency can be reduced. One or more of the symptoms can be less severe. Recovery can be accelerated in an individual who has been treated. In some aspects, the antibodies described herein can improve the quality of life of a subject with or at risk for a tauopathy, dementia, or ocular pharyngeal muscular dystrophy. In some aspects, the antibodies described herein can prevent one or more symptoms of a tauopathy, dementia, or ocular pharyngeal muscular dystrophy.

In some aspects, the methods can comprise administering an effective amount of the antibody to the subject. In some aspects, the method can comprise administering an effective amount of an expression vector encoding the antibody to the subject.

In some aspects, the subject has been diagnosed with Alzheimer's disease, a tauopathy, dementia, or ocular pharyngeal muscular dystrophy prior to the administering step.

In some aspects, a tauopathy can be a disorder with primary insoluble tau deposits. In some aspects, a tauopathy can be Alzheimer's disease, Pick disease, progressive supranuclear palsy, corticobasal degeneration, chronic traumatic encephalopathy, and globular glial tauopathy.

The compositions described herein can be formulated to include a therapeutically effective amount of the antibodies disclosed herein. In some aspects, antibodies disclosed herein can be contained within a pharmaceutical formulation. In some aspects, the pharmaceutical formulation can be a unit dosage formulation.

The therapeutically effective amount or dosage of any of the antibodies used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the disease or disease symptoms), the age and physical characteristics of the subject and other considerations known to those of ordinary skill in the art. Dosages can be established using clinical approaches known to one of ordinary skill in the art. A therapeutically effective dosage of an antibody disclosed herein can result in a decrease in severity of one or more disease symptoms, an increase in frequency and duration of disease symptom-free periods, or a prevention of impairment or disability due to the disease affliction. A therapeutically effective amount of a therapeutic compound or antibody can decrease tumor metastasis, or otherwise ameliorate symptoms in a subject.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the antibodies or compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The antibodies or compositions described herein can be administered in conjunction with other therapeutic modalities to a subject in need of therapy. The present compounds can be given to prior to, simultaneously with or after treatment with other agents or regimes. For example, the antibodies disclosed herein can be administered alone or in conjunction with standard therapies used to treat tauopathies or ocular pharyngeal muscular dystrophy. In some aspects, any of the antibodies or compositions described herein can be administered or used together with a second therapy.

### Pharmaceutical Compositions

Disclosed herein are compositions, e.g., pharmaceutical compositions, comprising one or a combination of monoclonal antibodies, or antigen-binding portion(s) thereof formulated with a pharmaceutically acceptable carrier. Such compositions may include one or a combination of (e.g., two or more different) antibodies, or immunoconjugates described herein. For example, a pharmaceutical composition of the invention can comprise a combination of antibodies that bind to different epitopes on the target antigen or that have complementary activities.

Pharmaceutical compositions of the invention also can be administered as combination therapy, i.e., combined with other agents. For example, the combination therapy can include an anti-aggregated tau/RNA complex (e.g., TRC35 antibody) antibody combined with at least one other therapeutic agent or therapy. In some aspects, the second therapeutic agent can be a bisphosphonate, calcitonin, teriparatide, denosumab or romosozumab. In some aspects, the bisphosphonate can be alendronate, ibandronate, resendronate, or zoledronic acid. In some aspects, the second therapy can be exercise.

As used herein, the phrase “pharmaceutically acceptable carrier” includes any solvents, dispersion media, coatings, antibacterial and antifungal agents, isotonic and absorption delaying agents, and the like that are physiologically compatible. Preferably, the carrier can be suitable for intravenous, intramuscular, subcutaneous, or parenteral administration (e.g., by injection or infusion). Depending on the route of administration, the active compound, i.e., antibody, or immunoconjugate, may be coated in a material to protect the compound from the action of acids and other natural conditions that may inactivate the compound.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated herein, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, 5 mg/kg to 10 mg/kg, 10 mg/kg to 15 mg/kg, 15 mg/kg to 20 mg/kg or 20 mg/kg to 25 mg/kg of the host body weight. In some aspects, the dosages can be 0.3 mg/kg body weight, 1 mg/kg body weight, 3 mg/kg body weight, 5 mg/kg body weight or 10 mg/kg body weight or within the range of 1-10 mg/kg. In some aspects, the dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight or 30 mg/kg body weight or within the range of 1-30 mg/kg. In some aspects, the dosages can be about 15, 16, 17, 18, 19, 20, 21, 22, 23, 24 or 25 mg/kg body weight. In some aspects, the dosages can be 5 mg/kg body weight. In some aspects, the dosages can be 15 mg/kg body weight. In some aspects, the dosages can be 20 mg/kg body weight. In some aspects, the dosages can be 25 mg/kg body weight. An exemplary treatment regime entails administration once per week, once every two weeks, once every three weeks, once every four weeks, once a month, once every 3 months or once every three to 6 months. Preferred dosage regimens for the antibodies of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection, and includes, without limitation, intravenous, intramuscular, intraarterial, intraperitoneal, transtracheal, subcutaneous, subcuticular, intraarticular injection and infusion.

In some aspects, the antibody disclosed herein can be administered systemically. In some aspects, the antibody disclosed herein can be administered intravenously, intradermally, intratumorally, intramuscularly, intraperitoneally, subcutaneously, or locally.

Combination Treatments. The compositions and methods described herein can involve an antibody or an antibody fragment thereof against tau/RNA complexes to, for example, treat one or more tauopathies, inhibit microtubule polymerization in combination with a second therapeutic or additional therapy.

The methods and compositions, including combination therapies, enhance the therapeutic or protective effect, and/or increase the therapeutic effect of another therapeutic or therapy. Therapeutic and prophylactic methods and compositions can be provided in a combined amount effective to achieve the desired effect. This process may involve contacting the cells with both an antibody or antibody fragment and a second therapy. A tissue or cell can be contacted with one or more compositions or pharmacological formulation(s) comprising one or more of the agents, or by contacting the tissue and/or cell with two or more distinct compositions or formulations, wherein one composition provides 1) an antibody or antibody fragment, 2) a second therapy, or 3) both an antibody or antibody fragment and a second therapy.

The terms “contacted” and “exposed,” when applied to a cell, are used herein to describe the process by which a therapeutic agent is delivered to a target cell or are placed in direct juxtaposition with the target cell.

The antibodies and biological fragments thereof can be administered before, during, after, or in various combinations relative to any second treatment or therapy. The administrations may be in intervals ranging from concurrently to minutes to days to weeks. In aspects where the antibody or antibody fragment is provided to a patient separately from a second treatment or therapy, one would generally ensure that a significant period of time did not expire between the time of each delivery, such that the two compounds would still be able to exert an advantageously combined effect on the patient. In such instances, it is contemplated that one may provide a patient with the antibody therapy and the a second treatment or therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no second treatment or therapy is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and a second therapy is “B”:

A/B/A B/A/B B/B/A A/A/B A/B/B B/A/A A/B/B/B B/A/B/B B/B/B/A
B/B/A/B A/A/B/B A/B/A/B A/B/B/A B/B/A/A B/A/B/A B/A/A/B
A/A/A/B B/A/A/A A/B/A/A A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

### Kits and Diagnostics

Disclosed herein are kits comprising one or more of the disclosed antibodies and/or other therapeutic and delivery agents. In some aspects, the kit can be used for preparing and/or administering a therapy disclosed herein. The kit may comprise one or more sealed vials containing any of the pharmaceutical compositions disclosed herein. The kit may include, for example, at least one antibody or fragment thereof disclosed herein as well as reagents to prepare, formulate, and/or administer the components one or more of the compositions disclosed herein or perform one or more steps of the inventive methods. In some aspects, the kit may also comprise a suitable container, which can be a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Tau-RNA Complexes Inhibit Microtubule Polymerization and Drive Disease-Relevant Conformation Change

While the rapid progression of tauopathy research has answered many questions about tau aggregation, seeding, and pathological spread, a fundamental understanding of the initial triggers of pathological tau aggregation in vivo is lacking. Here, tau's interaction with RNA, a polyanion known to potently seed tau aggregation (Kampers T, et al. FEBS Letters. Dec. 16, 1996 1996; 399 (3): 344-349; and Zhang X, et al. RNA stores tau reversibly in complex coacervates. PLOS Biol. July 2017; 15 (7): e2002183), was further investigated. A series of reagents and assays were developed to detect and measure tau activity as an RNA binding protein and to address the impact of tau/RNA binding activity on tau normal function and tau neuropathology.

Materials and methods. Preparation of recombinant tau. Recombinant 6×His tagged human tau protein (isoforms 1N4R and 1N3R) were expressed in E. coli and purified using a boiling lysis preparation and native nickel resin chromatography (Baker J D, et al. PLOS Biol. June 2017; 15 (6): e2001336). Recombinant untagged human tau (isoform 1N4R) was expressed in E. coli and purified using a boiling lysis preparation (Hong M, et al. Science. 1998 1998; 282:1914-1917; and Barghorn S, et al. Biochemistry. Feb. 17 2004; 43 (6): 1694-703).

Microtubule Assembly Assays. Microtubule assembly assays were performed in a low 30 μl reaction volume format in flat bottom 384 well plates (Kiris E, et al. J Biol Chem. Apr. 22 2011; 286 (16): 14257-70). Porcine tubulin (20 μM final concentration, #T240, Cytoskeleton Inc., Denver, CO) was mixed with 2 μM recombinant 1N4R tau and varying concentrations of Poly(A) RNA (P9403, Sigma Chemical, St. Louis, MO) in BRB80 [80 mM PIPES, 1 mM EGTA, 1 mM MgSO₄, pH 6.8], 2 mM GTP, and 1 mM DTT. Reactions were assembled on ice and Poly(A), GTP, and DTT were diluted to appropriate working stock concentrations in BRB80 prior to reaction assembly. Tubulin with 20 μM taxol was utilized as a positive control for assay conditions and tubulin quality. Once the reaction components were added, the plate was allowed to incubate at room temperature for 2 minutes, the bottom wiped with a Kimwipe, and placed in a Perkin Elmer Envision plate reader maintained at 37° C. Absorbance readings (340 nm) were taken every 60 seconds for three hours. Absorbance read data was normalized by subtracting the mean of the first 5 reads from each absorbance read for each condition.

Analysis of tau/RNA interactions. Biotinylated RNAs were custom synthesized by Integrated DNA technologies (IDT, Coralville, IA). Tau/RNA interactions were analyzed using surface plasmon resonance (Ametek Reichert Technologies, Depew, NY) as recommended by the manufacturer for RNA/protein interactions using H2T as SPR running buffer [50 mM HEPES, 25 mM NaCl, 0.1% Tween-20, pH7.4] and a streptavidin SPR chip (catalog #13206071, Reichert SPR).

Generation of tau/RNA complexes. Recombinant untagged human tau was incubated with poly(A) RNA in buffer H2 for 16 hrs at 20° C. with shaking at 400 RPM.

Analysis of tau/RNA complexes by size exclusion chromatography. To evaluate tau aggregation or complex formation, equivalent mass of tau protein in the form of uncomplexed purified tau and tau/RNA complexes were analyzed using size exclusion chromatography using a GE AKTA purifier running a SUPERDEX 200 increase column at 1 ml/min (Dujardin S, et al. Nat Med. August 2020; 26 (8): 1256-1263) with H2 [50 mM HEPES, 25 mM NaCl, pH7.4] as the chromatography running buffer. Fractions were immunoblotted for tau using the Simple Western capillary-electrophoresis system, Peggy Sue (ProteinSimple, San Jose, CA), and following manufacturer's recommendations. Briefly, fractions were diluted with 5× Fluorescent Master Mix, boiled for 5 min at 95° C., cooled to 4° C. and loaded into a 384-well plate (12-230 kDa Size Separation module, catalog #SM-S001, ProteinSimple). Primary antibody to tau (1:10,000, catalog #A0024, Dako, Agilent Technologies) and goat anti-rabbit HRP (1:100, Jackson ImmunoResearch) were diluted in Antibody Diluent 2 (No Secondary Detection Module, catalog #DM-003, ProteinSimple). Samples were run and analyzed with the default assay in Compass for SW Version 4.0.0 (ProteinSimple).

C. elegans husbandry and generation of poly(A) transgenic C. elegans. C. elegans strains were maintained (Brenner S. Genetics. 1974 1974; 77:71-94) at 20° C. The canonical wild-type C. elegans strain, Bristol N2, was used as the genetic background for transgenesis and as the wild-type C. elegans control strain. A transgene encoding poly(A)₄₅ RNA was driven by the C. elegans U6 RNA polymerase III promoter and universal TTTT RNA polymerase III terminate (Taylor L M, et al. Mol Neurodegener. Feb. 6 2018; 13 (1): 7). The transgene was injected into N2 at a concentration of 100 ng/μL along with Pmyo-3::mCherry (20 ng/μL) as a co-injection marker to produce worms carrying extra-chromosomal arrays. These generated lines were irradiated with UV to integrate the extra-chromosomal array into the genome (Mariol M C, et al. J Vis Exp. Dec. 9 2013; (82): e50773). Successfully integrated lines were identified by isolating individual worms with 100% transmission of the Pmyo-3::mCherry marker and outcrossed with N₂ males three times. The poly(A)₄₅ expressing strain described is CK2362. The tau transgenic strain used, CK144, expresses wild type 1N4R tau from the aex-3 promoter (Taylor L M, et al. Mol Neurodegener. Feb. 6 2018; 13 (1): 7). Bigenic animals expressing both poly(A)₄₅ and wild type tau are CK2408 and were generated by crossing CK144 with CK2362 and assessed for the impact of poly(A) on tauopathy related phenotypes (Kow R L, et al. Loss of aly/ALYREF suppresses toxicity in both tau and TDP-43 models of neurodegeneration. Geroscience. Feb. 4 2022).

C. elegans immunoblotting. Staged populations of C. elegans for the above described strains were grown to day 1 of adulthood and harvested (Kow R L, et al. Loss of aly/ALYREF suppresses toxicity in both tau and TDP-43 models of neurodegeneration. Geroscience. Feb. 4 2022). Frozen samples were resuspended in 5× Sample Buffer (5% SDS, 200 mM DTT, 50 mM Tris pH 6.8, 5 mM EDTA, 50% sucrose, 0.05% Bromophenol Blue), with volume equivalent to four times the pellet weight (4 μL/mg of packed animals). Samples were sonicated on ice for 3 bursts of 15 seconds at 70% power. Sonicated lysates were denatured for 10 minutes in a 95° C. heat block then cooled and briefly centrifuged. For each sample, 10 μL of supernatant was loaded and run on a 4-15% Tris-HCl SDS Page gel with appropriate protein standards. The proteins were transferred to PVDF in Tris-Glycine-methanol transfer buffer as per manufacturer recommendations (Bio-Rad). PVDF membrane was blocked in 5% w/v dry milk in PBS solution for an hour then treated overnight at 4° C. in primary antibody for total tau (1:500,000, catalog A0024, Dako, Agilent Technologies), pTau pS202 (CP13, 1:1,000), or pTau pS396/404 tau (PHF1, 1:1,000). Tubulin (Developmental Studies Hybridoma Bank (DSHB) anti-β Tubulin E7-s) was used as a load control at 1:5,000. The following day, primary antibody was removed, and the membrane washed three times in 1×PBS+1% Tween20 for 10 minutes each wash. Secondary antibody was then applied in 5% w/v dry milk in PBS solution and allowed to blot for 2 hours, followed by an additional three 1×PBS+1% Tween20 washes. Immunoreactivity was visualized using ECL (BioRad Clarity Western ECL Substrate 170-5061) with a LI-COR Odyssey Fc system (LI-COR Biosciences, Lincon, NE) and quantified using LI-COR Image Studio Software.

Immunization of mice with tau/RNA complexes. Tau RNA complexes as described herein were used to immunize mice to produce monoclonal antibodies using standard operating procedures (Elnaggar M M, et al. Vet Immunol Immunopathol. October 2017; 192:54-59). To produce tau antibodies efficiently by limiting self-antigen recognition, tau knockout mice were used for immunization (Dawson H N, et al. J Cell Sci. 2001 2001; 114:1179-1187). Briefly, tau/RNA complexes were mixed in Sigma Adjuvant System at 50% (w/v) as an emulsion prior to i.p. immunization as recommended by the manufacturer (Sigma Chemical, catalog S6322). Mice were immunized with 200 μg of tau/RNA complex and received an identical boost immunization 3 weeks after initial immunization. Spleens were harvested 3 days after boosting for hybridoma production. Clonal hybridoma lines were screened based on TRC binding activity and the TRC35 expressing hybridoma monoclonal line was isolated and cloned. TRC35 monoclonal hybridoma lysate was isotyped using Thermo Rapid Elisa mAb isotyping reagents (Thermo catalog 37503).

Brain Specimens. Samples of postmortem brain tissue were obtained from research participants in the University of Washington (UW) Alzheimer's Disease Research Center and the Kaiser Permanente Washington Health Research Institute Adult Changes in Thought (ACT) Study via the UW BioRepository and Integrated Neuropathology (BRaIN) Laboratory. Alzheimer's disease brain donors (n=19) were chosen based upon the clinical diagnosis of dementia and neuropathologically-confirmed Alzheimer's disease neuropathologic change (ADNC) sufficient to explain dementia (intermediate or high). Brain tissues used as controls for this study (n=7) were derived from age-matched cognitively normal research participants with neuropathologically-confirmed not-low levels of ADNC. Fixation of donor brains occurred by immersion in 10% neutral buffered formalin for at least two weeks, followed by coronal slicing and sampling that included dorsolateral prefrontal cortex, hippocampus, amygdala and cerebellum, which were processed and embedded in paraffin, and sectioned at 5-micron thickness according to routine protocols for neuropathological analysis as described herein.

Purification of fibrillar insoluble tau from Alzheimer's disease postmortem brain. Detergent-insoluble fibrillar tau protein was purified from Alzheimer's disease brain donors with neuropathologically confirmed high pathological tau burden (Braak stage VI). Temporal lobe cortex brain specimens frozen at autopsy from Alzheimer's disease donors were homogenized, repeatedly extracted with excess 1% sarkosyl until insoluble tau fibrils were highly enriched and soluble tau was depleted, with detergent-insoluble tau representing the major protein species present in these Alzheimer's disease tau extracts (Guo J L, et al. J Exp Med. Nov. 14 2016; 213 (12): 2635-2654).

Characterization of TRC35 monoclonal antibodies. TRC35 monoclonal antibodies were used to dot blot equivalent amounts (20 ng) E. coli-expressed native recombinant tau protein and Alzheimer's disease tau (ADtau) fibrillar material. ADtau was standardized by quantitative immunoblot against purified recombinant tau with pan-tau anti-tau antibody (catalog A0024, Dako, Agilent Technologies) such that 20 ng of E. coli-expressed native recombinant tau protein and ADtau fibrillar material exhibited equivalent immunoreactivity. Protein preparations were not denatured prior to blotting to preserve native conformations expected of a conformation dependent monoclonal antibody. Dot blots were performed using the Bio-Dot Microfiltration Apparatus as recommended by the manufacturer (Bio-Rad, catalog #170-6545). Membranes were blocked in blocking solution [20 mM Tris, 500 mM NaCl, 1% bovine serum albumin (w/v) | and probed with TRC35 at 1:50,000 as primary antibody in TTBS buffer [20 mM Tris, 500 mM NaCl, 1% bovine serum albumin (w/v), 0.5% (w/v) Tween20]. Blots were washed 3 times in TTBS to remove excess primary antibody. Goat anti rabbit IgG secondary antibodies were used at 1:10,000 in TTBS. Blots were washed 3 times in TTBS to remove excess secondary antibody. Immunoreactivity was measured using Clarity Western ECL reagents (Bio-Rad) Images were scanned and quantitated using LI-COR Image Studio software. Epitope profile experiments were conducted as a fee for service project at JPT Peptide Technologies, Inc (Berlin Germany). Epitope profiling employed an array of 101 overlapping peptides tiling over the entire coding sequence of tau protein. The profiling experiment was performed with antibodies diluted to 1 μg/ml and 0.1 μg/ml with TBS blocking buffer (Pierce). Subsequent to sample incubation, secondary fluorescently labeled anti-mouse-IgG at 1 μg/ml was added into the corresponding wells and left to react for 1 hour. After washing and drying, the slide was scanned with a high-resolution laser scanner at 635 nm to obtain fluorescence intensity profiles.

Transgenic Animal modelling. C57B6/J was used as the control strain of mice and the tau transgenes were maintained in a congenic state on this background. For the mouse analyses, both sexes were used and no animals were excluded. The PS19 tau transgenic mouse model expressing human P301S mutant human tau was used in this study. This mouse model is well-characterized and has a highly progressive tauopathy related phenotype. In addition, a milder mouse model of tauopathy driven by wild type human tau expression (Tau4RTg2652) was also examined. This mouse line exhibits early-stage tau pathology, including phosphorylated tau, but not neurofibrillary degeneration (Yoshiyama Y, et al. Neuron. Feb. 1 2007; 53 (3): 337-51). PS19 mice (n=12 at 9 months, n=4 at 3 months), Tau4RTg2652 mice (n=7 at 3 months), and WT mice (n=6 at 3 months of age) were anesthetized and fixed by transcardial perfusion with 4% paraformaldehyde. Brains were removed and paraffin embedded for sectioning. Coronal sections (9 microns) were prepared and stored at 4° C. until use.

Neuropathological Evaluation. Human and mouse brain sections were deparaffinized, rehydrated through alcohols, and processed through antigen retrieval steps consisting of heat pretreatment in citrate buffer by either microwave or autoclave per antibody-specific protocols. Sections were treated for endogenous peroxidases with 3% hydrogen peroxide in PBS (pH 7.4), blocked in 5% non-fat milk in PBS, and incubated with primary antibodies overnight at 4° C. Biotinylated secondary goat anti-mouse or goat anti-rabbit antibody was applied for 45 min at room temperature. Finally, sections were incubated in an avidin-biotin complex with streptavidin-HRP (Vector's Vectastain Elite ABC-HRP kit, Burlingame, CA) and the reaction product was visualized with 0.05% diaminobenzidine (DAB)/0.01% hydrogen peroxide in PBS. Negative controls consisted of full protocol except primary antibody. For double label immunofluorescence experiments, AlexaFluor 488 nm goat anti-mouse and AlexaFluor 594 nm goat anti-rabbit secondary antibodies (molecular Probes) were used and autofluorescence was quenched with 0.1% Sudan Black. Digital images were obtained using a Leica DM6 microscope with a DFC 7000 digital camera (Leica Microsystems, Wetzlar, Germany) and imported into Adobe Photoshop (Adobe Inc, San Jose, CA).

Quantitative analysis of immunohistochemistry. HALO digital image software (Indica Labs) was used to quantify TRC35 and AT180 immunoreactivity in mouse and human brain. Brain sections were manually annotated around the regions of interest, average staining intensity for each antibody was determined to allow quantification without contribution of background staining, and a common threshold was then applied to all sections for that assay. Data represent the area of positive immunoreactivity within the region of interest divided by the total annotated area. This value was then multiplied by the average optical density of immunoreactivity to yield the final normalized IR area×OD. Data are displayed as the mean+/−SEM. A two tailed Student's t-test was used to assess differences in immunoreactivity between experimental groups. Statistical analysis and graphing were performed using the Prism V8.3 software package (GraphPad).

Cell Culture. ReNcell VM cells (Millipore) were cultured in DMEM/F12 media (Sigma) supplemented with 2% B-27 (Thermofisher), 1% Glutamax (Thermofisher), 1% Penecillin/Streptomycin (1000 IU/ml), 10 units/ml heparin (Sigma), 50 ug/ml gentamicin (Thermofisher), 40 ng EGF (Sigma), and 40 ng bFGF (Sigma; (Chaudhuri A D, et al. J Biol Chem. May 8 2015; 290 (19): 12425-34; and Choi S H, et al. Nature. Nov. 13 2014; 515 (7526): 274-8)). Cells were grown on flasks pre-coated with laminin (Sigma) and split every 3-4 days. Cells were differentiated in the above media without growth factors for or more days, replacing media every 3-4 days.

Fluorescent Immunohistochemistry. ReNcell VM cells were cultured on 12 mm round coverslips coated with poly-D-lysine then laminin. Samples were fixed in a 4% formaldehyde solution, washed 3×5 min in PBS/Ca²⁺/Mg²⁺, then blocked in antibody buffer (PBS, 0.5% Triton X-100, 1 mM EDTA, 0.1% BSA, 0.05% NaN3) with 10% normal goat serum. Primary antibodies were applied and incubated overnight (TRC35 1:100; SP70 1:500, Invitrogen, catalog MA5-16404). Cells were washed 3×5 min in PBS/Ca²⁺/Mg²⁺, then re-blocked for 10 min. Appropriate Alexa dye-labeled secondary antibodies (1:1000, Invitrogen) were applied and incubated for 20 min at room temperature. Cells were again washed 3×5 min in PBS/Ca²⁺/Mg²⁺, counterstained with 300 nM DAPI, and mounted with ProLong Gold antifade (Molecular Probes). Microscopy was performed on a Delta Vision microscope (GE, Inc) using a 100× oil immersion objective, a sCMOS camera, and 2×2 binning. Image analysis was performed using softWoRx 6.0 Beta software (GE, Inc).

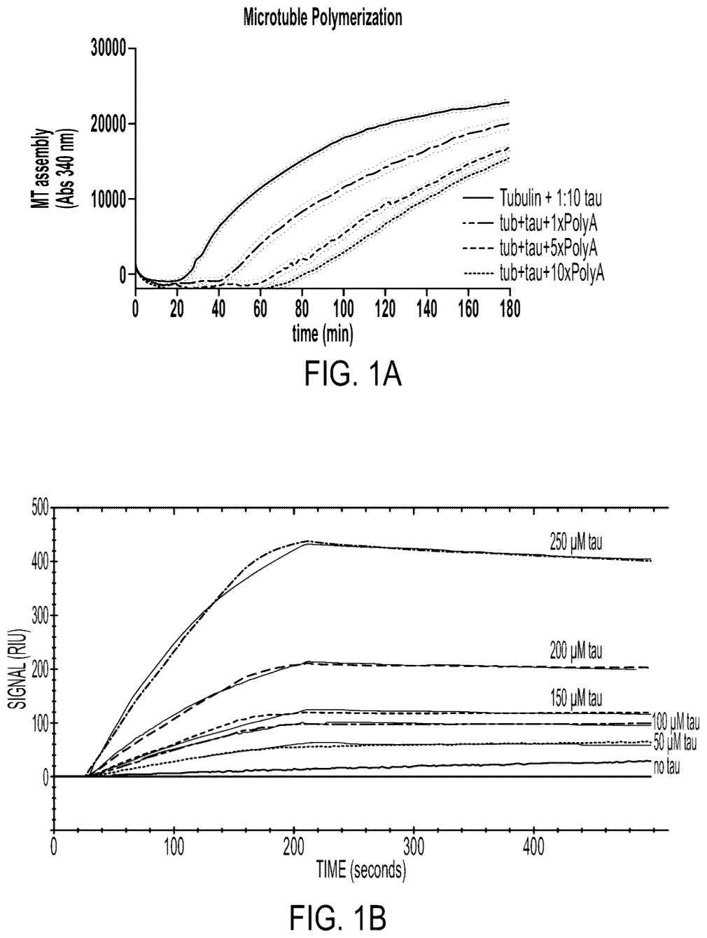

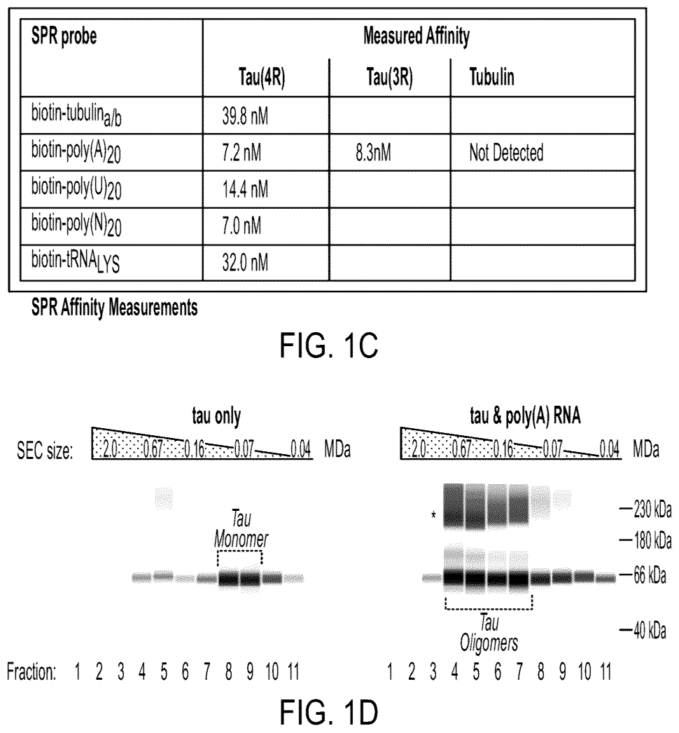

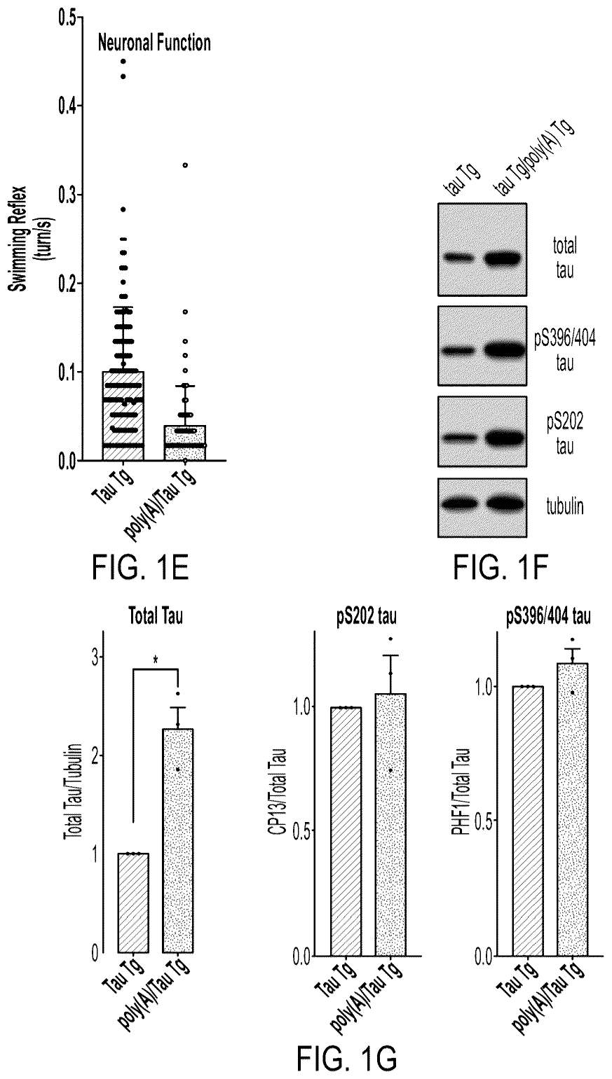

Results. Tau preferentially binds to RNA inhibiting microtubule assembly. Given that poly(A) RNA has been shown to block the canonical tau function of stimulating MT polymerization (Bryan J B, et al. Proc Natl Acad Sci USA. September 1975; 72 (9): 3570-4), it was examined whether RNA might compete with tau mediated microtubule assembly and found poly(A) RNA inhibits tau-mediated MT assembly in a dose dependent manner (FIG. 1A). Further, it was tested whether tau binds to RNA directly and competes with tubulin dimer binding. To measure tau's affinity for tubulin and different types of RNA molecules, a highly sensitive surface plasmon resonance (SPR) assay for RNA binding (Amano R and Sakamoto T. Methods Mol Biol. 2020; 2106:137-150) was used and adapted to tau. SPR has been suggested to be superior to electrophoretic mobility shift assay (EMSA) for characterization of RNA binding proteins (Matos R G, et al. Protein J. August 2010; 29 (6): 394-7). Recombinant human tau isoform 1N4R (the most abundant 4R brain isoform) was purified to measure binding to a series of biotinylated RNA probes and to observe high affinity interactions between tau and RNA (FIGS. 1B, 1C). It was observed that human tau prefers single stranded RNAs [poly(A), poly(U), poly(N) | over highly structured tRNA (IRNA_{LYS} K_{D}>30 nm, FIG. 1C). Control experiments also showed 3R tau binding poly(A) RNA with a similar affinity to 4R tau (8.3 vs 7.2 nM, respectively). However, tubulin does not detectably bind poly(A) RNA. The affinity of tau for tubulin was analyzed using a similar SPR assay with biotinylated a/B tubulin dimers in place of the RNA probe and observed that tau binds tubulin with high affinity (K_{D}=39.8 nM, FIG. 1C and Butner K A and Kirschner M W. J Cell Biol. November 1991; 115 (3): 717-30; and Kadavath H, et al. Proc Natl Acad Sci USA. Jun. 16 2015; 112 (24): 7501-6). Taken together, these data show that tau has a ˜5-fold greater affinity for linear homopolymeric RNA relative to tubulin.

Tau binding to RNA promotes formation of oligomeric tau complexes and exacerbates tauopathy phenotypes. It has been demonstrated that RNA induces the formation of tau aggregates in vitro and exhibits greater potency than other polyanions (Kampers T, et al. FEBS Letters. Dec. 16, 1996 1996; 399 (3): 344-349). The composition of tau-poly(A) RNA complexes were examined chromatographically and it was observed that the majority of tau assembles with RNA into chromatography stable complexes in vitro under standard physiologically relevant ionic and pH conditions. The assembled tau-poly(A) RNA complexes formed overnight and were resolved by native size exclusion chromatography followed by denaturing capillary electrophoresis yielding a range in approximate molecular weight from 330 kDa to 1250 kDa (FIG. 1D). This size range is consistent with the tau/RNA complexes being composed of medium N tau oligomers consisting of ˜3 to 24 tau monomers bound to poly(A) RNA. Since RNA is by far the most abundant tau binding macromolecule within the cytoplasm, the observation that tau/RNA interaction promotes oligomerization of tau supports a role for RNA in the initial phases of pathological tau aggregation in vivo. To further explore the relevance of poly(A) RNA induced tau oligomerization in animal models of tauopathy, transgenic C. elegans was generated that stably express a poly(A)₄₅ RNA transcript with the absence of any attached coding sequence in the cells. These poly(A) RNA expressing transgenic animals are viable, normal, and healthy. When crossed to pan-neuronal tau transgenic C. elegans the poly(A) RNA transgene significantly exacerbates neuronal dysfunction indicated by behavioral deficits (FIG. 1E). The poly(A)₄₅ transgene also exacerbated accumulation of pathological tau species (FIGS. 1F, 1G).

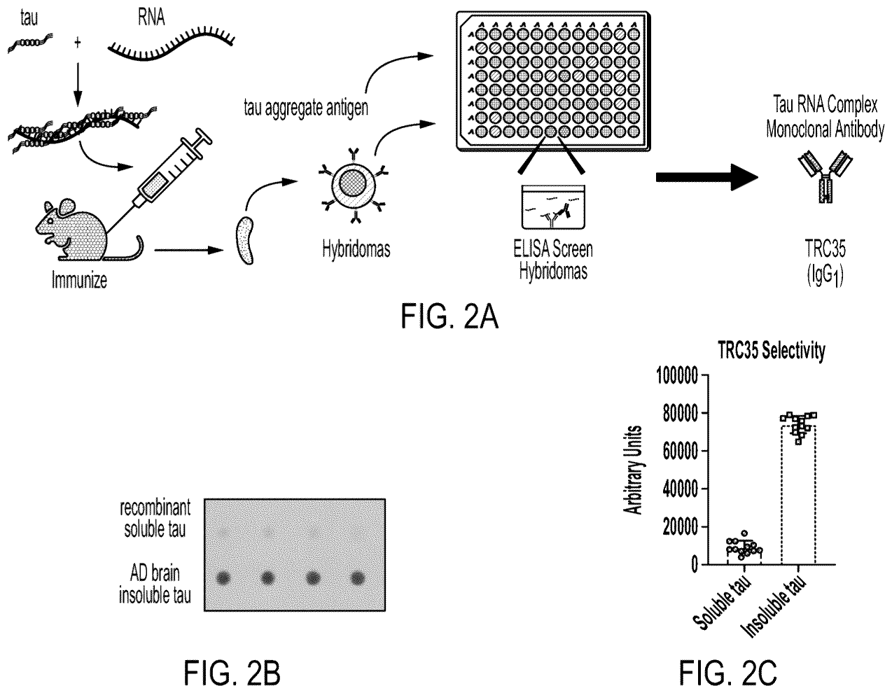

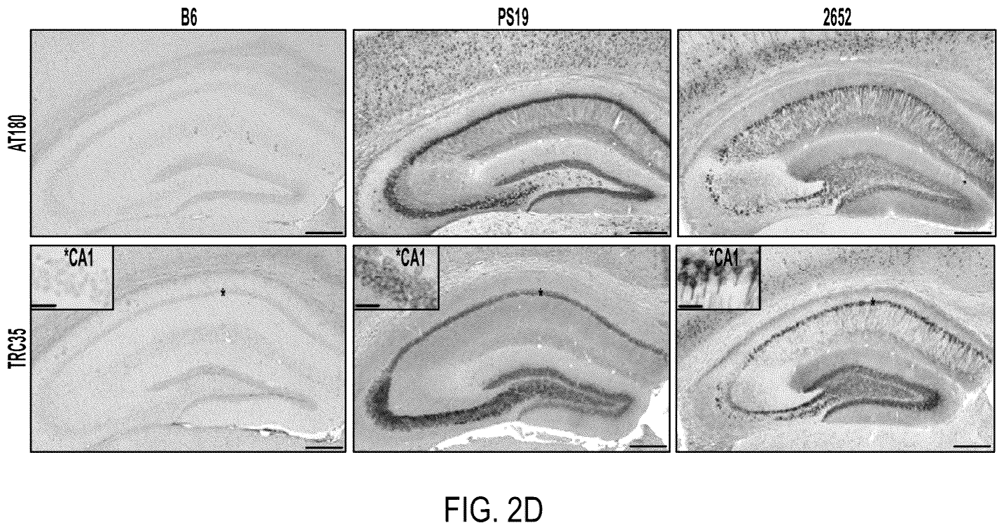

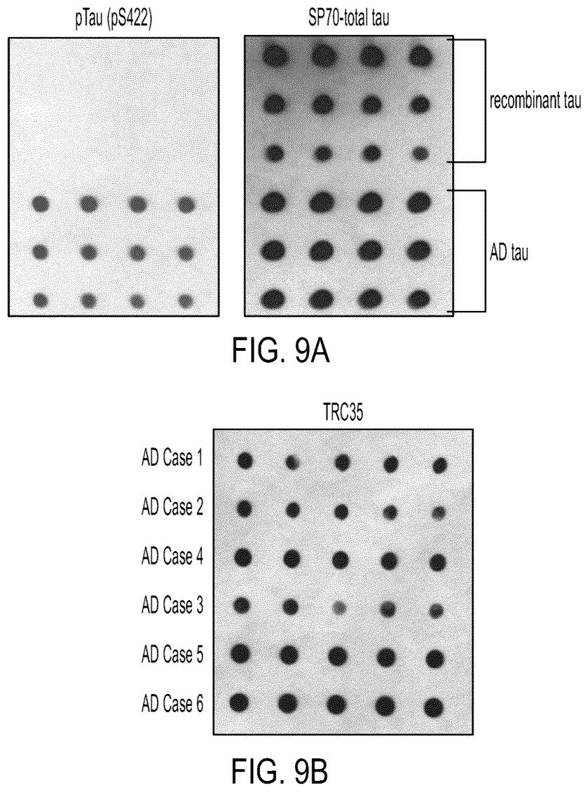

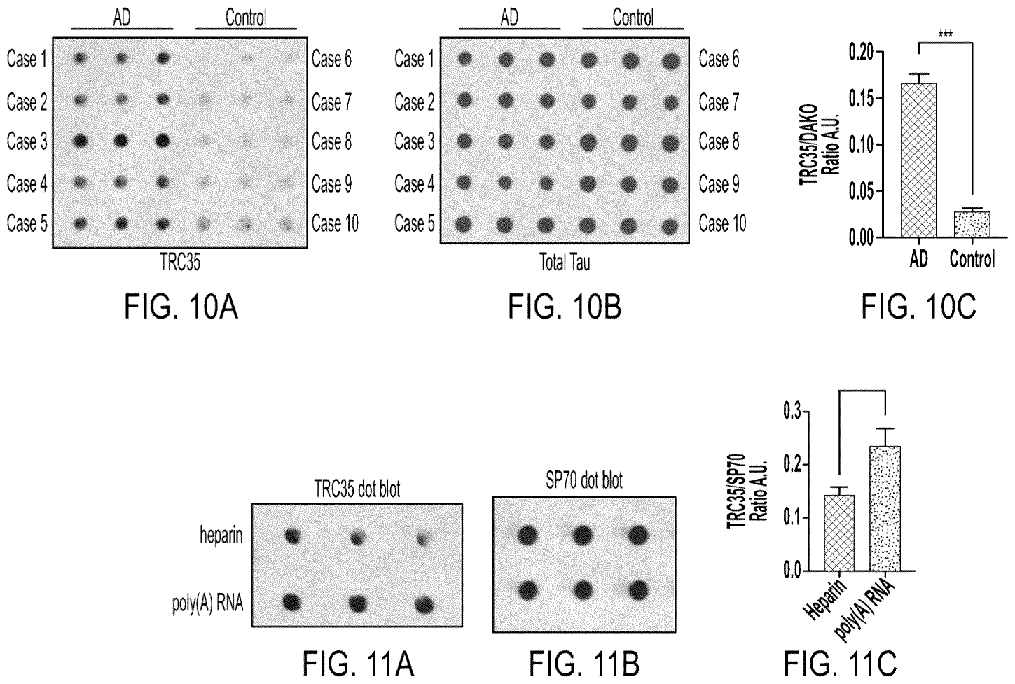

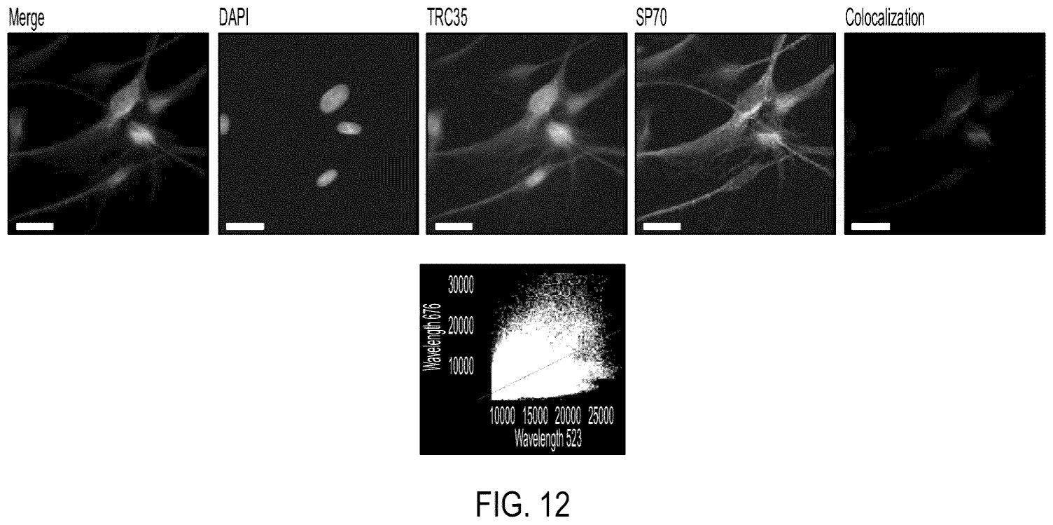

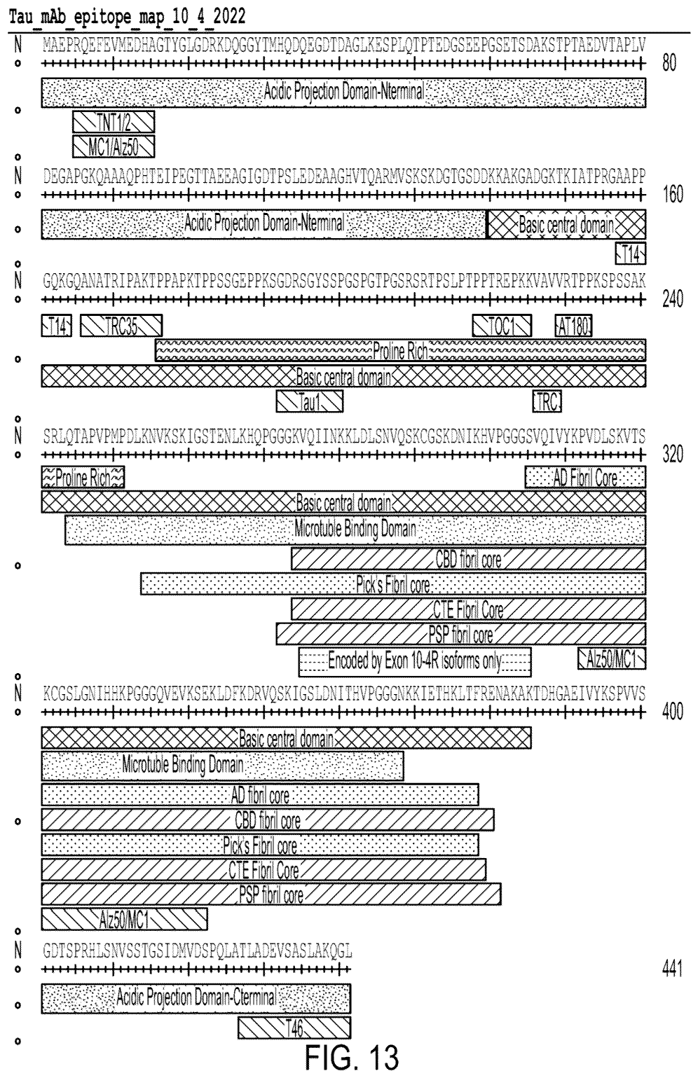

Mouse immunization with tau/RNA complexes to produce a monoclonal antibody recognizing a disease-specific conformational epitope of tau. To create a specific reagent for detecting tau/RNA complexes (TRCs) in disease states and model systems, in vitro assembled tau/RNA complexes were used as the immunogen to produce mouse monoclonal antibodies. Hybridoma cell lines were screened and a single hybridoma clone, #35, expressing a monoclonal antibody of the IgG₁ isotype was identified with selectivity for pathological tau assemblies and named TRC35 (FIG. 2A). Given the aggregated state of TRC antigen, the TRC35 mAb is expected to prefer tau assemblies over monomeric tau; a native protein dot immunoblot analysis was used to validate the specificity of TRC35. Recombinant tau lacks pathological tau species such as pTau, but loading controls with a total tau antibody reveal similar levels of total tau (FIG. 9). The data demonstrate that TRC35 recognizes detergent insoluble pathological tau purified from Alzheimer's disease brain tissue, but not monomeric recombinant human tau, with ˜8-fold selectivity (FIGS. 2B, 2C). Further, TRC35 reacts strongly with Alzheimer's disease brain lysate but not age matched control brain lysate (FIGS. 10A, 10B). In addition, the TRC35 reactive conformation tau is most strongly induced by incubation with poly(A) RNA, but heparin, a polyanion with similar charge density can also promote tau to adopt a TRC35 reactive conformation (FIGS. 11A, 11B). In addition, the accumulation of TRC35+ tau was examined in cultured human ReNcell VM neural progenitors differentiated into neurons (Choi S H, et al. Nature. Nov. 13 2014; 515 (7526): 274-8). ReNcell neurons exhibit TRC35+ accumulation throughout both the soma and nucleus, while SP70 tau monoclonal antibody detecting monomeric soluble tau is confined to the soma. Thus, TRC35 and soluble tau appear to partially overlap in cultured human neuronal cells (colocalization coefficient=0.6, FIG. 12). To map the specific tau epitope recognized by TRC35, a peptide mapping analysis was conducted and a discontinuous region just outside the proline rich domain was identified consisting of amino acids 166-176 and 225-228 (see FIG. 16, FIG. 13 peptide epitope sequence ANATRIPAKTP (SEQ ID NO: 122) and KVAV (SEQ ID NO: 124). Note that the epitope mapping of TRC35 revealed a discontinuous epitope consistent with a conformation dependent antibody. TRC35/tau binding did not require RNA or other polyanions demonstrating the TRC35 epitope appears to be a purely conformational peptide epitope. The c-terminal KVAV peptide component of the epitope appears immediately adjacent to the TOC1 epitope, but distinct (Ward S M, et al. J Alzheimers Dis. 2013; 37 (3): 593-602; and Ward S M, et al. Biochem Soc Trans. Aug. 1 2012; 40 (4): 667-71) and FIG. 16). Taken together these observations show tau binding to poly(A) RNA or perhaps other polyanions exposes the TRC35 epitope on tau when promoting tau aggregation, but RNA does not itself constitute part of the TRC35 epitope.

TRC35 recognizes a pre-tangle tau epitope in mouse models of tauopathy. To ascertain whether the TRC35 epitope becomes exposed on authentic neuronal fibrillary tau deposits, it was investigated whether two distinct transgenic mouse models of tauopathy, one exhibiting frank NFT accumulation and another that does not exhibit fibrillar tau. The perfused brains for P301S mutant human tau transgenic PS19 mice, a well-characterized mouse tauopathy model exhibiting progressive accumulation of Gallyas silver positive NFTs (Yoshiyama Y, et al. Neuron. Feb. 1 2007; 53 (3): 337-51) was examined. A transgenic mouse line driving high level pan-neuronal wild type human tau, Tau4RTg2652, which lacks any fibrillar tau and are Gallyas silver negative even at advanced age (Wheeler J M, et al. Acta neuropathological communications. 2015; 3:33) was also used. Immunostaining for phosphorylated tau (pTau Thr231—AT180) in the mouse hippocampus reveals similar levels of pTau in both tau transgenic strains (FIG. 2D). Immunostaining with TRC35 showed that this tau conformation is not identified in wild type mice but is present in both PS19 and Tau4RTg2652 animals. These findings demonstrate that TRC35 labels an epitope exposed by the accumulation of tau conformations occurring relatively early in the tauopathy cascade as it occurs in Tau4RTg2652 animals that do not exhibit frank NFT deposition, but do exhibit prominent pTau and pretangle tau accumulations.

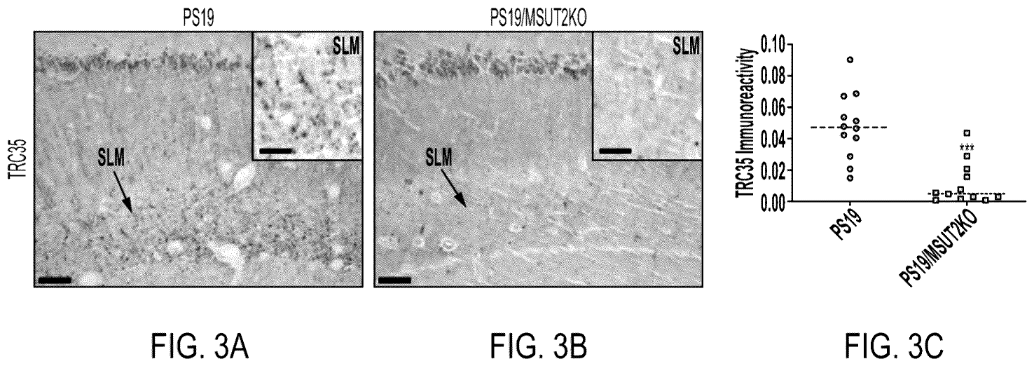

Perturbing poly(A) regulation decreases accumulation of TRC35 immunoreactivity. Sut-2 has been identified as a suppressor of tau-induced neurodegenerative defects in C. elegans (Guthrie C R, et al. Hum Mol Genet. May 15 2009; 18 (10): 1825-38). The sut-2 gene encodes a zinc finger protein with a single conserved homolog in diverse species ranging from yeast to humans and is thought to regulate poly(A) RNA tail lengths on mRNAs (Kelly S M, et al. RNA. May 2014; 20 (5): 681-8; and Baker J D, et al. PLOS Biol. June 2017; 15 (6): e2001336; Rha J, et al. Hum Mol Genet. Jun. 29 2017; and Soucek S, et al. Biochim Biophys Acta. June 2012; 1819 (6): 546-54). MSUT2 is the mammalian homolog of the C. elegans sut-2 gene; postmortem tissue studies suggest that human MSUT2 protein levels may influence neuronal vulnerability to tau toxicity and aggregation (Wheeler J M, et al. Sci Transl Med. Dec. 18 2019; 11 (523); and Guthrie C R, et al. Hum Mol Genet. May 15 2011; 20 (10): 1989-99). Further, MSUT2 gene knockout ameliorates tau neurodegeneration, tau pathology, and cognitive deficits in mouse models of tauopathy (Wheeler J M, et al. Sci Transl Med. Dec. 18 2019; 11 (523)). To test whether the TRC35+ pathological tau lesions are influenced by MSUT2 function, TRC35+ immunoreactivity in the stratum lacunosum moleculare (SLM) of PS19 was compared to PS19/MSUT2 KO mice. MSUT2 KO significantly decreases accumulation of TRC35 immunoreactivity in the hippocampus of PS19 mice by about 4-fold (FIG. 3). These findings replicate previous observations for hyperphosphorylated and fibrillar tau in PS19/MSUT2 KO mice (Wheeler J M, et al. Sci Transl Med. Dec. 18 2019; 11 (523)). Taken together, these findings show that mAb TRC35 labels pathological tau lesions in mice and that modulating poly(A) RNA availability influences tau/RNA complex formation in tauopathy mouse and worm models.

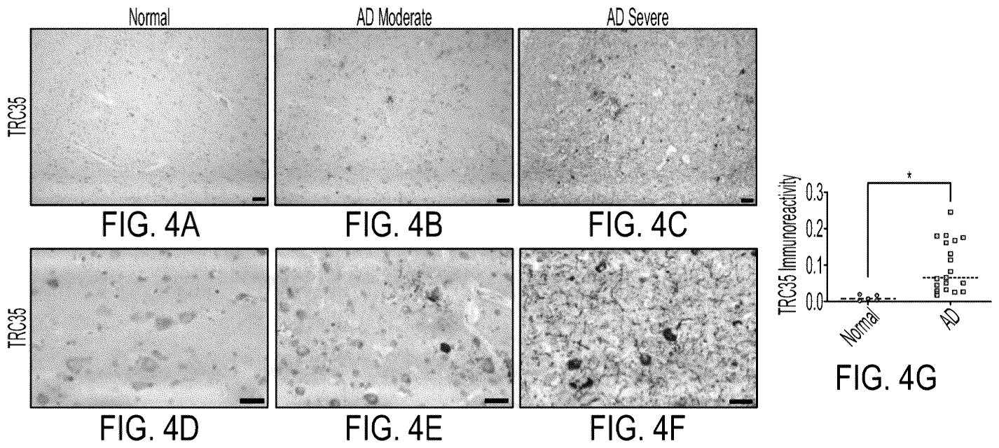

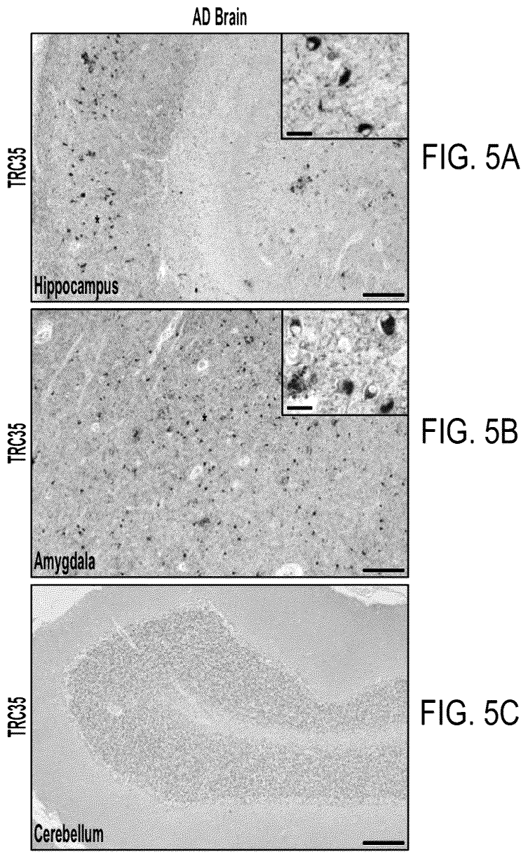

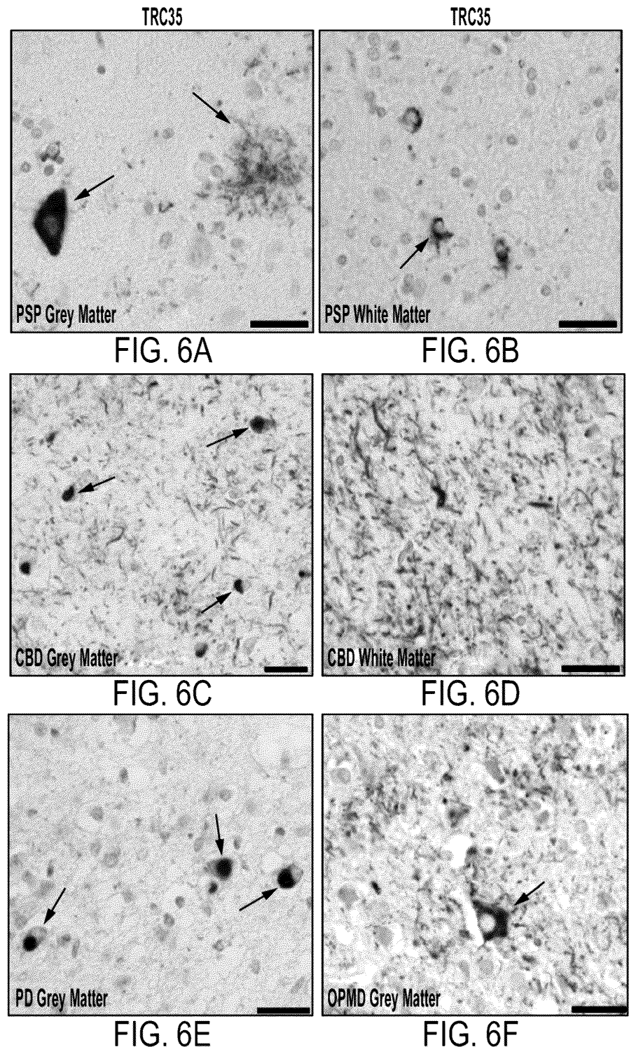

TRC35 labels abundant tau species in Alzheimer's disease but not controls. To further investigate the contribution of TRC35 immunoreactivity to the neurodegenerative cascade in tauopathy, brain sections from Alzheimer's disease and control brain tissues were immunostained with the TRC35 mAb. Immunoreactivity for TRC35 is low but detectable in normal controls with a diffuse neuronal soma staining pattern noticeably lacking detectable neuritic or fibrillary neuropathology (FIGS. 4A, 4D). In the frontal cortex from Alzheimer's disease brain donors, we observe varying degrees of TRC35 immunoreactivity (FIGS. 4B, 4C, 4E, 4F) was observed, with some cases exhibiting extensive fibrillar immunoreactivity in cortical neurons, dystrophic neurites, and neuropil threads (FIGS. 4C, 4F), while others demonstrated more modest cytoplasmic staining with regions of neuritic pathology (FIGS. 4B, 4E). The abundance of detectable TRC35 immunoreactivity is significantly higher in Alzheimer's disease as compared to cognitively normal age matched controls (FIG. 4G). The distribution of TRC35 containing lesions extend throughout the brain of Alzheimer's disease donors in regions with expected high levels of pathological tau, including the hippocampus and amygdala (FIGS. 5A, 5B). However, unlike in the frontal cortex, lesions in the hippocampus and amygdala were uniformly abundant and robustly TRC35+ across Alzheimer's disease cases. These brain regions are known to accumulate abundant tau pathology at an earlier stage in the disease process, showing TRC35 positive lesions may spread to the frontal cortex at later stages of the disease. TRC35 lesions were not observed in the cerebellum, a region typically spared from tau pathology in Alzheimer's disease (FIG. 5C). Other tauopathy disorders (FIG. 6) were also examined. Brain tissue from progressive supranuclear palsy donors exhibited both NFT like lesions and tufted astrocytes in grey matter (FIG. 6A) and TRC35+ oligodendroglial coils in subcortical white matter (FIG. 6B). Tissues from corticobasal degeneration donors exhibited staining in both TRC35+ neuropil threads and neuronal soma in grey matter and abundant dense TRC35+ neuropil threads in white matter (FIGS. 6C, 6D). Tissues from donors with Pick's disease exhibited globe like TRC35+ Pick bodies (FIG. 6E). Taken together, the pattern of neurons exhibiting TRC35 positivity parallels the pattern of pathological pre-tangle and pTau deposition in Alzheimer's disease and related tauopathy disorders.

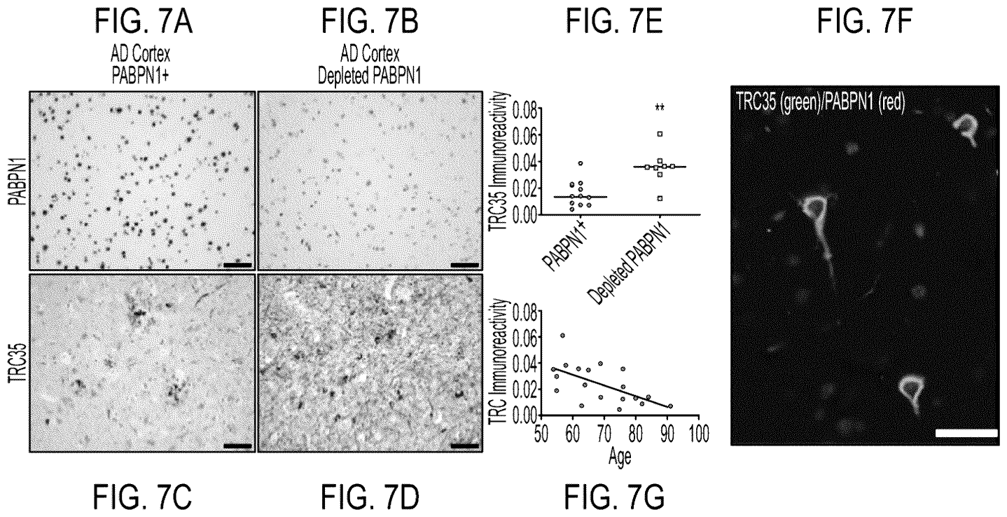

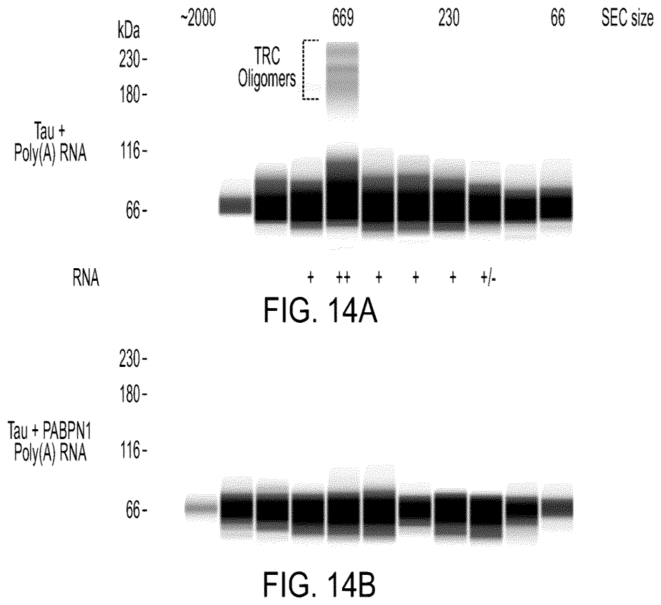

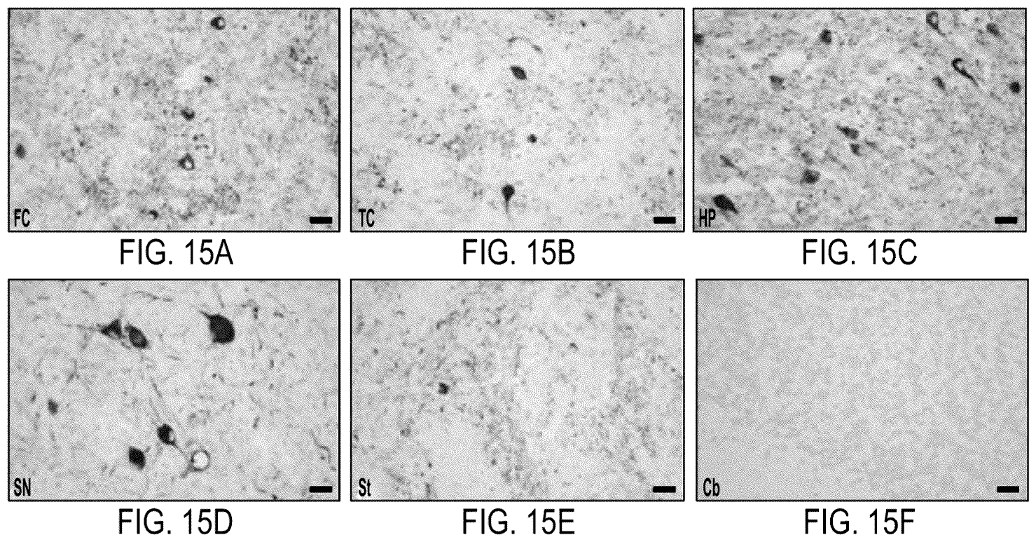

TRC35 was raised against the tau poly(A)/RNA complex. The nuclear poly(A) binding protein PABPN1 functions to protect and extend the length of the poly(A) tail on mRNAs (Kelly S M, et al. RNA. May 2014; 20 (5): 681-8; and Leung S W, et al. Gene. Jun. 15 2009; 439 (1-2): 71-8). It has been shown that PABPN1 also functions to protect against tauopathy in human cells where PABPN1 knockdown exacerbates tau accumulation (McMillan P J, et al. Acta neuropathologica communications. Jun. 29 2021; 9 (1): 117; and Wheeler J M, et al. Sci Transl Med. Dec. 18 2019; 11 (523)). PABPN1 also exhibits a reciprocal pattern of control over tau pathology relative to MSUT2. To assess whether PABPN1 impacts RNA mediated tau oligomerization, size exclusion chromatography was conducted on TRCs made from recombinant tau and poly(A) RNA. The presence of PABPN1 disrupted oligomeric high molecular weight tau species under size exclusion fractionation (FIG. 14). In human disease, PABPN1 loss of function occurs in oculopharyngeal muscular dystrophy (OPMD) caused by a repeat expansion in the first coding exon of the PABPN1 gene. Neuropathological investigation of rare brain autopsy cases of OPMD revealed clear evidence of tau neuropathology in striatum, substantia nigra, hippocampus, temporal cortex, and frontal cortex, while cerebellum was negative (FIG. 15); OPMD cases also exhibited prominent TRC35+ tau inclusions in these same regions (as demonstrated in frontal cortex, FIG. 6F). To further assess the interaction between TRC35 and PABPN1, the frontal cortex of a collection of Alzheimer's disease cases was immunostained for PABPN1. Alzheimer's disease cases with PABPN1 depletion in the frontal cortex (n=8, FIG. 7B) exhibited more severe accumulation of pathological tau as measured by TRC35 immunostaining, which included abundant apparent NFTs and dystrophic neurite profiles (FIGS. 7D, 7E). In contrast, cases with normal cortical PABPN1 staining (n=11, FIG. 7A) exhibited more modest TRC35 immunoreactivity characterized by sparse neuritic immunoreactivity and sporadic NFTs (FIG. 7C, 7E). Further, dual label immunofluorescence staining for PABPN1 and TRC35 showed that tangle bearing, TRC35+ neurons appear to exhibit diminished nuclear speckle PABPN1, although this could also be consistent with neurodegenerative changes (FIG. 7F). To explore whether TRC35 immunoreactivity varied in Alzheimer's disease with age at onset, TRC35+ immunostaining and age of disease onset was examined in postmortem brain tissue from the collection of Alzheimer's disease cases; the intensity of TRC35 immunoreactivity in the frontal cortex correlated with the age at disease onset in Alzheimer's disease (FIG. 7G, Pearson correlation coefficient=−0.59, p=0.007). Taken together, these findings demonstrate a relationship between TRC35 immunoreactivity and disease severity.

The genesis of pathological tau in normal aging, AD, and other tauopathy disorders remains incompletely understood. Pathological assembly of tau represents a gain of toxic function that disrupts cellular activities, contributing to neuronal demise in many aging related neurodegenerative disorders. Thorough study in Alzheimer's disease has demonstrated a clear connection between accumulation of tau containing pathological lesions, cognitive decline, and neuronal loss (Gomez-Isla T, et al. Ann Neurol. January 1997; 41 (1): 17-24; and Nelson P T, et al. J Neuropathol Exp Neurol. May 2012; 71 (5): 362-81). Early work in the field of tau biology identified RNA binding as a potential modulator of tau microtubule polymerization activity (Bryan J B, et al. Proc Natl Acad Sci USA. September 1975; 72 (9): 3570-4; and Schroder H C, et al. Mech Ageing Dev. January 1984; 24 (1): 101-17). Subsequently, a variety of distinct RNA binding proteins have been implicated as modifiers of tau accumulation and neurotoxicity (Lester E, et al. Neuron. Apr. 7 2021; Kow R L, et al. Neurobiol Dis. January 2021; 147:105148; Wheeler J M, et al. Sci Transl Med. Dec. 18 2019; 11 (523); Vanderweyde T, et al. Cell reports. May 17 2016; 15 (7): 1455-66; Apicco D J, et al. Nat Neurosci. January 2018; 21 (1): 72-80; Ash P E A, et al. Proc Natl Acad Sci USA. Mar. 2 2021; 118 (9); Montalbano M, et al. Nat Commun. Aug. 27 2020; 11 (1): 4305; Tanaka H, et al. Mol Psychiatry. October 2018; 23 (10): 2090-2110; Kosik K S and Han S. Adv Exp Med Biol. 2019; 1184:327-339; and Taylor L M, et al. FEBS J. July 2019; 286 (13): 2434-2446). Among these RNA binding proteins, MSUT2, PABPN1, TIA, PAP, and ALYREF directly interact with either poly(A) RNA or known poly(A) RNA binding proteins, and MSUT2 and PABPN1 can influence tauopathy related phenotypes and poly(A) tail lengths on mRNAs (Wheeler J M, et al. Sci Transl Med. Dec. 18 2019; 11 (523); Rha J, et al. Hum Mol Genet. Jun. 29 2017; and Leung S W, et al. Gene. Jun. 15 2009; 439 (1-2): 71-8). Taken together these previous observations led to testing whether tau binding to RNA leads to pathological consequences because tau/RNA binding precludes tau MT dimer binding impacting MT assembly (FIG. 1). Furthermore, co-expression of tau and poly(A)₄₅ in C. elegans neurons exacerbates tauopathy related behavioral phenotypes (FIG. 1E) and also exacerbates accumulation of pathological tau species including total and phosphorylated tau (FIGS. 1F, 1G). It was tested whether the molecular mechanism of this tauopathy exacerbation occurs because poly(A) RNA abundance exceeds poly(A) RNABP capacity, thus, exposing tau to naked RNA driving tau/RNA binding, subsequent tau oligomerization, and impaired tau proteostasis.

The data described herein demonstrate that tau binds RNA with high affinity, but low specificity, although it exhibits some preference for unstructured RNA over structured tRNA. Tau binds RNA with higher affinity than tubulin dimers, and RNA binding precludes tubulin binding to tau. Thus, RNA competes with tubulin for tau. The results disclosed herein recapitulated observations that poly(A) RNA inhibits tau activity in promoting microtubule polymerization, consistent with the binding studies. Further, when bound to poly(A) RNA, tau readily assembles into medium-N oligomers demonstrating that the formation of tau/RNA complexes may be on pathway to pathological aggregation. Co-expression of poly(A) RNA and tau drives stronger tauopathy related phenotypes including neuronal dysfunction and pathological tau accumulation (FIG. 1). To develop affinity reagents for probing the potential disease relevance of TRCs, TRCs were produced in vitro and employed as an immunogen. From TRC-immunized mice, we isolated hybridoma lines expressing TRC mAbs were isolated and exhibited strong preference for aggregated human AD-derived pathological tau over recombinant soluble tau (FIG. 2).

The TRC35 mAb was characterized by immunohistology and it specifically stains tau lesions in transgenic mouse brains from both the Tau4RTg2652 and PS19 models of tauopathy, but not non-transgenic mice. The neuropathological characterization of TRC35 immunoreactivity in PS19 and Tau4RTg2652 animals demonstrates that tau/RNA complexes occur prior to tau fibrillization in neurons. The consequence of MSUT2 knockout was also examined in the PS19 tauopathy model, resulting in dramatic decreases in other pathological tau species and it was observed that MSUT2 KO mice exhibited reduced accumulation of TRC35 immunoreactivity in the hippocampus.

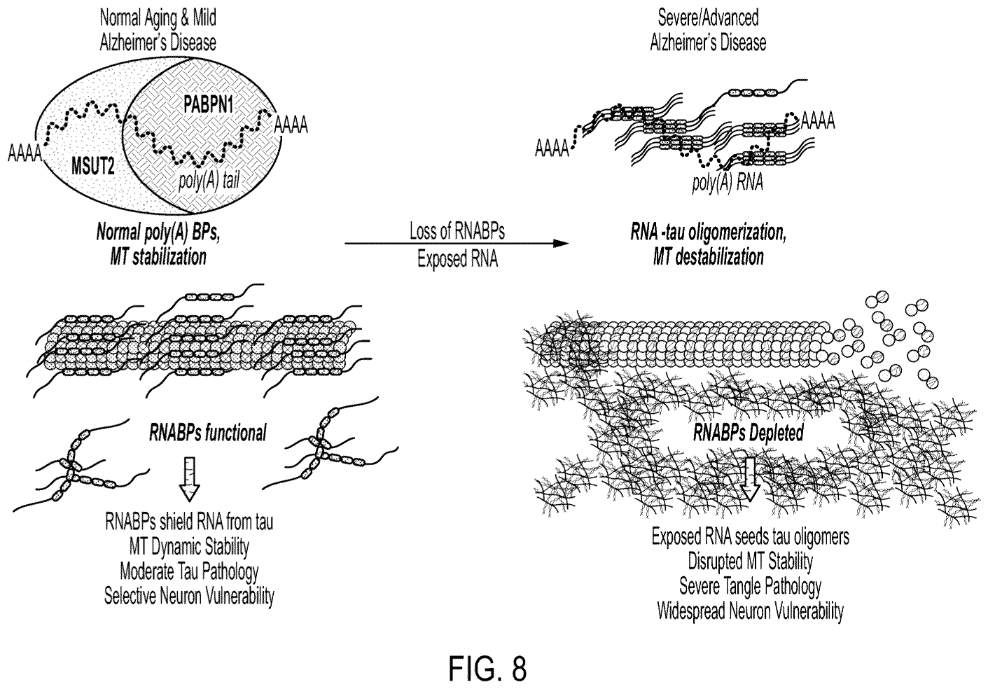

To explore the relevance of the accumulation of TRCs in disease, postmortem brain tissue from tauopathy donors was examined. In AD, it was observed that TRC35 positive somatodendritic staining, dystrophic neurites, neuropil threads, and frank tangles consistent with TRC depositing with pathological tau. Characterization of Alzheimer's disease and other tauopathy cases revealed that TRC35 labels pathological tau deposits in both 4R tauopathies (PSP and CBD) and 3R tauopathies (Pick's disease). Further, both 3R and 4R tau show high affinity for poly(A) RNA (FIG. 1). Examination of OPMD, a neuromuscular disease with onset of dementia caused by mutations in PABPN1, revealed evidence for tauopathy including pTau and TRC35 positive lesions. To explore the relationship between TRC35 and RNA binding proteins, the cohort of Alzheimer's disease cases were characterized with respect to PABPN1. A subset of Alzheimer's disease cases exhibited depletion of PABPN1 from the nucleus and robust accumulation of TRC35 lesions. The results show that tau binds RNA (Bryan J B, et al. Proc Natl Acad Sci USA. September 1975; 72 (9): 3570-4) and it may have greater access to RNA in these Alzheimer's disease cases in part because of reduced PABPN1 abundance competing for poly(A) RNA binding (FIG. 8). This finding can also be extended to other disorders including Alzheimer's disease and related disorders where depletion of RNA binding proteins occurs and may expose cytoplasmic RNAs to tau thereby promoting tauopathy through increased RNA mediated tau oligomerization. The findings disclosed herein support that TRC35 levels correlate with the age of Alzheimer's disease onset (FIG. 7G). Taken together, these results also support that tau protein access to poly(A) RNA may be constrained by RNA binding proteins.

Emerging evidence has shown that changes in RNA metabolism/processing through the actions of RNA binding proteins modulate pathological tau accumulation. Modeling tauopathy in C. elegans has uncovered genes encoding RNA binding proteins as translationally relevant modifiers of tauopathy, including sut-1, sut-2/MSUT2, parn-2/TOE1, aly-1, aly-2, and aly-3 (Kow R L, et al. Neurobiol Dis. January 2021; 147:105148; Wheeler J M, et al. Sci Transl Med. Dec. 18 2019; 11 (523); Guthrie C R, et al. Hum Mol Genet. May 15 2011; 20 (10): 1989-99; Guthrie C R, et al. Hum Mol Genet. May 15 2009; 18 (10): 1825-38; Kraemer B C and Schellenberg G D. Hum Mol Genet. 2007 Aug. 15 2007; 16 (16): 1959-71; and MacMorris M, et al. April 2007; 13 (4): 511-20). Parallel work has identified other poly(A) RNA binding proteins like T-cell intracellular antigen 1 (TIA1) which colocalizes with phase separated tau in stress granules and promotes fibrillary deposits of pathological tau (Ash P E A, et al. Proc Natl Acad Sci USA. Mar. 2 2021; 118 (9); Jiang L, et al. Acta Neuropathol. February 2019; 137 (2): 259-277; and Maziuk B F, et al. Acta neuropathologica communications. Aug. 1 2018; 6 (1): 71). Independently, the RNA binding protein Musashi (MSI) was found to associate with pathological tau oligomers and drive nuclear dysfunction (Montalbano M, et al. Nat Commun. Aug. 27 2020; 11 (1): 4305; and Sengupta U, et al. Acta neuropathologica communications. Oct. 26 2018; 6 (1): 1139. Multiple studies have demonstrated that tau neuropathology drives neurodegeneration by causing dysfunction of nuclear RNA processing events (reviewed in (Dicz L, and Wegmann S. Frontiers in neurology. 2020; 11:1056)). For instance, spliceosome abnormalities have been thought to cause cryptic RNA splicing leading to neurodegeneration in Alzheimer's disease and related disorders (Hsich Y C, et al. Cell reports. Oct. 8 2019; 29 (2): 301-316 c10). In addition, pathological tau can impair multiple nuclear functions including recruiting the nuclear speckle resident splicing protein SRRM2 into cytoplasmic aggregates (Lester E, et al. Neuron. Apr. 7 2021; and McMillan P J, et al. Acta neuropathologica communications. Jun. 29 2021; 9 (1): 117) and impairing nuclear pore complex function via co-aggregation of tau with the disordered region of Nup98 (Eftekharzadch B, et al. Neuron. Sep. 5 2018; 99 (5): 925-940 c7). Other defects have been found with the U1-70K which disrupts U1 snRNP function in splicing (Hales C M, et al. Brain Pathol. July 2014; 24 (4): 344-51; Bishof I, et al. J Biol Chem. Jul. 13 2018; 293 (28): 11047-11066; and Hales C M, et al. Proteomics. December 2016; 16 (23): 3042-3053). Another alternative splicing factor, TDP-43 has been shown to synergize with pathological tau in the context of AD, perhaps through an RNA binding mechanism (Latimer C S and Liachko N F. Geroscience. August 2021; 43 (4): 1627-1634; Latimer C S, et al. Acta neuropathologica communications. Jun. 7 2019; 7 (1): 91; and Tome S O, et al. Acta Neuropathol. May 2021; 141 (5): 795-799).

Previous work has implicated poly(A) RNA both as a strong influencer of tau MT stabilizing activity (Bryan J B, et al. Proc Natl Acad Sci USA. September 1975; 72 (9): 3570-4) and potent driver of tau aggregation (Kampers T, et al. FEBS Lett. Dec. 16 1996; 399 (3): 344-9). Consistent with this, the results described herein show that tau binds poly(A) RNA with much greater affinity than tubulin dimers (FIG. 1C) or polymerized microtubules (MTs) (Butner K A and Kirschner M W. J Cell Biol. November 1991; 115 (3): 717-30; and Kadavath H, et al. Proc Natl Acad Sci USA. Jun. 16 2015; 112 (24): 7501-6) and disrupts tau-dependent MT assembly (FIG. 1A). In Hela cells, cytoplasmic RNA appears approximately 3-fold more abundant than tubulin (˜27 pg RNA in HeLa cell cytoplasm (Piwnicka M, et al. Cytometry. January 1983; 3 (4): 269-75) vs ˜7 pg tubulin (Finka A and Goloubinoff P. Cell Stress Chaperones. September 2013; 18 (5): 591-605; Bulinski J C and Borisy G G. Proc Natl Acad Sci USA. January 1979; 76 (1): 293-7)). The relative abundance of tubulin vs RNA remains unknown in human brain neurons, but tubulin/RNA competition for tau binding should be considered as a possible factor relevant to tau aggregation. Thus, tau may bind RNA preferentially when other RNA binding proteins fail to shield cytoplasmic RNA from access to tau. Clearly many other RNA binding proteins play a role in modulating tauopathy (Lester E, et al. Neuron. Apr. 7 2021; McMillan P J, et al. Acta neuropathologica communications. Jun. 29 2021; 9 (1): 117; Ash P E A, et al. Proc Natl Acad Sci USA. Mar. 2 2021; 118 (9); Montalbano M, et al. Nat Commun. Aug. 27 2020; 11 (1): 4305; Jiang L, et al. Acta Neuropathol. February 2019; 137 (2): 259-277; Maziuk B F, et al. Acta neuropathologica communications. Aug. 1 2018; 6 (1): 71; Sengupta U, et al. Acta neuropathologica communications. Oct. 26 2018; 6 (1): 113; Hales C M, et al. Brain Pathol. July 2014; 24 (4): 344-51; Bishof I, et al. J Biol Chem. Jul. 13 2018; 293 (28): 11047-11066; Hales C M, et al. Proteomics. December 2016; 16 (23): 3042-3053; Latimer C S and Liachko N F. Geroscience. August 2021; 43 (4): 1627-1634; Latimer C S, et al. Acta neuropathologica communications. Jun. 7 2019; 7 (1): 91; and Tome S O, et al. Acta Neuropathol. May 2021; 141 (5): 795-799) which, in sum, supports mechanism relating tau/RNA binding with tau MT function and tau neuropathology. As described herein, it was demonstrated that PABPN1 loss of function in OPMD provokes pathological tau accumulation (FIG. 6). Further support for these findings comes from the observation that RNA binds to tau fibrils and is required to sustain multiple rounds of seeding competent tau using in vitro propagation of tau derived seeds to drive fibril formation (Dinkel P D, et al. Biochemistry. Aug. 4 2015; 54 (30): 4731-40).

Prior study of tau aggregation yielded a diverse group of monoclonal antibodies with high utility that define conformation specific epitopes including Alz50 (pre-tangle tau, Wolozin B L, et al. Science. May 2 1986; 232 (4750): 648-50), MC1 (pretangle tau, Jicha G A, et al. J Neurosci Res. Apr. 15 1997; 48 (2): 128-32; and Weaver C L, et al. Neurobiol Aging. September-October 2000; 21 (5): 719-27), TOC1 (Oligomer/Dimer, Ward S M, et al. J Alzheimers Dis. 2013; 37 (3): 593-602), TOMA (oligomer, Castillo-Carranza D L, et al. J Alzheimers Dis. 2014; 40 Suppl 1: S97-S111), GT38 (fibrillar tau, Gibbons G S, et al. J Neuropathol Exp Neurol. Mar. 1 2018; 77 (3): 216-228). As described herein, TRC35 is a conformation dependent selective tau monoclonal antibody that can be used for detecting pathological tau oligomers seeded by RNA or perhaps other polyanions. For TRC35, the complex epitope consists of two discontinuous peptide motifs flanking the tau proline rich domain, which is predicted to be an unstructured region in tau fibril cores for tauopathies solved to date (reviewed in 8). Pathological RNA access to cytoplasmic tau through RNA binding protein deficiency or ribostatic derangement drives the accumulation of tau oligomers exposing the TRC35 epitope on tau-positive lesions in Alzheimer's disease and related dementia disorders. However, it remains possible that other polyanions could promote TRC35 reactivity in specific circumstances (for instance with extracellular tau).

Disclosed herein is data that provides evidence supporting relevance of TRCs in disease. The finding that RNA seeded tau aggregates occur in disease provided evidence for focusing on the RNA binding activity of tau in pathology, and can be used as a therapeutic to target pathological tau for Alzheimer's disease and related disorders.

TABLE 2
Sequences of antibody TRC35 and antibody TRC1.

| Name | SEQUENCE |
|---|---|
| TRC35 | QVQLQQPGTELVKPGASVKLSCKASGYTFTSYWLQWVKQRPGQGL |
| Heavy Chain | EWIGEIDPSDSYTNYNQKFKGKATLTVDTSSSTAYMQLSSLTSEDSA |
| Variable | VYYCATLTGSWGQGTTLAVSS (SEQ ID NO: 1) |
| Region | |
| TRC35 Light | DVLLTQTPLSLPVSLGDQASISCRASQSIVDSNGNTFLEWYLQKSGQ |
| (Kappa) | SPKVLIYKVSNRFSGVPDRFSGRGSGTDFTLKISRVEAEDLGVYYCF |
| Chain | QGSHVPYTFGGGTKLEIK (SEQ ID NO: 2) |
| Variable | |
| Region | |
| Recombinant | mQVQLQQPGTELVKPGASVKLSCKASGYTFTSYWLQWVKQRPGQ |
| TRC35_scFv | GLEWIGEIDPSDSYTNYNQKFKGKATLTVDTSSSTAYMQLSSLTSED |
| sequence | SAVYYCATLTGSWGQGTTLAVSSggggsggggsggggsggggsDVLLTQTPL |
| | SLPVSLGDQASISCRASQSIVDSNGNTFLEWYLQKSGQSPKVLIYKVS |
| | NRFSGVPDRFSGRGSGTDFTLKISRVEAEDLGVYYCFQGSHVPYTFG |
| | GGTKLEIKLPYTGHHHHHH (SEQ ID NO: 3) |
| Humanized | MDYKDHDGDYKDHDIDYKDDDDKQVQLvQsGaEvkKPGASVKvSC |
| TRC35 scFv | KASGYTFTSYWmhWVrQaPGQGLEWmGEIDPSDSYTsYaQKFqGrvT |
| | mTrDTStSTvYMeLSSLrSEDtAVYYCATLTGSWGQGTTLAVSSggggs |
| | ggggggggsDVvmTQsPLSLPVtLGqpASISCRASQSIVDSNGNTFLEWfq |
| | QrpGQSPrrLIYKVSNRdSGVPDRFSGsGSGTDFTLKISRVEAEDvGVY |
| | YCFQGSHVPYTFGGGTKLEIKYPYDVPDYAHHHHHH (SEQ ID NO: 4) |
| TRC35 | MDYKDHDGDYKDHDIDYKDDDDKQVQLQQPGTELVKPGASVKL |
| bioProtac | SCKASGYTFTSYWLQWVKQRPGQGLEWIGEIDPSDSYTNYNQKFK |
| | GKATLTVDTSSSTAYMQLSSLTSEDSAVYYCATLTGSWGQGTTLA |
| | VSSGGGGSGGGGSGGGGSDVLLTQTPLSLPVSLGDQASISCRASQSI |
| | VDSNGNTFLEWYLQKSGQSPKVLIYKVSNRFSGVPDRFSGRGSGTD |
| | FTLKISRVEAEDLGVYYCFQGSHVPYTFGGGTKLEIKYPYDVPDYA |
| | GGGGSGGGGSGGGGSGGGGSRLNFGDDIPSALRIAKKKRWNSIEER |
| | RIHQESELHSYLSRLIAAERERELEECQRNHEGDEDDSHVRAQQACIE |
| | AKHDKYMADMDELFSQVDEKRKKRDIPDYLCGKISFELMREPCITPS |
| | GITYDRKDIEEHLQRVGHFDPVTRSPLTQEQLIPNLAMKEVIDAFISEN |
| | GWVEDY (SEQ ID NO: 5) |
| TRC 35, | GYTFTSYW (SEQ ID NO: 6) |
| heavy chain, | |
| CDR1 | |
| TRC 35, | IDPSDSYT (SEQ ID NO: 7) |
| heavy chain, | |
| CDR2 | |
| TRC 35, | A (SEQ ID NO: 8) |
| heavy chain, | |
| CDR3 | |
| TRC35 light | QSIVDSNGNTF (SEQ ID NO: 9) |
| (Kappa) | |
| chain, CDR1 | |
| TRC35 light | KVS (SEQ ID NO: 10) |
| (Kappa) | |
| chain, CDR2 | |
| TRC35 light | FQGSHVP (SEQ ID NO: 11) |
| (Kappa) | |
| chain, CDR2 | |
| TRC1 heavy | QVQLQQPGAELVKPGASVKLSCKASDYTFTNYWIQWVKQRPGQGL |
| Chain | EWIGEIDPSDNYTNCNPEFKDKATLTVDTSSSTAYMHLNSLTSEDSA |
| Variable | VYYCGNTKGWWGQGTLVTVSA (SEQ ID NO: 12) |
| Region | |
| TRC1 light | DVLMTQTPLSLPVSLGDQASISCRSSQNIVNSNGDTYLEWYLQKPGQ |
| (Kappa) | SPKLLIYKVSNRFSGVPDRFSGSGSGTDFTLKISGAEAEDLGVYYCFQ |
| Chain | GSHVPLTFGAGTKLELK (SEQ ID NO: 13) |
| Variable | |
| Region | |
| TRC 1, | DYTFTNYW (SEQ ID NO: 14) |
| heavy chain, | |
| CDR1 | |
| TRC 1, | IDPSDNYT (SEQ ID NO: 15) |
| heavy chain, | |
| CDR2 | |
| TRC1 light | QNIVNSNGDTY (SEQ ID NO: 17) |
| (Kappa) | |
| chain, CDR1 | |
| TRC1 light | KVS (SEQ ID NO: 18) |
| (Kappa) | |
| chain, CDR2 | |
| TRC1 light | FQGSHVP (SEQ ID NO: 19) |
| (Kappa) | |
| chain, CDR3 | |
| Recombinant | mQVQLQQPGAELVKPGASVKLSCKASDYTFTNYWIQWVKQRPGQ |
| TRC1_scFv | GLEWIGEIDPSDNYTNCNPEFKDKATLTVDTSSSTAYMHLNSLTSED |
| sequence | SAVYYCGNTKGWWGQGTLVTVSAggggsggggsggggsDVLMTQTPLSL |
| | PVSLGDQASISCRSSQNIVNSNGDTYLEWYLQKPGQSPKLLIYKVSNR |
| | FSGVPDRFSGSGSGTDFTLKISGAEAEDLGVYYCFQGSHVPLTFGAGT |
| | KLELKGHHHHHH (SEQ ID NO: 16) |
| Sequence | MAEPRQEFEVMEDHAGTYGLGDRKDQGGYTMHQDQEGDTDAGLK |
| used for Tau- | ESPLQTPTEDGSEEPGSETSDAKSTPTAEDVTAPLVDEGAPGKQAAA |
| mAb epitope | QPHTEIPEGTTAEEAGIGDTPSLEDEAAGHVTQARMVSKSKDGTGSD |
| map | DKKAKGADGKTKIATPRGAAPPGQKGQANATRIPAKTPPAPKTPPSS |
| | GEPPKSGDRSGYSSPGSPGTPGSRSRTPSLPTPPTREPKKVAVVRTPPK |
| | SPSSAKSRLQTAPVPMPDLKNVKSKIGSTENLKHQPGGGKVQIINKKL |
| | DLSNVQSKCGSKDNIKHVPGGGSVQIVYKPVDLSKVTSKCGSLGNIH |
| | HKPGGGQVEVKSEKLDFKDRVQSKIGSLDNITHVPGGGNKKIETHKL |
| | TFRENAKAKTDHGAEIVYKSPVVSGDTSPRHLSNVSSTGSIDMVDSP |
| | QLATLADEVSASLAKQGL (SEQ ID NO: 20) |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An isolated antibody comprising a light chain variable region and a heavy chain variable region, wherein the light chain variable region comprises a complementarity determining region light chain 1 (CDRL1) amino acid sequence of SEQ ID NO: 9; a determining region light chain 2 (CDRL2) amino acid sequence of SEQ ID NO: 10; and a determining region light chain 3 (CDRL3) amino acid sequence of SEQ ID NO: 11; and wherein the heavy chain variable region comprises a complementarity determining region heavy chain 1 (CDRH1) amino acid sequence of SEQ ID NO: 6; a complementarity determining region heavy chain 2 (CDRH2) amino acid sequence of SEQ ID NO: 7; and a complementarity determining region heavy chain 3 (CDRH3) amino acid sequence of SEQ ID NO: 8.

2. The isolated antibody of claim 1, comprising a light chain variable region amino acid sequence of SEQ ID NO: 2.

3. The isolated antibody of claim 1, comprising a heavy chain variable region amino acid sequence of SEQ ID NO: 1.

4. (canceled)

5. The isolated antibody of claim 1, wherein the light chain variable region has an amino acid sequence that is at least 90% identical to the amino acid sequence of SEQ ID NO: 2.

6. The isolated antibody of claim 1, wherein the heavy chain variable region has an amino acid sequence that is at least 90% identical to the amino acid sequence of SEQ ID NO: 1.

7. An isolated antibody comprising a light chain variable region and a heavy chain variable region, wherein the light chain variable region comprises a complementarity determining region light chain 1 (CDRL1) amino acid sequence of SEQ ID NO: 17; a determining region light chain 2 (CDRL2) amino acid sequence of SEQ ID NO: 18; and a determining region light chain 3 (CDRL3) amino acid sequence of SEQ ID NO: 19; and wherein the heavy chain variable region comprises a complementarity determining region heavy chain 1 (CDRH1) amino acid sequence of SEQ ID NO: 14; and a complementarity determining region heavy chain 2 (CDRH2) amino acid sequence of SEQ ID NO: 15.

8. The isolated antibody of claim 7, comprising a light chain variable region amino acid sequence of SEQ ID NO: 13.

9. The isolated antibody of claim 7, comprising a heavy chain variable region amino acid sequence of SEQ ID NO: 12.

10. (canceled)

11. The isolated antibody of claim 7, wherein the light chain variable region has an amino acid sequence that is at least 90% identical to the amino acid sequence of SEQ ID NO: 13.

12. The isolated antibody of claim 7, wherein the heavy chain variable region has an amino acid sequence that is at least 90% identical to the amino acid sequence of SEQ ID NO: 12.

13. The isolated antibody of claim 1, wherein the antibody is recombinantly engineered, chimerized, or humanized.

14. The isolated antibody of claim 1, wherein the isolated antibody is a Fab, an Fab′, an F(ab′)₂, a Fv, a scFv, a diabody or fragments thereof.

15.-23. (canceled)

24. A method of treating a tauopathy in a subject, the method comprising administering to the subject a therapeutically effective amount of the isolated antibody of claim 1 or a fragment thereof.

25. A method of treating a tauopathy in a subject, the method comprising administering to the subject a therapeutically effective amount of the isolated antibody of claim 7 or a fragment thereof.

26. A method of treating dementia in a subject, the method comprising administering to the subject a therapeutically effective amount of the isolated antibody of claim 1 or a fragment thereof.

27. A method of treating dementia in a subject, the method comprising administering to the subject a therapeutically effective amount of the isolated antibody of claim 7 or a fragment thereof.

28. A method of treating inhibiting microtubule polymerization in a subject, the method comprising administering to the subject a therapeutically effective amount of the isolated antibody of claim 1 or a fragment thereof.

29. A method of inhibiting microtubule polymerization in a subject, the method comprising administering to the subject a therapeutically effective amount of the isolated antibody of claim 7 or a fragment thereof.

30. A method of treating ocular pharyngeal muscular dystrophy in a subject, the method comprising administering to the subject a therapeutically effective amount of the isolated antibody of claim 1 or a fragment thereof.

31. A method of treating ocular pharyngeal muscular dystrophy in a subject, the method comprising administering to the subject a therapeutically effective amount of the isolated antibody of claim 7 or a fragment thereof.

32.-37. (canceled)
